(19) United States
(12) Patent Application Publication
(10) Pub. No.: US 2025/0135006 A1
(43) Pub. Date: May 1, 2025

(54) AMPHIPHILIC TLR7/8 ADJUVANTS AND USES THEREOF

(71) Applicant: Pfizer Inc., New York, NY (US)

(72) Inventors: Derek William Bartlett, Chapel Hill, NC (US); Zoltan Beck, Rockville, MD (US); Andrew Fensome, Harvard, MA (US); Mark Edward Flanagan, Gales Ferry, CT (US); Michael William Pride, Staten Island, NY (US)

(21) Appl. No.: 18/763,016

(22) Filed: Jul. 3, 2024

Related U.S. Application Data
(60) Provisional application No. 63/660,632, filed on Jun. 17, 2024.
(60) Provisional application No. 63/512,402, filed on Jul. 7, 2023.

(51) Int. Cl. A61K 47/22; A61K 31/496; A61K 39/39; A61K 47/28

(52) U.S. Cl. CPC A61K 47/22; A61K 31/496; A61K 39/39; A61K 47/28

(57) ABSTRACT

The invention relates to compounds of Formula (I)

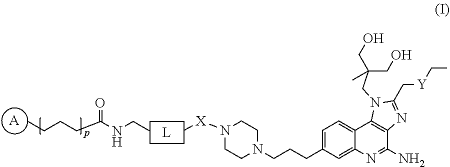

and pharmaceutically acceptable salts thereof, wherein A, L, X, Y and p are as defined in the description; to their use in medicine; to compositions containing them; to processes for their preparation; and to intermediates used in such processes. The compounds of Formula (I) may modulate the activity of antigens of interest and may be useful in inducing or enhancing an immune response against diseases, disorders and conditions mediated by antigens of interest.

### REFERENCE TO SEQUENCE LISTING

This application is being filed electronically via EFS-Web and includes an electronically submitted sequence listing in .xml format. The .xml file contains a sequence listing entitled “PC072985A Sequence Listing.xml” created on Jun. 17, 2024 and having a size of 6.02 KB. The sequence listing contained in this .xml file is part of the specification and is incorporated herein by reference in its entirety.

### BACKGROUND

The present invention relates to novel amphiphilic Toll-like receptor 7 (TLR7), Toll-like receptor 8 (TLR8), and Toll-like receptor 7/8 (TLR7/8) adjuvant compounds. The invention also relates to the preparation of the compounds and intermediates used in the preparation, compositions containing the compounds, and uses of the compounds, including as adjuvants for antigens of interest.

Studies and research regarding adjuvant use as a vaccine component have significantly increased nowadays. Adjuvants are compounds that enhance immune system activation and recognition of a vaccine's active component, especially concerning subunit-based vaccines.

Adjuvants act by enhancing the innate immune system's response magnitude, breadth, and durability. The potency of adjuvants is closely related to their ability to be recognized as pathogens/foreign bodies through pattern recognition receptors (PRRs). It is expected that a vaccine's active component will become recognized, thereby triggering a specific and long-lasting immune response to be mounted through the adaptive immune system (Excler et al., Nat. Med., 27 (2021), pp. 591-600).

Toll-like receptors (TLRs) are a family of transmembrane proteins that recognize structurally conserved molecules that are derived from and unique to pathogens, referred to as pathogen-associated molecular patterns (PAMPs), a sub-class of PRR. As such, TLRs function in the mammalian immune system as front-line sensors of pathogen-associated molecular patterns, detecting the presence of invading pathogens (Takeuchi and Akira 2010 Cell 140:805-820). TLR engagement in sentinel immune cells causes biosynthesis of selected cytokines (e.g., type I interferons), induction of costimulatory molecules, and increased antigen presentation capacity. These are important molecular mechanisms that activate innate and adaptive immune responses. Accordingly, agonists and antagonists of TLRs find use in modulating immune responses. TLR agonists are typically employed to stimulate immune responses, whereas TLR antagonists are typically employed to inhibit immune responses (Gosu etal 2012. Molecules 17:13503-13529).

The human genome contains 10 known functional TLRs, of these TLR3, TLR7, TLR8, and TLR9 sense nucleic acids and their degradation products. The distribution of TLR7, TLR8, and TLR9 is restricted to the endosomal compartments of cells and they are preferentially expressed in cells of the immune system. In the activated, dimeric receptor configuration TLR7 and TLR8 recognize single strand RNA at one ligand binding site and the ribonucleoside degradation products guanosine and uridine, respectively, (as well as small molecule ligands with related structural motifs) at a second ligand binding site (Zhang et al 2016 Immunity 45(4); 737-748: Tanji et al 2015 Nat Struct Mol Biol 22: 109-115). Engagement of TLR7 in plasmacytoid dendritic cells leads to the induction of Type I interferon, which plays essential functions in the control of the adaptive immune response (Bao and Liu 2013 Protein Cell 4:40-5). Engagement of TLR8 in myeloid dendritic cells, monocytes and monocyte-derived dendritic cells induces a prominent pro-inflammatory cytokine profile, characterized by increased production of tumor necrosis factor alpha, interleukin-12, and IL-18 (Eigenbrod et al J Immunol, 2015, 195, 1092-1099). Thus, virtually all major types of monocytic and dendritic cells can be activated by agonists of TLR7 and TLR8 to become highly effective antigen-presenting cells, thereby promoting an effective innate and adaptive immune response. Most antigen presenting cell types express only one of these two receptors, accordingly small molecules with potent agonist activity against both TLR7 and TLR8 receptors are potentially more effective immune adjuvants than agonists specific for only one of these TLRs. Thus, a TLR7/TLR8 (TLR7/8) small molecule agonist with dual bioactivity would cause innate immune responses in a wider range of antigen presenting cells and other key immune cell types, including plasmacytoid and myeloid dendritic cells, monocytes, and B cells (van Haren et al 2016 J Immunol 197:4413-4424; Ganapathi et al 2015 Plos One 10(8).e0134640).

Albumin has been extensively studied as a natural vector for lymph node targeted drug delivery (Adv. Drug Delivery Rev. 2018, 130, 73-89). First, albumin (about 7 nm in size) is the most abundant protein in the blood, reaching a concentration of 40 mg/mL, but maintaining a relatively lower concentration, about 14 mg/mL, in the interstitial fluid. This concentration difference tends to drive albumin to the lymphatics instead of blood capillaries. Second, albumin has an extraordinarily long half-life and is continuously synthesized in the liver for circulation. Third, natural ligands, such as long aliphatic fatty acids and hydrophobic molecules, have been discovered to bind albumin with their complexed crystal structures also being resolved. The albumin hitchhiking lymph node targeting was first demonstrated with TLR9 ligands conjugated to lipids and natural hydrophobic molecules such as cholesterol (Nature 507, 519-522, 2014). More recently this approach has been applied to a TLR7/8 agonist linked to cholesterol via a poly-dispersed PEG linker (Angew. Chem. Int. Ed. 2021, 60, 9467-9473).

Schotsaert and colleagues (Angew. Chem. Int. Ed. 2021, 60, 9467-9473, WO2022076723) have described a TLR7/8 adjuvant, exemplified by an imidazoquinazoline conjugated through a poly-dispersed PEG linker to an unnatural cholesterol amide as albumin binder.

What is needed in the field is a TLR7/8 agonist targeted to accumulate in the lymph node to (a) activate the immune cells in the lymph node to adjuvant the vaccine antigen, while (b) minimizing systemic exposure to the TLR7/8 agonist in order to circumvent systemic inflammation.

The current invention relates to the use of potent dual TLR7/8 agonists conjugated through a simple monomeric (mono-dispersed) polyethylene glycol (PEG) linker to a lipid. The mono-dispersed PEG linker system is advantageous over poly-dispersed PEGs. With a mono-dispersed PEG linker system the adjuvant is a single homogeneous molecular structure, while poly-dispersed PEG derived adjuvants consist of many molecules across a distribution of molecular weights with varying abundances. With the mono-dispersed PEG linker the single molecular structure provides advantages in terms of manufacturing a consistent product to administer to a subject, with a single set of physical properties with further advantages for formulation and delivery, pharmacokinetics and distribution, and pharmacology.

Accordingly, there remains a need for improved adjuvants targeted to accumulate in the lymph node to (a) activate the immune cells in the lymph node to adjuvant a vaccine antigen, while (b) minimizing systemic exposure to the TLR 7/8 agonist in order to_circumvent systemic inflammation.

### SUMMARY OF THE INVENTION

The present invention provides, in part, compounds of Formula (I) and pharmaceutically acceptable salts thereof. Such compounds may agonize or modulate the activity of TLR7 and/or TLR8 and may be useful as vaccine adjuvants. Also provided are pharmaceutical compositions, comprising the compounds or salts of the invention, alone or in combination with additional therapeutic agents. The present invention also provides, in part, methods for preparing such compounds, pharmaceutically acceptable salts and compositions of the invention, and methods of using the foregoing. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

According to an embodiment of the invention there is provided a compound of Formula (I)

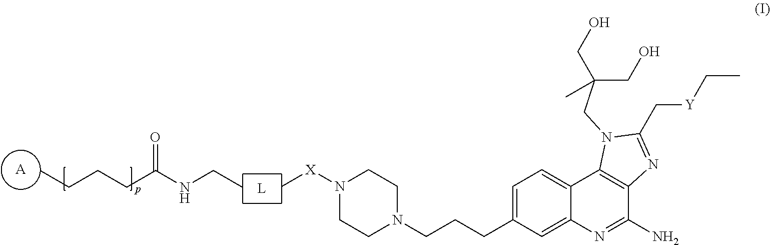

or a pharmaceutically acceptable salt, tautomer, or stereoisomer thereof, wherein
A is an albumin-binding moiety;
L is a linker;
X is —C═O or —CH₂;
Y is —O— or —CH₂; and
p is an integer from 0 to 1.

Described below are embodiments of the invention, where for convenience Embodiment 1 (E1) is identical to the embodiment of Formula (I) provided above.

It is to be understood that both the foregoing general description and the following detailed description are exemplary and explanatory only and are not restrictive of the invention, as claimed.

### DETAILED DESCRIPTION OF THE INVENTION

The compounds, combinations and methods of the present invention are believed to have one or more advantages, such as delivering the TLR7/8 adjuvant to the lymph node via albumin trafficking, which will have a sustained exposure profile in the lymph node over systemic circulation. This is important as systemically administered TLR7/8 agonists can lead to unwanted on mechanism toxicity. Retention of the adjuvant in the lymph node via albumin trafficking will provide sustained exposure and accompanied stimulation of the immune system to allow for optimal immune response to the vaccine antigen.

The present invention may be understood more readily by reference to the following detailed description of the embodiments of the invention and the Examples included herein. It is to be understood that this invention is not limited to specific synthetic methods of making that may of course vary. It is to be also understood that the terminology used herein is for the purpose of describing specific embodiments only and is not intended to be limiting.

E1 A compound of Formula (I) or a pharmaceutically acceptable salt thereof, as defined above.

E2 A compound of embodiment E1, wherein
A is cholesterol or tocopherol, and
further wherein
if A is cholesterol, p is 0, and
if A is tocopherol, p is 1.

E3 A compound of embodiment E2, wherein the tocopherol is selected from the group consisting of:
(i) alpha-tocopherol

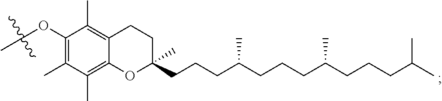 [SMILES: *Oc1c(C)c(C)c2c(c1C)CC[C@@](C)(CCC[C@H](C)CCC[C@H](C)CCCC(C)C)O2]

(ii) beta-tocopherol

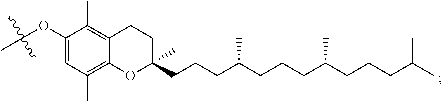 [SMILES: *Oc1cc(C)c2c(c1C)CC[C@@](C)(CCC[C@H](C)CCC[C@H](C)CCCC(C)C)O2]

(iii) gamma-tocopherol

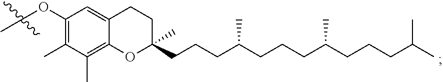 [SMILES: *Oc1cc2c(c(C)c1C)O[C@](C)(CCC[C@H](C)CCC[C@H](C)CCCC(C)C)CC2]

and
(iv) delta-tocopherol

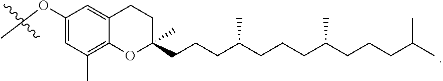 [SMILES: *Oc1cc(C)c2c(c1)CC[C@@](C)(CCC[C@H](C)CCC[C@H](C)CCCC(C)C)O2]

E4 A compound of embodiment E1 having Formula (Ia)

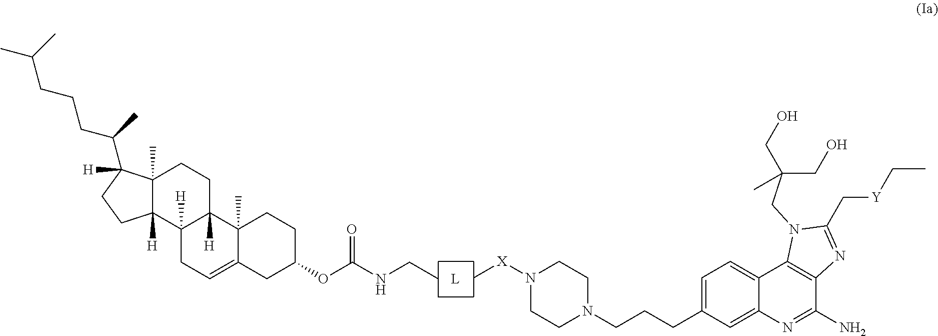

or a pharmaceutically acceptable salt, tautomer, or stereoisomer thereof, wherein
L is a linker;
X is —C═O or —CH₂; and
Y is —O— or —CH₂.

E5 A compound of embodiment E4, wherein L has the following structure:

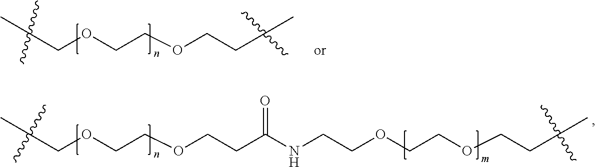

wherein
m is an integer from 1 to 35, and
n is an integer from 0 to 30.

E6 A compound of embodiment E5 having Formula (Ia(i))

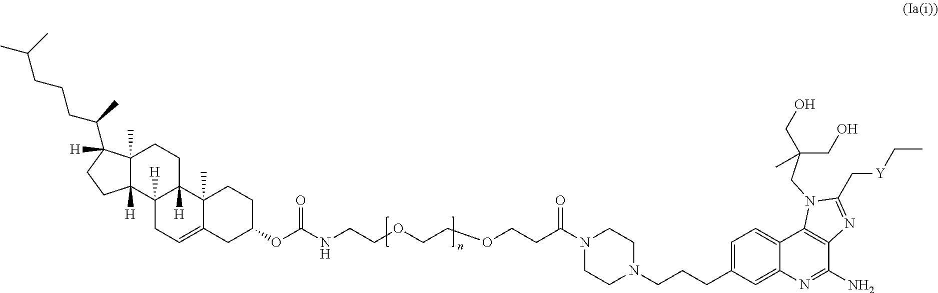

or a pharmaceutically acceptable salt, tautomer, or stereoisomer thereof, wherein
Y is —O— or —CH₂; and
n is an integer from 0 to 30.

E7 A compound of embodiment E5 having Formula (Ia(ii))

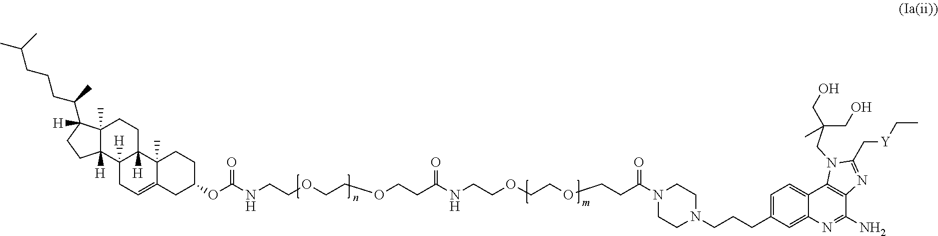

or a pharmaceutically acceptable salt, tautomer, or stereoisomer thereof, wherein
Y is —O— or —CH₂
m is an integer from 1 to 35, and
n is an integer from 0 to 30.

E8 A compound of embodiment E5 having Formula (Ia(iii))

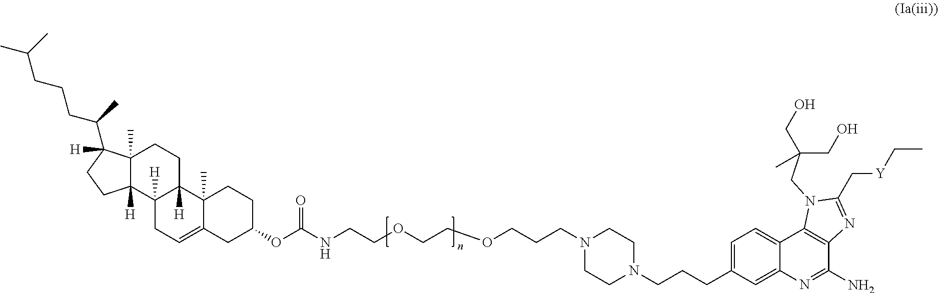

or a pharmaceutically acceptable salt, tautomer, or stereoisomer thereof, wherein
Y is —O— or —CH₂; and
n is an integer from 0 to 30.

E9 A compound of embodiment E5 having Formula (Ia(iv))

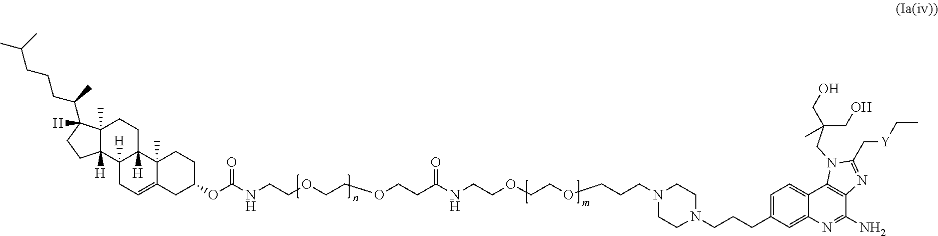

or a pharmaceutically acceptable salt, tautomer, or stereoisomer thereof, wherein
Y is —O— or —CH₂;
m is an integer from 1 to 35; and
n is an integer from 0 to 30.

E10 A compound of embodiment E2 having Formula (Ib)

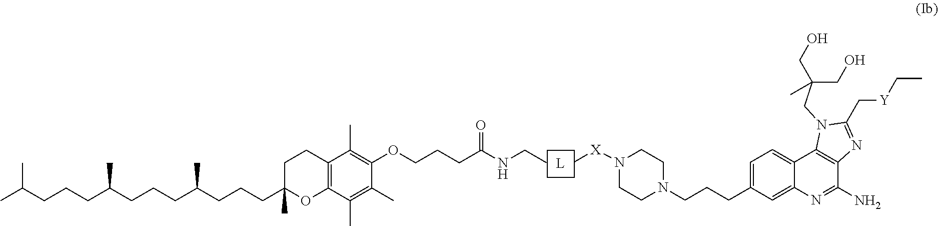

or a pharmaceutically acceptable salt, tautomer, or stereoisomer thereof, wherein
L is a linker;
X is —C═O or —CH₂; and
Y is —O— or —CH₂.

E11 A compound of embodiment E8, wherein L has the following structure:

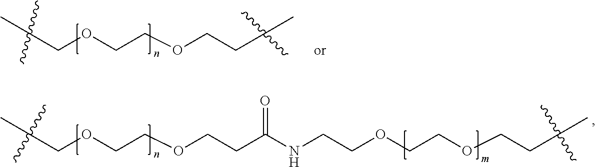

wherein
m is an integer from 1 to 35, and
n is an integer from 0 to 30.

E12 A compound of embodiment E11 having Formula (Ib(i))

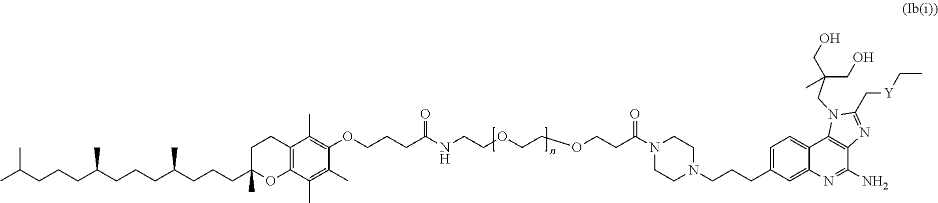

or a pharmaceutically acceptable salt, tautomer, or stereoisomer thereof, wherein
Y is —O— or —CH₂; and
n is an integer from 0 to 30.

E13 A compound of embodiment E11 having Formula (Ib(ii))

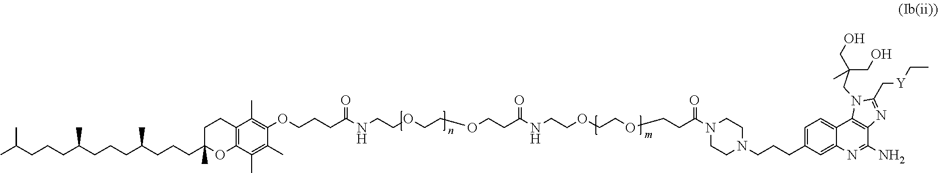

or a pharmaceutically acceptable salt, tautomer, or stereoisomer thereof, wherein
Y is —O— or —CH₂;
m is an integer from 1 to 35; and
n is an integer from 0 to 30.

E14 A compound of embodiment E11 having Formula (Ib(iii))

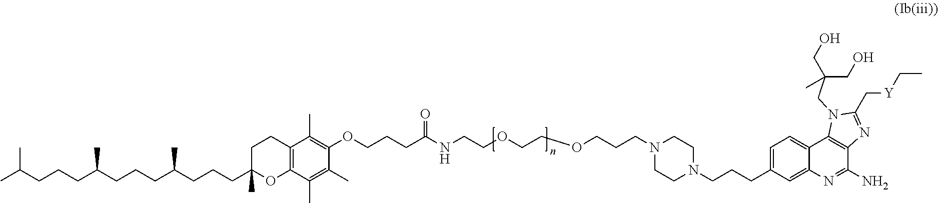

or a pharmaceutically acceptable salt, tautomer, or stereoisomer thereof, wherein
Y is —O— or —CH₂; and
n is an integer from 0 to 30.

E15 A compound of embodiment E11 having Formula (Ib(iv))

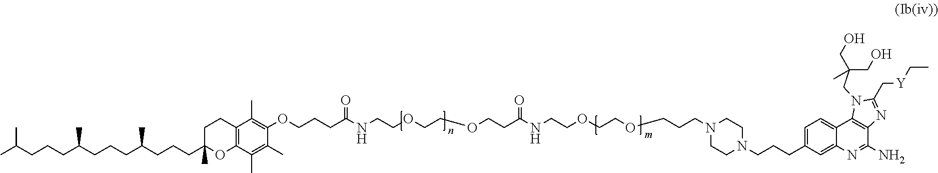

or a pharmaceutically acceptable salt, tautomer, or stereoisomer thereof, wherein
Y is —O— or —CH₂;
m is an integer from 1 to 35; and
n is an integer from 0 to 30.

E16 A compound of Formula (I)

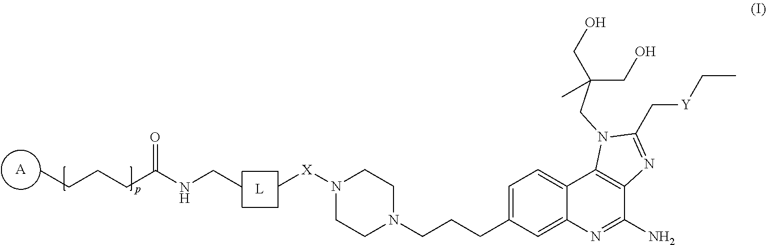

or a pharmaceutically acceptable salt, tautomer, or stereoisomer thereof, wherein
A is an albumin-binding moiety;
L is a linker;
X is C═O or CH₂;
Y is O or CH₂; and
p is an integer from 0 to 1.

E17 A compound of embodiment E16, wherein
A is cholesterol or tocopherol, and
further wherein
if A is cholesterol, p is 0, and
if A is tocopherol, p is 1.

E18 A compound of embodiment E17, wherein the tocopherol is selected from the group consisting of:
(i) alpha-tocopherol

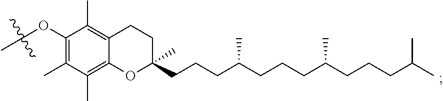 [SMILES: *Oc1c(C)c(C)c2c(c1C)CC[C@@](C)(CCC[C@H](C)CCC[C@H](C)CCCC(C)C)O2]

(ii) beta-tocopherol

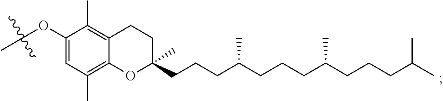 [SMILES: *Oc1cc(C)c2c(c1C)CC[C@@](C)(CCC[C@H](C)CCC[C@H](C)CCCC(C)C)O2]

(iii) gamma-tocopherol

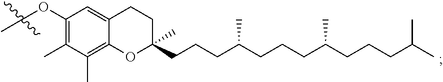 [SMILES: *Oc1cc2c(c(C)c1C)O[C@](C)(CCC[C@H](C)CCC[C@H](C)CCCC(C)C)CC2]

and
(iv) delta-tocopherol

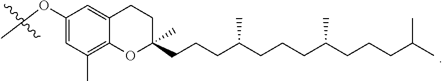 [SMILES: *Oc1cc(C)c2c(c1)CC[C@@](C)(CCC[C@H](C)CCC[C@H](C)CCCC(C)C)O2]

E19 A compound of embodiment E16 having Formula (Ia)

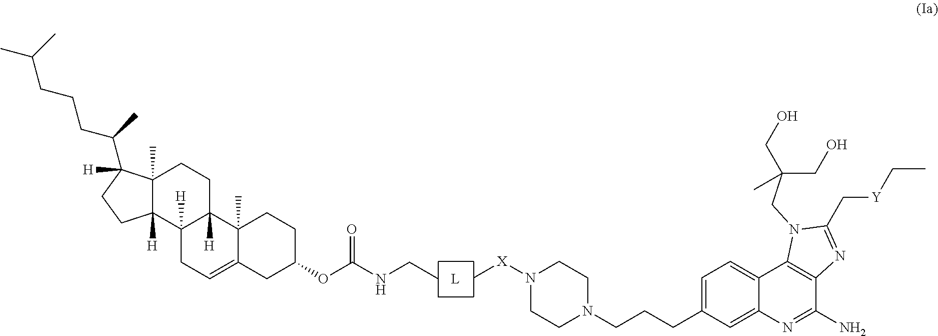

or a pharmaceutically acceptable salt, tautomer, or stereoisomer thereof, wherein
L is a linker;
X is C═O or CH₂; and
Y is O or CH₂.

E20 A compound of embodiment E19, wherein L has the following structure:

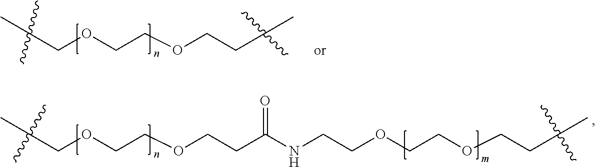

wherein
m is an integer from 1 to 35, and
n is an integer from 0 to 30.

E21 A compound of embodiment E20 having Formula (Ia(i))

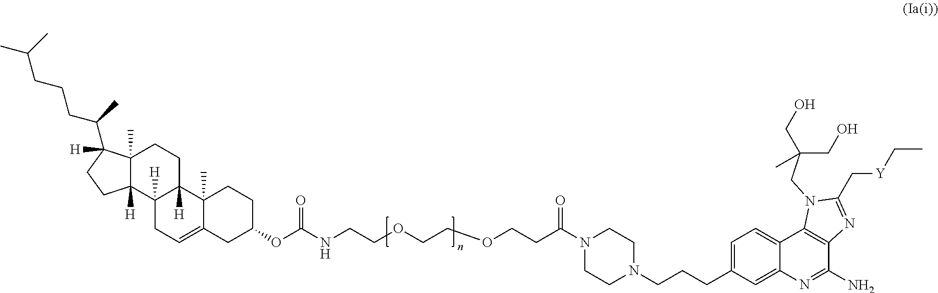

or a pharmaceutically acceptable salt, tautomer, or stereoisomer thereof, wherein
Y is O or CH₂; and
n is an integer from 0 to 30.

E22 A compound of embodiment E20 having Formula (Ia(ii))

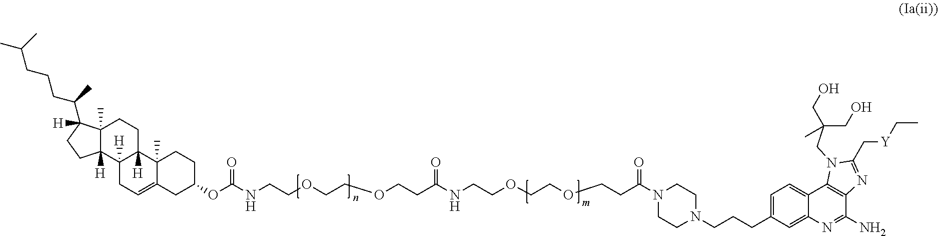

or a pharmaceutically acceptable salt, tautomer, or stereoisomer thereof, wherein
Y is O or CH₂;
m is an integer from 1 to 35; and
n is an integer from 0 to 30.

E23 A compound of embodiment E20 having Formula (Ia(iii))

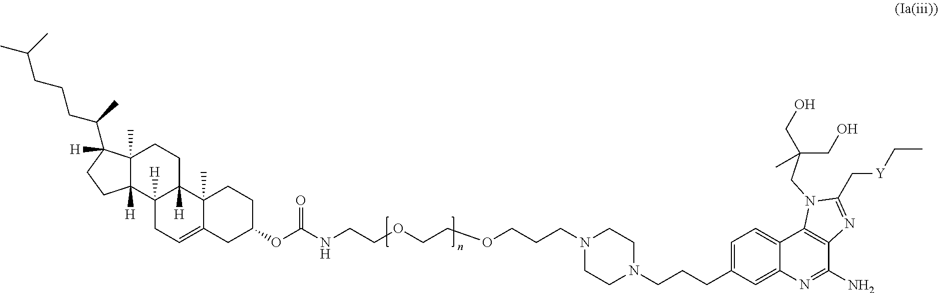

or a pharmaceutically acceptable salt, tautomer, or stereoisomer thereof, wherein
Y is O or CH₂; and
n is an integer from 0 to 30.

E24 A compound of embodiment E20 having Formula (Ia(iv))

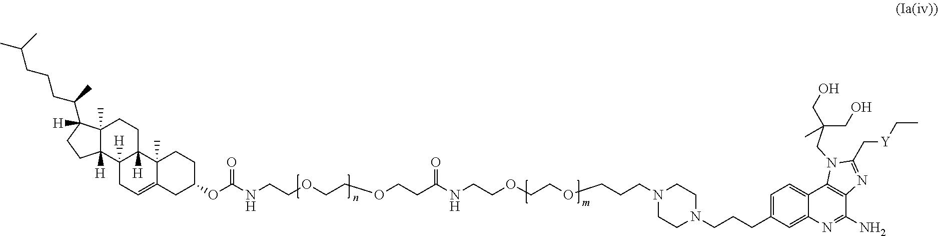

or a pharmaceutically acceptable salt, tautomer, or stereoisomer thereof, wherein
Y is O or CH₂;
m is an integer from 1 to 35; and
n is an integer from 0 to 30.

E25 A compound of embodiment E2 having Formula (Ib)

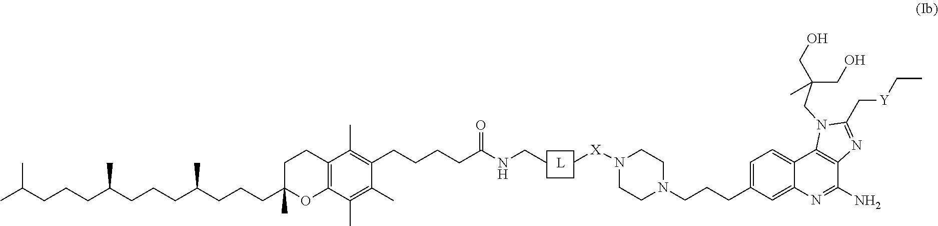

or a pharmaceutically acceptable salt, tautomer, or stereoisomer thereof, wherein
L is a linker;
X is C═O or CH₂; and
Y is O or CH₂.

E26 A compound of embodiment E25, wherein L has the following structure:

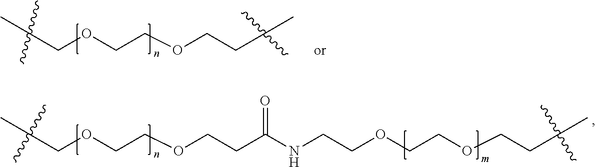

wherein
m is an integer from 1 to 35, and
n is an integer from 0 to 30.

E27 A compound of embodiment E26 having Formula (Ib(i))

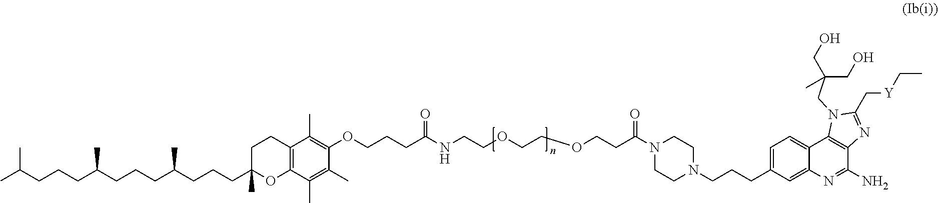

or a pharmaceutically acceptable salt, tautomer, or stereoisomer thereof, wherein
Y is O or CH₂; and
n is an integer from 0 to 30.

E28 A compound of embodiment E26 having Formula (Ib(ii))

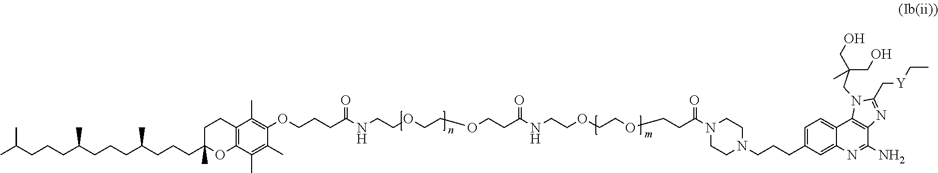

or a pharmaceutically acceptable salt, tautomer, or stereoisomer thereof, wherein
Y is O or CH₂;
m is an integer from 1 to 35; and
n is an integer from 0 to 30.

E29 A compound of embodiment E26 having Formula (Ib(iii))

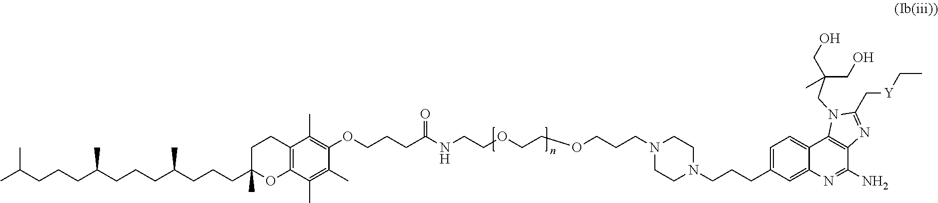

or a pharmaceutically acceptable salt, tautomer, or stereoisomer thereof, wherein
Y is O or CH₂; and
n is an integer from 0 to 30.

E30 A compound of embodiment E26 having Formula (Ib(iv))

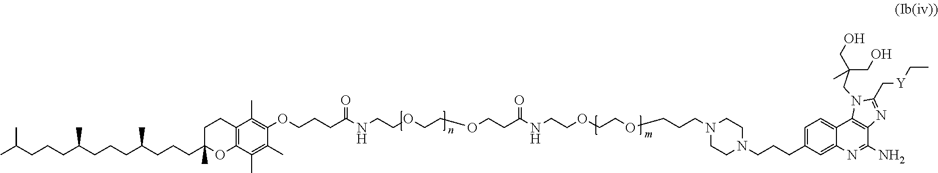

or a pharmaceutically acceptable salt, tautomer, or stereoisomer thereof, wherein
Y is O or CH₂;
m is an integer from 1 to 35; and
n is an integer from 0 to 30.

E31 A compound of any one of embodiments E16-E19, wherein L is a cleavage linker.

E32 A compound of any one of embodiments E16-E19 or E31, wherein L is a cleavage linker with an amide bond.

E33 A compound of any one of embodiments E16-E19 or E31-E32, wherein L has the following structure:
wherein

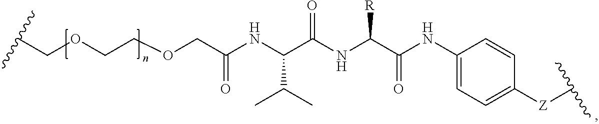

R is CH₃ or;

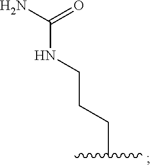 [SMILES: *CCCNC(N)=O]

and
Z is CH₂O.

E34 A compound of any one of embodiments E16-E19 or E31-E32, wherein L has the following structure:

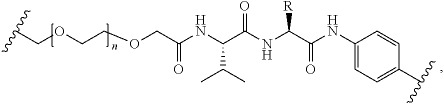

wherein
R is CH₃ or

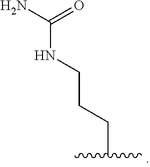 [SMILES: *CCCNC(N)=O]

E35 A compound of any one of embodiments E1-E34, wherein the compound is selected from the group consisting of:
(3beta)-cholest-5-en-3-yl {91-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-15,91-dioxo-3,6,9,12,19,22,25,28,31,34,37,40,43,46,49,52,55,58,61,64,67,70,73,76,79,82,85,88-octacosaoxa-16-azahennonacont-1-yl}carbamate;
N-{75-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-75-oxo-3,6,9,12,15,18,21,24,27,30,33,36,39,42,45,48,51,54,57,60,63,66,69,72-tetracosaoxapentaheptacont-1-yl}-17-oxo-20-({(2R)-2,5,7,8-tetramethyl-2-[(4R,8R)-4,8,12-trimethyltridecyl]-3,4-dihydro-2H-chromen-6-yl}oxy)-4,7,10,13-tetraoxa-16-azaicosan-1-amide;
(3beta)-cholest-5-en-3-yl {127-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-15,127-dioxo-3,6,9,12,19,22,25,28,31,34,37,40,43,46,49,52,55,58,61,64,67,70,73,76,79,82,85,88,91,94,97,100,103,106,109,112,115,118,121,124-tetracontaoxa-16-azaheptacosahect-1-yl}carbamate;
(3beta)-cholest-5-en-3-yl {127-[4-(3-{4-amino-2-(ethoxymethyl)-1-[3-hydroxy-2-(hydroxymethyl)-2-methylpropyl]-1H-imidazo[4,5-c]quinolin-7-yl}propyl)piperazin-1-yl]-15,127-dioxo-3,6,9,12,19,22,25,28,31,34,37,40,43,46,49,52,55,58,61,64,67,70,73,76,79,82,85,88,91,94,97,100,103,106,109,112,115,118,121,124-tetracontaoxa-16-azaheptacosahect-1-yl}carbamate;
(3beta)-cholest-5-en-3-yl {91-[4-(3-{4-amino-2-(ethoxymethyl)-1-[3-hydroxy-2-(hydroxymethyl)-2-methylpropyl]-1H-imidazo[4,5-c]quinolin-7-yl}propyl)piperazin-1-yl]-15,91-dioxo-3,6,9,12,19,22,25,28,31,34,37,40,43,46,49,52,55,58,61,64,67,70,73,76,79,82,85,88-octacosaoxa-16-azahennonacont-1-yl}carbamate;
(3beta,20R)-cholest-5-en-3-yl {55-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-15,55-dioxo-3,6,9,12,19,22,25,28,31,34,37,40,43,46,49,52-hexadecaoxa-16-azapentapentacont-1-yl}carbamate;
(3beta)-cholest-5-en-3-yl {63-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-3,6,9,12,15,18,21,24,27,30,33,36,39,42,45,48,51,54,57,60-icosaoxatrihexacont-1-yl}carbamate;
(3beta,20R)-cholest-5-en-3-yl {151-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-75,151-dioxo-3,6,9,12,15,18,21,24,27,30,33,36,39,42,45,48,51,54,57,60,63,66,69,72,79,82,85,88,91,94,97,100,103,106,109,112,115,118,121,124,127,130,133,136,139,142,145,148-octatetracontaoxa-76-azahenpentacontahect-1-yl}carbamate;
(3beta)-cholest-5-en-3-yl {75-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-75-oxo-3,6,9,12,15,18,21,24,27,30,33,36,39,42,45,48,51,54,57,60,63,66,69,72-tetracosaoxapentaheptacont-1-yl}carbamate;
(3beta,20R)-cholest-5-en-3-yl (2-{3-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]propoxy}ethyl)carbamate;
(3beta)-cholest-5-en-3-yl {167-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-15,91,167-trioxo-3,6,9,12,19,22,25,28,31,34,37,40,43,46,49,52,55,58,61,64,67,70,73,76,79,82,85,88,95,98,101,104,107,110,113,116,119,122,125,128,131,134,137,140,143,146,149,152,155,158,1 61,164-dopentacontaoxa-16,92-diazaheptahexacontahect-1-yl}carbamate;
(3beta,20R)-cholest-5-en-3-yl {15-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-15-oxo-3,6,9,12-tetraoxapentadec-1-yl}carbamate;
(3beta,20R)-cholest-5-en-3-yl {55-[4-(3-{4-amino-2-(ethoxymethyl)-1-[3-hydroxy-2-(hydroxymethyl)-2-methylpropyl]-1H-imidazo[4,5-c]quinolin-7-yl}propyl)piperazin-1-yl]-15,55-dioxo-3,6,9,12,19,22,25,28,31,34,37,40,43,46,49,52-hexadecaoxa-16-azapentapentacont-1-yl}carbamate;
(3beta,20R)-cholest-5-en-3-yl {51-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-3,6,9,12,15,18,21,24,27,30,33,36,39,42,45,48-hexadecaoxahenpentacont-1-yl}carbamate
(20R)-cholest-5-en-3beta-yl {31-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-15-oxo-3,6,9,12,19,22,25,28-octaoxa-16-azahentriacontan-1-yl}carbamate;
(20R)-cholest-5-en-3beta-yl {75-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-3,6,9,12,15,18,21,24,27,30,33,36,39,42,45,48,51,54,57,60,63,66,69,72-tetracosaoxapentaheptacontan-1-yl}carbamate;
(3beta)-cholest-5-en-3-yl {91-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-15-oxo-3,6,9,12,19,22,25,28,31,34,37,40,43,46,49,52,55,58,61,64,67,70,73,76,79,82,85,88-octacosaoxa-16-azahennonacont-1-yl}carbamate;
(20R)-cholest-5-en-3beta-yl [2-(2-{3-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]propoxy}ethoxy)ethyl]carbamate;
(20R)-cholest-5-en-3beta-yl {27-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-27-oxo-3,6,9,12,15,18,21,24-octaoxaheptacosan-1-yl}carbamate;
(20R)-cholest-5-en-3beta-yl {39-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-39-oxo-3,6,9,12,15,18,21,24,27,30,33,36-dodecaoxanonatriacontan-1-yl}carbamate;
(20R)-cholest-5-en-3beta-yl {39-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-3,6,9,12,15,18,21,24,27,30,33,36-dodecaoxanonatriacontan-1-yl}carbamate;
(20R)-cholest-5-en-3beta-yl {15-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-3,6,9,12-tetraoxapentadecan-1-yl}carbamate;
(20R)-cholest-5-en-3beta-yl {27-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-3,6,9,12,15,18,21,24-octaoxaheptacosan-1-yl}carbamate;
N-{111-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-111-oxo-3,6,9,12,15,18,21,24,27,30,33,36,39,42,45,48,51,54,57,60,63,66,69,72,75,78,81,84,87,90,93,96,99,102,105,108-hexatriacontaoxahendecahectan-1-yl}-17-oxo-20-({(2R)-2,5,7,8-tetramethyl-2-[(4R,8R)-4,8,12-trimethyltridecyl]-3,4-dihydro-2H-1-benzopyran-6-yl}oxy)-4,7,10,13-tetraoxa-16-azaicosan-1-amide;
N-{39-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-39-oxo-3,6,9,12,15,18,21,24,27,30,33,36-dodecaoxanonatriacontan-1-yl}-17-oxo-20-({(2R)-2,5,7,8-tetramethyl-2-[(4R,8R)-4,8,12-trimethyltridecyl]-3,4-dihydro-2H-1-benzopyran-6-yl}oxy)-4,7,10,13-tetraoxa-16-azaicosan-1-amide;
N-{75-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-3,6,9,12,15,18,21,24,27,30,33,36,39,42,45,48,51,54,57,60,63,66,69,72-tetracosaoxapentaheptacontan-1-yl}-17-oxo-20-({(2R)-2,5,7,8-tetramethyl-2-[(4R,8R)-4,8,12-trimethyltridecyl]-3,4-dihydro-2H-1-benzopyran-6-yl}oxy)-4,7,10,13-tetraoxa-16-azaicosan-1-amide;
(3beta)-cholest-5-en-3-yl {(2S,5S)-1-((4-((4-(3-(4-amino-2-butyl-1-(3-hydroxy-2-(hydroxymethyl)-2-methylpropyl)-1H-imidazo[4,5-c]quinolin-7-yl)propyl)piperazin-1-yl)methyl)phenyl)amino)-5-isopropyl-2-methyl-1,4,7-trioxo-9,12-dioxa-3,6-diazatetradecan-14-yl}carbamate;
4-((12S,15S)-1-(((3beta)-cholest-5-en-3-yl)oxy)-12-isopropyl-1,10,13-trioxo-15-(3-ureidopropyl)-5,8-dioxa-2,11,14-triazahexadecan-16-amido)benzyl 4-(3-(4-amino-2-butyl-1-(3-hydroxy-2-(hydroxymethyl)-2-methylpropyl)-1H-imidazo[4,5-c]quinolin-7-yl)propyl)piperazine-1-carboxylate; and
(3beta)-cholest-5-en-3-yl ((6S,9S)-1-amino-6-((4-((4-(3-(4-amino-2-butyl-1-(3-hydroxy-2-(hydroxymethyl)-2-methylpropyl)-1H-imidazo[4,5-c]quinolin-7-yl)propyl)piperazin-1-yl)methyl)phenyl)carbamoyl)-9-isopropyl-1,8,11-trioxo-13,16-dioxa-2,7,10-triazaoctadecan-18-yl)carbamate;
or a pharmaceutically acceptable salt thereof.

E36 A compound, which is (3beta)-cholest-5-en-3-yl {91-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-15,91-dioxo-3,6,9,12,19,22,25,28,31,34,37,40,43,46,49,52,55,58,61,64,67,70,73,76,79,82,85,88-octacosaoxa-16-azahennonacont-1-yl}carbamate, or a pharmaceutically acceptable salt thereof.

E37 A compound, which is N-{75-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-75-oxo-3,6,9,12,15,18,21,24,27,30,33,36,39,42,45,48,51,54,57,60,63,66,69,72-tetracosaoxapentaheptacont-1-yl}-17-oxo-20-({(2R)-2,5,7,8-tetramethyl-2-[(4R,8R)-4,8,12-trimethyltridecyl]-3,4-dihydro-2H-chromen-6-yl}oxy)-4,7,10,13-tetraoxa-16-azaicosan-1-amide, or a pharmaceutically acceptable salt thereof.

E38 A pharmaceutically acceptable salt of (3beta)-cholest-5-en-3-yl {91-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-15,91-dioxo-3,6,9,12,19,22,25,28,31,34,37,40,43,46,49,52,55,58,61,64,67,70,73,76,79,82,85,88-octacosaoxa-16-azahennonacont-1-yl}carbamate.

E39 A pharmaceutically acceptable salt of N-{75-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-75-oxo-3,6,9,12,15,18,21,24,27,30,33,36,39,42,45,48,51,54,57,60,63,66,69,72-tetracosaoxapentaheptacont-1-yl}-17-oxo-20-({(2R)-2,5,7,8-tetramethyl-2-[(4R,8R)-4,8,12-trimethyltridecyl]-3,4-dihydro-2H-chromen-6-yl}oxy)-4,7,10,13-tetraoxa-16-azaicosan-1-amide.

E40 A compound, which is (3beta)-cholest-5-en-3-yl {91-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-15,91-dioxo-3,6,9,12,19,22,25,28,31,34,37,40,43,46,49,52,55,58,61,64,67,70,73,76,79,82,85,88-octacosaoxa-16-azahennonacont-1-yl}carbamate.

E41 A compound, which is N-{75-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-75-oxo-3,6,9,12,15,18,21,24,27,30,33,36,39,42,45,48,51,54,57,60,63,66,69,72-tetracosaoxapentaheptacont-1-yl}-17-oxo-20-({(2R)-2,5,7,8-tetramethyl-2-[(4R,8R)-4,8,12-trimethyltridecyl]-3,4-dihydro-2H-chromen-6-yl}oxy)-4,7,10,13-tetraoxa-16-azaicosan-1-amide.

E42 A pharmaceutical composition comprising the compound according to any of embodiments E1 to E41, or a pharmaceutically acceptable salt thereof, and at least one pharmaceutically acceptable excipient.

E43 A method of inducing an immune response to an antigen of interest in a subject, comprising administering to the subject the pharmaceutical composition of embodiment E42 and an immunogenic composition comprising the antigen of interest.

E44 A method for immunizing a subject against a disease or disorder caused by or associated with an antigen of interest, comprising administering to the subject the pharmaceutical composition of embodiment E42 and an immunogenic composition comprising the antigen of interest.

E45 A method for preventing a disease or disorder caused by or associated with an antigen of interest in a subject, comprising administering to the subject the pharmaceutical composition of embodiment E42 and an immunogenic composition comprising the antigen of interest.

E46 A method for treating a disease or disorder caused by or associated with an antigen of interest in a subject, comprising administering to the subject the pharmaceutical composition of embodiment E42 and an immunogenic composition comprising the antigen of interest.

E47 A method for increasing an immune response to an antigen of interest in a subject, comprising administering to the subject the pharmaceutical composition of embodiment E42 and an immunogenic composition comprising the antigen of interest.

E48 The method of any of embodiments E43 to E47, wherein the antigen of interest is an infectious disease antigen.

E49 The method of any of embodiments E43 to E48, wherein the antigen of interest is a viral antigen, a bacterial antigen, a fungal antigen, or a parasitic antigen.

E50 The method of E49, wherein the antigen of interest is a Streptococcus pneumoniae polysaccharide.

E51 The method of E49, wherein the antigen of interest is Streptococcus pneumoniae serotype 3 polysaccharide.

E52 The method of E49, wherein the antigen of interest is a conjugated Streptococcus pneumoniae serotype 3 polysaccharide.

E53 The method of E52, wherein the Streptococcus pneumoniae serotype 3 polysaccharide is conjugated to CRM₁₉₇ or streptococcal C5a peptidase (SCP).

E54 The method of E49, wherein the antigen of interest is a RSV protein or RNA encoding a RSV protein.

E55 The method of E49 or E54, wherein the antigen of interest is a RSV F protein or RNA encoding a RSV F protein.

E56 The method of E49 or E54-E55, wherein the antigen of interest is a RSV F protein of subtype A, or RNA encoding a RSV F protein of subtype A, and a RSV F protein of subtype B, or RNA encoding a RSV F protein of subtype B.

E57 The method of any one of claims E55-E56, wherein the RSV F protein(s) comprise the mutations T103C, I148C, S190I, and D486S.

E58 The method of E49 or E54-E57, wherein the antigen of interest is a RSV protein comprising the sequence of SEQ ID NO: 1 and SEQ ID NO: 2 or a RSV protein comprising the sequence of SEQ ID NO: 3 and SEQ ID NO: 4.

E59 The method of E49 or E54-E58, wherein the antigen of interest is bivalent RSV and comprises a RSV protein comprising the sequence of SEQ ID NO: 1 and SEQ ID NO: 2 and a RSV protein comprising the sequence of SEQ ID NO: 3 and SEQ ID NO: 4.

E60 The method of any of embodiments E43 to E47, wherein the antigen of interest is a cancer antigen.

E61 The method of any one of embodiments E43 to E60, wherein the pharmaceutical composition and the immunogenic composition are administered to the subject concurrently.

E62 The method of any one of embodiments E43 to E60, wherein the pharmaceutical composition is administered to the subject prior to the administration of the immunogenic composition.

E63 The method of any one of embodiments E43 to E60, wherein the pharmaceutical composition is administered to the subject after the administration of the immunogenic composition.

E64 The method of any one of embodiments E43 to E63, wherein the pharmaceutical composition and immunogenic composition are administered to the subject by the same route of administration.

E65 The method of embodiment E64, wherein the route of administration is subcutaneous or intramuscular.

E66 The method of any one of embodiments E43 to E60 or E62-E63, wherein the pharmaceutical composition and the immunogenic composition are administered to the subject by different routes of administration.

E67 The method of any one of embodiments E43 to E66, wherein the pharmaceutical composition and the immunogenic composition are administered to the same region of the subject.

E68 The method of any one of embodiments E43-E67, wherein the immune response to the antigen of interest is at least 10%, at least 25%, at least 30%, at least 40% or at least 50% higher than the immune response to the antigen of interest without the administration of the compound according to any of embodiments E1 to E41 or the pharmaceutical composition of embodiment E42.

E69 The method of embodiment E68, wherein the immune response is a humoral immune response.

E70 The method of embodiment E68, wherein the immune response is a cellular immune response.

E71 The method of any one of embodiments E43 to E70, wherein the subject is human.

E72 A compound according to any of embodiments E1 to E41 for use as a medicament.

E73 A compound according to any of embodiments E1 to E41 for use in inducing an immune response to an antigen of interest in a subject.

E74 A compound according to any of embodiments E1 to E41 for use in immunizing a subject against a disease or disorder caused by or associated with an antigen of interest in a subject.

E75 A compound according to any of embodiments E1 to E41 for use in preventing a disease or disorder caused by or associated with an antigen of interest in a subject.

E76 A compound according to any of embodiments E1 to E41 for use in treating a disease or disorder caused by or associated with an antigen of interest in a subject.

E77 A compound according to any of embodiments E1 to E41 for use in increasing an immune response to an antigen of interest in a subject.

E78 The compound according to any one of embodiments E73-77, wherein the subject is a human.

E79 Use of a compound according to any of embodiments E1 to E41 for the manufacture of a medicament for inducing an immune response to an antigen of interest in a subject.

E80 Use of a compound according to any of embodiments E1 to E41 for the manufacture of a medicament for immunizing a subject against a disease or disorder caused by or associated with an antigen of interest.

E81 Use of a compound according to any of embodiments E1 to E41 for the manufacture of a medicament for use in preventing a disease or disorder caused by or associated with an antigen of interest in the subject.

E82 Use of a compound according to any of embodiments E1 to E41 for the manufacture of a medicament for treating a disease or disorder caused by or associated with an antigen of interest in the subject.

E83 Use of a compound according to any of embodiments E1 to E41 for the manufacture of a medicament for increasing an immune response to an antigen of interest in a subject.

E84 The use according to any one of embodiments E79 to E83, wherein the subject is a human.

E75 A crystal comprising the compound according to any of embodiments E1 to E41 or a pharmaceutically acceptable salt thereof.

Furthermore, each of the embodiments described herein envisions within its scope pharmaceutically acceptable salts of the compounds described herein.

### Definitions

Unless otherwise defined herein, scientific and technical terms used in connection with the present invention have the meanings that are commonly understood by those of ordinary skill in the art.

The invention described herein suitably may be practiced in the absence of any element(s) not specifically disclosed herein.

“Compounds of the invention” include compounds of Formula (I), (Ia), (Ia(i)), (Ia(ii)), (Ia(iii)), (Ia(iv)), (Ib), (Ib(i)), (Ib(ii)), (Ib(iii)), (Ib(iv)) and the novel intermediates used in the preparation thereof. One of ordinary skill in the art will appreciate that compounds of the invention include conformational isomers (e.g., cis and trans isomers) and all optical isomers (e.g., enantiomers and diastereomers), racemic, diastereomeric and other mixtures of such isomers, tautomers thereof, where they may exist. One of ordinary skill in the art will also appreciate that compounds of the invention include solvates, hydrates, isomorphs, polymorphs, esters, salt forms, prodrugs, derivatives and isotopically labeled versions thereof, where they may be formed.

As used herein, the singular form “a”, “an”, and “the” include plural references unless indicated otherwise. For example, “a” substituent includes one or more substituents.

As used herein, the term “about” when used to modify a numerically defined parameter (e.g., the dose of XXX) means that the parameter may vary by as much as 10% below or above the stated numerical value for that parameter. For example, a dose of about 5 mg means 5 mg±10%, i.e., it may vary between 4.5 mg and 5.5 mg.

If substituents are described as being “independently selected” from a group, each substituent is selected independent of the other. Each substituent therefore may be identical to or different from the other substituent(s).

“Optional” or “optionally” means that the subsequently described event or circumstance may, but need not occur, and the description includes instances where the event or circumstance occurs and instances in which it does not.

The terms “optionally substituted” and “substituted or unsubstituted” are used interchangeably to indicate that the particular group being described may have no non-hydrogen substituents (i.e., unsubstituted), or the group may have one or more non-hydrogen substituents (i.e., substituted). If not otherwise specified, the total number of substituents that may be present is equal to the number of H atoms present on the unsubstituted form of the group being described. Where an optional substituent is attached via a double bond, such as an oxo (═O) substituent, the group occupies two available valences, so the total number of other substituents that are included is reduced by two. In the case where optional substituents are selected independently from a list of alternatives, the selected groups may be the same or different. Throughout the disclosure, it will be understood that the number and nature of optional substituent groups will be limited to the extent that such substitutions make chemical sense to one of ordinary skill in the art.

“Hydroxy” refers to an —OH group.

“Oxo” refers to a double bonded oxygen (═O).

“Alkyl” refers to a saturated, monovalent aliphatic hydrocarbon radical that has a specified number of carbon atoms, including straight chain or branched chain groups. Alkyl groups may contain, but are not limited to, 1 to 12 carbon atoms (“C₁-C₁₂ alkyl”), 1 to 8 carbon atoms (“C₁-C₈ alkyl”), 1 to 6 carbon atoms (“C₁-C₆ alkyl”), 1 to 5 carbon atoms (“C₁-C₅ alkyl”), 1 to 4 carbon atoms (“C₁-C₄ alkyl”), 1 to 3 carbon atoms (“C₁-C₃ alkyl”), or 1 to 2 carbon atoms (“C₁-C₂ alkyl”). Examples include, but are not limited to, methyl, ethyl, n-propyl, isopropyl, n-butyl, sec-butyl, isobutyl, tert-butyl, n-pentyl, isopentyl, neopentyl, n-hexyl, n-heptyl, n-octyl, and the like. Alkyl groups may be optionally substituted, unsubstituted or substituted, as further defined herein.

“Alkoxy” refers to an alkyl group, as defined herein, that is single bonded to an oxygen atom. The attachment point of an alkoxy radical to a molecule is through the oxygen atom. An alkoxy radical may be depicted as alkyl-O—. Alkoxy groups may contain, but are not limited to, 1 to 8 carbon atoms (“C₁-C₈ alkoxy”), 1 to 6 carbon atoms (“C₁-C₆ alkoxy”), 1 to 4 carbon atoms (“C₁-C₄ alkoxy”), or 1 to 3 carbon atoms (“C₁-C₃ alkoxy”). Alkoxy groups include, but are not limited to, methoxy, ethoxy, n-propoxy, isobutoxy, and the like.

“Alkoxyalkyl” refers to an alkyl group, as defined herein, that is substituted by an alkoxy group, as defined herein. Examples include, but are not limited to, CH₃OCH₂— and CH₃CH₂OCH₂—.

“Alkenyl” refers to an alkyl group, as defined herein, consisting of at least two carbon atoms and at least one carbon-carbon double bond. For example, as used herein, the term “C₂-C₆ alkenyl” means straight or branched chain unsaturated radicals of 2 to 6 carbon atoms, including, but not limited to, ethenyl, 1-propenyl, 2-propenyl, 1-, 2-, or 3-butenyl, and the like.

“Alkynyl” refers to an alkyl group, as defined herein, consisting of at least two carbon atoms and at least one carbon-carbon triple bond. Examples include, but are not limited to, ethynyl, 1-propynyl, 2-propynyl, 1-, 2-, or 3-butynyl, and the like.

“Cycloalkyl” refers to a fully saturated hydrocarbon ring system that has the specified number of carbon atoms, which may be a monocyclic, bridged or fused bicyclic or polycyclic ring system that is connected to the base molecule through a carbon atom of the cycloalkyl ring. Cycloalkyl groups may contain, but are not limited to, 3 to 12 carbon atoms (“C₃-C₁₂ cycloalkyl”), 3 to 8 carbon atoms (“C₃-C₈ cycloalkyl”), 3 to 6 carbon atoms (“C₃-C₆ cycloalkyl”), 3 to 5 carbon atoms (“C₃-C₅ cycloalkyl”) or 3 to 4 carbon atoms (“C₃-C₄ cycloalkyl”). Examples include, but are not limited to, cyclopropyl, cyclobutyl, cyclopentyl, cyclohexyl, cycloheptyl, adamantanyl, and the like. Cycloalkyl groups may be optionally substituted, unsubstituted or substituted, as further defined herein.

“Cycloalkoxy” refers to a cycloalkyl group, as defined herein, that is single bonded to an oxygen atom. The attachment point of a cycloalkoxy radical to a molecule is through the oxygen atom. A cycloalkoxy radical may be depicted as cycloalkyl-O—. Cycloalkoxy groups may contain, but are not limited to, 3 to 8 carbon atoms (“C₃-C₈ cycloalkoxy”), 3 to 6 carbon atoms (“C₃-C₆ cycloalkoxy”), and 3 to 4 carbon atoms (“C₃-C₄ cycloalkoxy”). Cycloalkoxy groups include, but are not limited to, cyclopropoxy, cyclobutoxy, cyclopentoxy and the like.

“Amino” refers to a group —NH₂, which is unsubstituted. Where the amino is described as substituted or optionally substituted, the term includes groups of the form —NRxRy, where each of Rx and Ry is defined as further described herein. For example, “alkylamino” refers to a group —NRxRy, wherein one of Rx and Ry is an alkyl moiety and the other is H, and “dialkylamino” refers to —NRxRy wherein both of Rx and Ry are alkyl moieties, where the alkyl moieties have the specified number of carbon atoms (e.g., —NH(C₁-C₄ alkyl) or —N(C₁-C₄ alkyl)₂).

“Aminoalkyl” refers to an alkyl group, as defined above, that is substituted by 1, 2, or 3 amino groups, as defined herein.

The term “pharmaceutically acceptable” means the substance (e.g., the compounds described herein) and any salt thereof, or composition containing the substance or salt of the invention is suitable for administration to a subject or patient.

The compounds of the invention have asymmetric carbon atoms. The carbon-carbon bonds of the compounds of the invention may be depicted herein using a solid line ( [symbol] ), a solid wedge ( [symbol] ), or a dotted wedge ( [symbol] ). The use of a solid line to depict bonds to asymmetric carbon atoms is meant to indicate that all possible stereoisomers (e.g., specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of Formula (I) may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of Formula (I) can exist as enantiomers and diastereomers or as racemates and mixtures thereof. The use of a solid line to depict bonds to one or more asymmetric carbon atoms in a compound of Formula (I) and the use of a solid or dotted wedge to depict bonds to other asymmetric carbon atoms in the same compound is meant to indicate that a mixture of diastereomers is present.

### Salts

Salts encompassed within the term “pharmaceutically acceptable salts” refer to the compounds of this invention which are generally prepared by reacting the free base or free acid with a suitable organic or inorganic acid, or a suitable organic or inorganic base, respectively, to provide a salt of the compound of the invention that is suitable for administration to a subject or patient.

In addition, the compounds of Formula I may also include other salts of such compounds which are not necessarily pharmaceutically acceptable salts, which may be useful as intermediates for one or more of the following: 1) preparing compounds of Formula I; 2) purifying compounds of Formula I; 3) separating enantiomers of compounds of Formula I; or 4) separating diastereomers of compounds of Formula I.

Suitable acid addition salts are formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinofoate salts.

Suitable base salts are formed from bases which form non-toxic salts. Examples include, but are not limited to aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see Paulekun, G. S. et al., Trends in Active Pharmaceutical Ingredient Salt Selection Based on Analysis of the Orange Book Database, J. Med. Chem. 2007; 50(26), 6665-6672.

Pharmaceutically acceptable salts of compounds of the invention may be prepared by methods well known to one skilled in the art, including but not limited to the following procedures
(i) by reacting a compound of the invention with the desired acid or base;
(ii) by removing an acid- or base-labile protecting group from a suitable precursor of a compound of the invention or by ring-opening a suitable cyclic precursor, for example, a lactone or lactam, using the desired acid or base; or
(iii) by converting one salt of a compound of the invention to another. This may be accomplished by reaction with an appropriate acid or base or by means of a suitable ion exchange procedure.
   These procedures are typically carried out in solution. The resulting salt may precipitate out and be collected by filtration or may be recovered by evaporation of the solvent.

### Solvates

The compounds of the invention, and pharmaceutically acceptable salts thereof, may exist in unsolvated and solvated forms. The term ‘solvate’ is used herein to describe a molecular complex comprising the compound of the invention, or a pharmaceutically acceptable salt thereof, and one or more pharmaceutically acceptable solvent molecules, for example, ethanol. The term ‘hydrate’ is employed when said solvent is water.

In addition, the compounds of Formula I may also include other solvates of such compounds which are not necessarily pharmaceutically acceptable solvates, which may be useful as intermediates for one or more of the following: 1) preparing compounds of Formula I; 2) purifying compounds of Formula I; 3) separating enantiomers of compounds of Formula I; or 4) separating diastereomers of compounds of Formula I.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

### Complexes

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, for example, hydrogen bonded complex (cocrystal) may be formed with either a neutral molecule or with a salt. Co-crystals may be prepared by melt crystallization, by recrystallization from solvents, or by physically grinding the components together—see Chem Commun, 17; 1889-1896, by O. Almarsson and M. J. Zaworotko (2004). For a general review of multi-component complexes, see J Pharm Sci, 64(8), 1269-1288, by Haleblian (August 1975).

### Solid Form

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution) and consists of two dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

### Stereoisomers

Compounds of the invention may exist as two or more stereoisomers. Stereoisomers of the compounds may include cis and trans isomers (geometric isomers), optical isomers such as R and S enantiomers, diastereomers, rotational isomers, atropisomers, and conformational isomers. For example, compounds of the invention containing one or more asymmetric carbon atoms may exist as two or more stereoisomers. Where a compound of the invention contains an alkenyl or alkenylene group, geometric cis/trans (or Z/E) isomers are possible. Cis/trans isomers may also exist for saturated rings.

The pharmaceutically acceptable salts of compounds of the invention may also contain a counterion which is optically active (e.g., d-lactate or l-lysine) or racemic (e.g. dl-tartrate or dl-arginine).

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where a compound of the invention contains an acidic or basic moiety, a base or acid such as 1-phenylethylamine or tartaric acid. The resulting diastereomeric mixture may be separated by chromatography, fractional crystallization, or by using both of said techniques, and one or both of the diastereoisomers converted to the corresponding pure enantiomer(s) by means well known to a skilled person. Chiral compounds of the invention (and chiral precursors thereof) may be obtained in enantiomerically-enriched form using chromatography, typically HPLC Concentration of the eluate affords the enriched mixture. Chiral chromatography using sub-and supercritical fluids may be employed. Methods for chiral chromatography useful in some embodiments of the present invention are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein).

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

### Tautomerism

Where structural isomers are interconvertible via a low energy barrier, tautomeric isomerism (‘tautomerism’) may occur. This may take the form of proton tautomerism in compounds of the invention containing, for example, an imino/amino, keto/enol, or oxime/nitroso group, lactam/lactim or so-called valence tautomerism in compounds which contain an aromatic moiety. It follows that a single compound may exhibit more than one type of isomerism.

It must be emphasized that while, for conciseness, the compounds of the invention have been drawn herein in a single tautomeric form, all possible tautomeric forms are included within the scope of the invention.

### Isotopes

The present invention includes all pharmaceutically acceptable isotopically-labeled compounds of the invention wherein one or more atoms are replaced by atoms having the same atomic number, but an atomic mass or mass number different from the atomic mass or mass number which predominates in nature.

Examples of isotopes suitable for inclusion in the compounds of the invention may include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labeled compounds of the invention, for example those incorporating a radioactive isotope, are useful in one or both of drug or substrate tissue distribution studies. The radioactive isotopes tritium, i.e., ³H, and carbon-14, i.e., ¹⁴C, are particularly useful for this purpose in view of their ease of incorporation and ready means of detection.

Substitution with deuterium, i.e., ²H, may afford certain therapeutic advantages resulting from greater metabolic stability.

Substitution with positron emitting isotopes, such as ¹¹C, ¹⁸F, ¹⁵O and ¹³N, may be useful in Positron Emission Topography (PET) studies for examining substrate receptor occupancy.

Isotopically-labeled compounds of the invention may generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g., D₂O, d₆-acetone, d₆-DMSO.

### Metabolites

Also included within the scope of the invention are active metabolites of compounds of the invention, that is, compounds formed in vivo upon administration of the drug, often by oxidation or dealkylation. Some examples of metabolites in accordance with the invention include, but are not limited to,
where the compound of the invention contains an alkyl group, a hydroxyalkyl derivative thereof (—CH>—COH):
(ii) where the compound of the invention contains an alkoxy group, a hydroxy derivative thereof (—OR->—OH);
(iii) where the compound of the invention contains a tertiary amino group, a secondary amino derivative thereof (—NRR->—NHR or —NHR);
(iv) where the compound of the invention contains a secondary amino group, a primary derivative thereof (—NHR->—NH₂);
(v) where the compound of the invention contains a phenyl moiety, a phenol derivative thereof (-Ph->-PhOH);
(vi) where the compound of the invention contains an amide group, a carboxylic acid derivative thereof (—CONH₂->COOH); and
(vii) where the compound contains a hydroxy or carboxylic acid group, the compound may be metabolized by conjugation, for example with glucuronic acid to form a glucuronide. Other routes of conjugative metabolism exist. These pathways are frequently known as Phase 2 metabolism and include, for example, sulfation or acetylation. Other functional groups, such as NH groups, may also be subject to conjugation.

### Linkers

In some embodiments, the linker in the TLR 7/8 agonist molecules disclosed herein is a polyethylene glycol (PEG) linker. In some embodiments, the linker in the TLR 7/8 agonist molecules disclosed herein is a mono-dispersed PEG linker. In some embodiments, the linker in the TLR 7/8 agonist molecules disclosed herein is a polyethylene glycol (PEG) linker wherein the number of PEG repeats in the linker structure is an integer between about 1 and about 100. In some embodiments, the linker in the TLR 7/8 agonist molecules disclosed herein is a PEG linker wherein the number of PEG repeats in the linker structure is an integer between about 1 and about 35. In some embodiments, the linker in the TLR 7/8 agonist molecules disclosed herein is a PEG linker wherein the number of PEG repeats in the linker structure is an integer between about 1 and about 65. For example, in some embodiments, the number of PEG repeats in the linker structure is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65.

In some embodiments, the linker in the TLR 7/8 agonist molecules disclosed herein is a cleavage linker. In some embodiments, the linker in the TLR 7/8 agonist molecules disclosed herein is a cleavage linker comprising an amide, azide, carbamate, disulfide, ester, or peptide bond. In a particular embodiment, the linker in the TLR 7/8 agonist molecules disclosed herein is a cleavage linker comprising an amide bond.

### Pharmaceutical Compositions

In another embodiment, the invention comprises pharmaceutical compositions. For pharmaceutical composition purposes, the compound per se or pharmaceutically acceptable salt thereof will simply be referred to as the compounds of the invention.

A “pharmaceutical composition” refers to a mixture of one or more of the compounds of the invention, or a pharmaceutically acceptable salt, solvate or hydrate thereof as an active ingredient, and at least one pharmaceutically acceptable excipient.

The term ‘excipient’ is used herein to describe any ingredient other than the compound(s) of the invention. The choice of excipient will to a large extent depend on factors such as the mode of administration, the effect of the excipient on solubility and stability, and the nature of the dosage form.

As used herein, “excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives, or buffers, which enhance the shelf life or effectiveness of the compound.

The compositions of this invention may be in a variety of forms. These include, for example, liquid, semi-solid and solid dosage forms, such as liquid solutions (e.g., injectable and infusible solutions), dispersions or suspensions, tablets, capsules, pills, powders, liposomes, lipid nanoparticles and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g., intravenous, subcutaneous, intraperitoneal, intramuscular). In another embodiment, the compound is administered by intravenous infusion or injection. In yet another embodiment, the compound is administered by intramuscular or subcutaneous injection.

Oral administration of a solid dosage form may be, for example, presented in discrete units, such as hard or soft capsules, pills, cachets, lozenges, or tablets, each containing a predetermined amount of at least one compound of the invention. In another embodiment, the oral administration may be in a powder or granule form. In another embodiment, the oral dosage form is sub-lingual, such as, for example, a lozenge. In such solid dosage forms, the compounds of the invention are ordinarily combined with one or more adjuvants. Such capsules or tablets may comprise a controlled release formulation. In the case of capsules, tablets, and pills, the dosage forms also may comprise buffering agents or may be prepared with enteric coatings.

In another embodiment, oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as one or more of wetting, emulsifying, suspending, flavoring (e.g., sweetening), or perfuming agents.

In another embodiment, the invention comprises a parenteral dosage form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using one or more of suitable dispersing, wetting agents, or suspending agents.

In another embodiment, the invention comprises a topical dosage form. “Topical administration” includes, for example, dermal and transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated—see, for example, B. C. Finnin and T. M. Morgan, J. Pharm. Sci., vol. 88, pp. 955-958, 1999.

Formulations suitable for topical administration to the eye include, for example, eye drops wherein the compound of this invention is dissolved or suspended in a suitable excipient. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration, the compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the invention comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5) alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

Liposome containing compounds of the invention may be prepared by methods known in the art (See, for example, Chang, H. I.; Yeh, M. K.; Clinical development of liposome-based drugs: formulation, characterization, and therapeutic efficacy; Int J Nanomedicine 2012; 7; 49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Lipid nanoparticles (LNPs) comprising the compounds of the invention may be prepared by methods known in the art. Lipid nanoparticles (LNPs) constitute an alternative to other particulate systems, such as emulsions, liposomes, micelles, microparticles and/or polymeric nanoparticles, for the delivery of active ingredients, such as oligonucleotides, RNA and small molecule pharmaceuticals, and the adjuvant compounds of the invention. LNPs and their use for the delivery of oligonucleotides have been previously disclosed. See U.S. Pat. Nos. 7,691,405 and 11,406,706, U.S. patent application Publication Nos: US 2006/0083780, US 2006/0240554, US 2008/0020058, US 2009/0263407 and US 2009/0285881; and International Patent Application Publication Nos.: WO 2009/086558, WO2009/127060, WO2009/132131, WO2010/042877, WO2010/054384, WO2010/054401, WO2010/054405 and WO2010/054406. See also Semple et al., 2010, Nat. Biotechnol. 28:172-176. LNPs and their use for delivery of RNA vaccines have been previously disclosed. See International Patent Application Publication Nos.: WO2021213924 and WO2023019181. Lipid-based nanoparticles as pharmaceutical drug carriers have also been disclosed. See Puri et al., 2009, Crit. Rev. Ther. Drug Carrier Syst. 26:523-580. Cationic lipids are disclosed in U.S. Patent Application Publication Nos. US 2009/0263407, US 2009/0285881, US 2010/0055168, US 2010/0055169, US 2010/0063135, US 2010/0076055, US 2010/0099738 and US 2010/0104629, and U.S. Pat. No. 10,166,298. Lipid nanoparticle capsules are described in U.S. Patent Application Publication No. 2013/0017239. The compounds of the invention may be embedded in the lipid layer of the LNP for targeting of the LNP comprising an active ingredient (i.e. oligonucleotide, RNA, small molecule, etc) to the lymph nodes via the TLR7/8 moiety of the compounds of the invention.

Compounds of the invention may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, lipid nanoparticles, albumin microspheres, microemulsions, nanoparticles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing a compound of the invention, which matrices are in the form of shaped articles, e.g., films, or microcapsules. Examples of sustained-release matrices include polyesters, hydrogels (for example, poly(2-hydroxyethyl-methacrylate), or ′poly(vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the invention are generally placed into a container having a sterile access port, for example, an intravenous solution bag or vial having a stopper pierceable by a hypodermic injection needle.

Suitable emulsions may be prepared using commercially available fat emulsions, such as a lipid emulsions comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion may comprise fat droplets between 0.1 and 1.0 μm, particularly 0.1 and 0.5 μm, and have a pH in the range of 5.5 to 8.0.

For example, the emulsion compositions may be those prepared by mixing a compound of the invention with a lipid emulsions comprising soybean oil or the components thereof (soybean oil, egg phospholipids, glycerol and water).

Compositions for inhalation or insufflation include solutions and suspensions in pharmaceutically acceptable aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

A drug product intermediate (DPI) is a partly processed material that must undergo further processing steps before it becomes bulk drug product. Compounds of the invention may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid state stability and downstream manufacturability. In one embodiment, the drug product intermediate contains a compound of the invention isolated and stabilized in the amorphous state (for example, amorphous solid dispersions (ASDs)). There are many techniques known in the art to manufacture ASD's that produce material suitable for integration into a bulk drug product, for example, spray dried dispersions (SDD's), melt extrudates (often referred to as HME's), co-precipitates, amorphous drug nanoparticles, and nano-adsorbates. In one embodiment amorphous solid dispersions comprise a compound of the invention and a polymer excipient. Other excipients as well as concentrations of said excipients and the compound of the invention are well known in the art and are described in standard textbooks. See, for example, “Amorphous Solid Dispersions Theory and Practice” by Navnit Shah et al.

### Administration and Dosing

The term “treating”, “treat” or “treatment” as used herein embraces both preventative, i.e., prophylactic, and palliative treatment, i.e., relieve, alleviate, or slow the progression of the patient's disease (or condition) or any tissue damage associated with the disease.

As used herein, the terms, “subject, “individual” or “patient,” used interchangeably, refer to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting (or slowing) further development of the pathology or symptomatology or both); and
(3) ameliorating the disease; for example, ameliorating a disease, condition or disorder in an individual that is experiencing or displaying the pathology or symptomatology of the disease, condition or disorder (i.e., reversing the pathology or symptomatology or both).

Typically, a compound of the invention is administered in an amount effective to treat a condition as described herein. The compounds of the invention may be administered as compound per se, or alternatively, as a pharmaceutically acceptable salt. For administration and dosing purposes, the compound per se or pharmaceutically acceptable salt thereof will simply be referred to as the compounds of the invention.

The compounds of the invention are administered by any suitable route in the form of a pharmaceutical composition adapted to such a route, and in a dose effective for the treatment intended. The compounds of the invention may be administered orally, rectally, vaginally, parenterally, topically, intranasally, or by inhalation.

The compounds of the invention may be administered orally. Oral administration may involve swallowing, so that the compound enters the gastrointestinal tract, or buccal or sublingual administration may be employed by which the compound enters the bloodstream directly from the mouth.

In another embodiment, the compounds of the invention may also be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In another embodiment, the compounds of the invention may also be administered topically to the skin or mucosa, that is, dermally or transdermally. In another embodiment, the compounds of the invention may also be administered intranasally or by inhalation. In another embodiment, the compounds of the invention may be administered rectally or vaginally. In another embodiment, the compounds of the invention may also be administered directly to the eye or ear.

The dosage regimen for the compounds of the invention or compositions containing said compounds is based on a variety of factors, including the type, age, weight, sex and medical condition of the patient; the severity of the condition; the route of administration; and the activity of the particular compound employed. Thus, the dosage regimen may vary widely. In one embodiment, the total daily dose of a compound of the invention is typically from about 0.01 to about 100 mg/kg (i.e., mg compound of the invention per kg body weight) for the treatment of the indicated conditions discussed herein. In another embodiment, total daily dose of the compound of the invention is from about 0.1 to about 50 mg/kg, and in another embodiment, from about 0.5 to about 30 mg/kg. It is not uncommon that the administration of the compounds of the invention will be repeated a plurality of times in a day (typically no greater than 4 times). Multiple doses per day typically may be used to increase the total daily dose, if desired.

### Therapeutic Methods and Uses

The compounds of the invention may agonize or modulate the activity of TLR7 and/or TLR8 and may be useful as vaccine adjuvants.

Adjuvant formulations comprising the compounds of the invention may be used with an immunogen (i.e. a therapeutic agent or antigen of interest) to obtain an immunogenic composition, for example, a vaccine. The immunogenic composition may comprise naturally-occurring or artificially-created proteins, recombinant proteins, glycoproteins, peptides, carbohydrates, nucleic acids, haptens, whole viruses, bacteria, protozoa, or virus-like particles, or conjugates thereof as the immunogen. The immunogenic composition may be suitably used as a vaccine for chickenpox or shingles, human respiratory syncytial virus infection (RSV), Cytomegalovirus infection (CMV), Human metapneumovirus, Human parainfluenza viruses type 1 or type 3, Lyme disease, Streptococcus pneumonia, Clostridioides difficile, Escherichia coli or Klebsiella pneumoniae, influenza, HIV-1, Hepatitis A, Hepatitis B, Human Papilloma virus, Meningococcal type A meningitis, Meningococcal type B meningitis, Meningococcal type C meningitis, Tetanus, Diphtheria, Pertussis, Polio, Haemophilus influenza type B, Dengue, Hand Foot and Mouth Disease, Typhoid, Pneumococcus, Japanese encephalitis virus, Anthrax, Shingles, Malaria, Norovirus, or cancer. The immunogenic composition may be suitably used in methods for treating or preventing a disease or infection in a subject, preferably wherein the subject is a human, caused by a pathogen associated with an infectious disease wherein the pathogen is selected from Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli O157:H7, 0111 and 0104:H4, Escherichia coli Fimbrial antigen H, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV) including hMPV A and hMPV B, hMPV F protein, Human papillomavirus (HPV), Human parainfluenza viruses (HPIV), Japanese encephalitis virus, JC virus, Junin virus, Kingella kingae, Klebsiella granulomatis, Klebsiella pneumoniae, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parainfluenza virus (PIV) including PIV1, PIV2, PIV3, and PIV4, PIV1 F protein, PIV3 F protein, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella-zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

In some embodiments, adjuvant formulations comprising the compounds disclosed herein may be used with an RSV antigen to obtain an immunogenic composition. In some embodiments, the immunogenic composition comprising an adjuvant compound disclosed herein further comprises an RSV antigen comprising a RSV F protein. In some embodiments, the immunogenic composition comprising an adjuvant compound disclosed herein further comprises an RSV antigen comprising a RSV F protein of subtype A. In some embodiments, the immunogenic composition comprising an adjuvant compound disclosed herein further comprises an RSV antigen comprising a RSV F protein of subtype B. In some embodiments, the immunogenic composition comprising an adjuvant compound disclosed herein further comprises an RSV antigen comprising a mutant of a wild-type RSV F protein of subtype A. In some embodiments, the immunogenic composition comprising an adjuvant compound disclosed herein further comprises an RSV antigen comprising a nucleic acid encoding a mutant of a wild-type RSV F protein of subtype A, for example, a modRNA encoding a mutant of a wild-type RSV F protein of subtype A. In some embodiments, the RSV mutant is in the form of a trimer. In a particular embodiment, the RSV mutant is in the prefusion conformation. In another particular embodiment, the mutant is in the prefusion conformation and is in the form of a trimer.

In some embodiments, the RSV antigen is disclosed in one of WO2009/079796, WO2010/149745, WO2011/008974, WO2014/160463, WO2014/174018, WO2014/202570, WO2015/013551, WO2015/177312, WO2017/005848, WO2017/174564, WO2017/005844, WO2017/109629, WO2022/002894, or WO2018/109220. In one embodiment, the RSV antigen is a mutant of a wild-type RSV F protein of subtype A comprising the mutations T103C, I148C, S190I, and D486S. In another embodiment, the RSV antigen is a nucleic acid, for example modRNA, encoding a mutant of a wild-type RSV F protein of subtype A comprising the mutations T103C, I148C, S190I, and D486S.

In some embodiments, the immunogenic composition comprising an adjuvant compound disclosed herein further comprises an RSV antigen comprising a mutant of a wild-type RSV F protein of subtype B. In some embodiments, the immunogenic composition comprising an adjuvant compound disclosed herein further comprises an RSV antigen comprising a nucleic acid encoding a mutant of a wild-type RSV F protein of subtype B, for example, a modRNA encoding a mutant of a wild-type RSV F protein of subtype B. In some embodiments, the mutant is in the form of a trimer. In some embodiments, the mutant is in the prefusion conformation. In some embodiments, the mutant is in the prefusion conformation and is in the form of a trimer. In some embodiments, the RSV antigen is disclosed in one of WO2009/079796, WO2010/149745, WO2011/008974, WO2014/160463, WO2014/174018, WO2014/202570, WO2015/013551, WO2015/177312, WO2017/005848, WO2017/174564, WO2017/005844, WO2017/109629, WO2022/002894, or WO2018/109220. In one embodiment, the RSV antigen is a mutant of a wild-type RSV F protein of subtype B comprising the mutations T103C, I148C, S190I, and D486S. In another embodiment, the RSV antigen is a nucleic acid, for example modRNA, encoding a mutant of a wild-type RSV F protein of subtype B comprising the mutations T103C, I148C, S190I, and D486S.

In some embodiments, the immunogenic composition comprising an adjuvant compound disclosed herein further comprises an RSV antigen comprising a mutant of a wild-type RSV F protein of subtype A and an RSV antigen comprising a mutant of a wild-type RSV F protein of subtype B. In some embodiments, the immunogenic composition comprising an adjuvant compound disclosed herein further comprises an RSV antigen comprising a nucleic acid encoding a mutant of a wild-type RSV F protein of subtype A, for example, a modRNA encoding a mutant of a wild-type RSV F protein of subtype A and an RSV antigen comprising a nucleic acid encoding a mutant of a wild-type RSV F protein of subtype B, for example, a modRNA encoding a mutant of a wild-type RSV F protein of subtype B. In some embodiments, the mutant of subtype A or B is in the form of a trimer. In some embodiments, the mutant of subtype A or B is in the prefusion conformation. In some embodiments, the mutant of subtype A or B is in the prefusion conformation and is in the form of a trimer. In some embodiments, the RSV antigen of subtype A or B is disclosed in one of WO2009/079796, WO2010/149745, WO2011/008974, WO2014/160463, WO2014/174018, WO2014/202570, WO2015/013551, WO2015/177312, WO2017/005848, WO2017/174564, WO2017/005844, WO2017/109629, WO2022/002894, or WO2018/109220. In one embodiment, the immunogenic composition comprises an RSV antigen that is a mutant of a wild-type RSV F protein of subtype A comprising the mutations T103C, I148C, S190I, and D486S and an RSV antigen that is a mutant of a wild-type RSV F protein of subtype B comprising the mutations T103C, I148C, S190I, and D486S. In another embodiment, the immunogenic composition comprises an RSV antigen that is a nucleic acid, for example modRNA, encoding a mutant of a wild-type RSV F protein of subtype A comprising the mutations T103C, I148C, S190I, and D486S and a nucleic acid, for example modRNA, encoding a mutant of a wild-type RSV F protein of subtype B comprising the mutations T103C, I148C, S190I, and D486S.

In some embodiments, the immunogenic composition comprising an adjuvant compound disclosed herein further comprises a RSV F Protein of subtype A comprising the sequence of SEQ ID NO: 1. In some embodiments, the immunogenic composition comprising an adjuvant compound disclosed herein further comprises a RSV F Protein of subtype A comprising the sequence of SEQ ID NO: 2. In particular embodiments, the immunogenic composition comprising an adjuvant compound disclosed herein further comprises a RSV F Protein of subtype A comprising the sequence of SEQ ID NO: 1 and SEQ ID NO: 2.

In some embodiments, the immunogenic composition comprising an adjuvant compound disclosed herein further comprises a RSV F Protein of subtype B comprising the sequence of SEQ ID NO: 3. In some embodiments, the immunogenic composition comprising an adjuvant compound disclosed herein further comprises a RSV F Protein of subtype B comprising the sequence of SEQ ID NO: 4. In particular embodiments, the immunogenic composition comprising an adjuvant compound disclosed herein further comprises a RSV F Protein of subtype B comprising the sequence of SEQ ID NO: 3 and SEQ ID NO: 4.

In further particular embodiments, the immunogenic composition comprising an adjuvant compound disclosed herein further comprises a RSV F Protein of subtype A comprising the sequence of SEQ ID NO: 1 and SEQ ID NO: 2; and a RSV F Protein of subtype B comprising the sequence of SEQ ID NO: 3 and SEQ ID NO: 4.

RSV F Protein Subtype A, F2 amino acid
sequence (Ontario Strain), Residues
26-109 (T103C Mutant)
SEQ ID NO: 1:
QNITEEFYQSTCSAVSKGYLSALRTGWYTSVITIELSNIKENKCNGTDAK
VKLIKQELDKYKNAVTELQLLMQSTPACNSRARR
RSV F Protein Subtype A, F1 amino acid
sequence (Ontario Strain) Residues
137-513 (1148C, S1901, and D486S
Mutants)
SEQ ID NO: 2:
FLGFLLGVGSACASGIAVSKVLHLEGEVNKIKSALLSTNKAVVSLSNGVS
VLTIKVLDLKNYIDKQLLPIVNKQSCSISNIETVIEFQQKNNRLLEITRE
FSVNAGVTTPVSTYMLTNSELLSLINDMPITNDQKKLMSSNVQIVRQQSY
SIMSIIKEEVLAYVVQLPLYGVIDTPCWKLHTSPLCTTNTKEGSNICLTR
TDRGWYCDNAGSVSFFPQAETCKVQSNRVFCDTMNSLTLPSEVNLCNIDI
FNPKYDCKIMTSKTDVSSSVITSLGAIVSCYGKTKCTASNKNRGIIKTFS
NGCDYVSNKGVDTVSVGNTLYYVNKQEGKSLYVKGEPIINFYDPLVFPSS
EFDASISQVNEKINQSLAFIRKSDELL
RSV F Protein Subtype B, F2 amino acid
sequence (Buenos Aires Strain), Residues
26-109 (T103C Mutant)
SEQ ID NO: 3:
QNITEEFYQSTCSAVSRGYFSALRTGWYTSVITIELSNIKETKCNGTDTK
VKLIKQELDKYKNAVTELQLLMQNTPACNNRARR
RSV F Protein Subtype B, F1 amino acid
sequence (Buenos Aires Strain) Residues
137-513 (1148C, S1901, and D486S
Mutants)
SEQ ID NO: 4:
FLGFLLGVGSACASGIAVSKVLHLEGEVNKIKNALLSTNKAVVSLSNGVS
VLTIKVLDLKNYINNQLLPIVNQQSCRISNIETVIEFQQKNSRLLEITRE
FSVNAGVTTPLSTYMLTNSELLSLINDMPITNDQKKLMSSNVQIVRQQSY
SIMSIIKEEVLAYVVQLPIYGVIDTPCWKLHTSPLCTTNIKEGSNICLTR
TDRGWYCDNAGSVSFFPQADTCKVQSNRVFCDTMNSLTLPSEVSLCNTDI
FNSKYDCKIMTSKTDISSSVITSLGAIVSCYGKTKCTASNKNRGIIKTFS
NGCDYVSNKGVDTVSVGNTLYYVNKLEGKNLYVKGEPIINYYDPLVFPSS
EFDASISQVNEKINQSLAFIRRSDELL

In some embodiments, adjuvant formulations comprising the compounds disclosed herein may be used with a Streptococcus pneumoniae antigen to obtain an immunogenic composition. In other embodiments, adjuvant formulations comprising the compounds disclosed herein may be used with a Streptococcus pneumoniae polysaccharide antigen to obtain an immunogenic composition. In other embodiments, adjuvant formulations comprising the compounds disclosed herein may be used with a Streptococcus pneumoniae serotype 3 polysaccharide antigen to obtain an immunogenic composition. In some embodiments, adjuvant formulations comprising the compounds disclosed herein may be used with 1, 2, 3, 4, 5, 6, 7, 8, 9, 10, 11, 12, 13, 14, 15, 16, 17, 18, 19, 20, 21, 22, 23, 24, 25, or more Streptococcus pneumoniae polysaccharide antigens.

In some embodiments, adjuvant formulations comprising the compounds disclosed herein may be used with a conjugated Streptococcus pneumoniae antigen to obtain an immunogenic composition. In other embodiments, adjuvant formulations comprising the compounds disclosed herein may be used with a conjugated Streptococcus pneumoniae polysaccharide antigen to obtain an immunogenic composition. In other embodiments, adjuvant formulations comprising the compounds disclosed herein may be used with a conjugated Streptococcus pneumoniae serotype 3 polysaccharide antigen to obtain an immunogenic composition. In some embodiments, adjuvant formulations comprising the compounds disclosed herein may be used with 1, 2, 3, 4, 5, 6, 7, 8, 9, 10, 11, 12, 13, 14, 15, 16, 17, 18, 19, 20, 21, 22, 23, 24, 25, or more conjugated Streptococcus pneumoniae polysaccharide antigens. In some embodiments, the Streptococcus pneumoniae polysaccharide antigens are conjugated to a CRM₁₉₇ carrier. In some embodiments, the Streptococcus pneumoniae polysaccharide antigens are conjugated to a C5a peptidase from Streptococcus (SCP) carrier. In a particular embodiment, the immunogenic composition comprises an adjuvant compound disclosed herein and a Streptococcus pneumoniae serotype 3 polysaccharide antigen conjugated to a CRM₁₉₇ carrier. In another particular embodiment, the immunogenic composition comprises an adjuvant compound disclosed herein and a Streptococcus pneumoniae serotype 3 polysaccharide antigen conjugated to a SCP carrier.

The present invention provides an immunogenic composition comprising an immunogen and a compound of the invention as described herein.

### Co-Administration

The compounds of the invention may be used alone, or in combination with one or more therapeutic agents. The invention provides any of the uses, methods or compositions as defined herein wherein the compound of the invention, or pharmaceutically acceptable salt thereof, is used in combination with one or more therapeutic agent discussed herein.

The administration of two or more compounds “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compounds at the same point in time but as separate dosage forms at the same or different site of administration. Examples of “in combination” include, but are not limited to, “concurrent administration,” “co-administration,” “simultaneous administration,” “sequential administration” and “administered simultaneously”.

A compound of the invention and the one or more therapeutic agents may be administered as a fixed or non-fixed combination of the active ingredients. The term “fixed combination” means a compound of the invention, or a pharmaceutically acceptable salt thereof, and the one or more therapeutic agents, are both administered to a subject simultaneously in a single composition or dosage. The term “non-fixed combination” means that a compound of the invention, or a pharmaceutically acceptable salt thereof, and the one or more therapeutic agents are formulated as separate compositions or dosages such that they may be administered to a subject in need thereof simultaneously or at different times with variable intervening time limits, wherein such administration provides effective levels of the two or more compounds in the body of the subject.

These agents and compounds of the invention may be combined with pharmaceutically acceptable vehicles such as saline, Ringer's solution, dextrose solution, and the like. The particular dosage regimen, i.e., dose, timing and repetition, will depend on the particular individual and that individual's medical history.

### Kits

Another aspect of the invention provides kits comprising the compound of the invention or pharmaceutical compositions comprising the compound of the invention. A kit may include, in addition to the compound of the invention or pharmaceutical composition thereof, diagnostic or therapeutic agents. A kit may also include instructions for use in a diagnostic or therapeutic method. In some embodiments, the kit includes the compound or a pharmaceutical composition thereof and a diagnostic agent. In other embodiments, the kit includes the compound or a pharmaceutical composition thereof and one or more therapeutic agents.

In yet another embodiment, the invention comprises kits that are suitable for use in performing the methods described herein. In one embodiment, the kit contains a first dosage form comprising one or more of the compounds of the invention in quantities sufficient to carry out the methods of the invention. In another embodiment, the kit comprises one or more compounds of the invention in quantities sufficient to carry out the methods of the invention and a container for the dosage.

### Synthetic Methods

Compounds of the present invention may be synthesized by synthetic routes that include processes analogous to those well-known in the chemical arts, particularly in light of the description contained herein. The starting materials are generally available from commercial sources or may be prepared using methods well known to those skilled in the art. Many of the compounds used herein, are related to, or may be derived from compounds in which one or more of the scientific interest or commercial need has occurred. Accordingly, such compounds may be one or more of 1) commercially available; 2) reported in the literature or 3) prepared from other commonly available substances by one skilled in the art using materials which have been reported in the literature.

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are discussed below, other starting materials and reagents may be substituted to provide one or more of a variety of derivatives or reaction conditions. In addition, many of the compounds prepared by the methods described below may be further modified in light of this disclosure using conventional chemistry well known to those skilled in the art.

The skilled person will appreciate that the experimental conditions set forth in the schemes that follow are illustrative of suitable conditions for effecting the transformations shown, and that it may be necessary or desirable to vary the precise conditions employed for the preparation of compounds of the invention. It will be further appreciated that it may be necessary or desirable to carry out the transformations in a different order from that described in the schemes, or to modify one or more of the transformations, to provide the desired compound of the invention.

In the preparation of compounds of the invention it is noted that some of the preparation methods useful for the preparation of the compounds described herein may require protection of remote functionality (e.g., a primary amine, secondary amine, carboxyl, etc. in a precursor of a compound of the invention). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see March's Advanced Organic Chemistry: Reactions, Mechanisms, and Structure 8th Edition.

For example, if a compound contains an amine or carboxylic acid functionality, such functionality may interfere with reactions at other sites of the molecule if left unprotected. Accordingly, such functionalities may be protected by an appropriate protecting group (PG) which may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines and lower alkyl or benzyl esters for carboxylic acids) which are generally not chemically reactive under the reaction conditions described and may typically be removed without chemically altering other functionality in a compound of the invention.

### General Experimental Details

In the non-limiting Examples and Preparations that illustrate the invention and that are set out in the description, and in the following Schemes, the following the abbreviations, definitions and analytical procedures may be referred to:

¹H Nuclear magnetic resonance (NMR) spectra were recorded on a 400 MHz Bruker and in all cases consistent with the proposed structures. Characteristic chemical shifts (δ) are given in parts-per-million referenced to residual peaks from the deuterated solvents employed (for ¹H-NMR) using conventional abbreviations for designation of major peaks: e.g. s, singlet; d, doublet; t, triplet; q, quartet; m, multiplet; br, broad; p, pentet; sept, septet; dd, doublet of doublets; dt, doublet of triplets; tt, triplet of triplets. The following abbreviations have been used for common solvents: CDCl₃, deuterochloroform; DMSO-d₆, deuterodimethylsulfoxide; and CD₃OD, deuteromethanol. All observed coupling constants, J, are reported in Hertz (Hz). Where appropriate, tautomers may be recorded within the NMR data; and some exchangeable protons are not always observed.

Mass spectra, MS (m/z), were recorded using either electrospray ionization (ESI) or atmospheric pressure chemical ionization (APCI). Where relevant and unless otherwise stated, the m/z data provided are for isotopes ¹⁹F, ³⁵Cl, ⁷⁹Br and ¹²⁷I.

Liquid chromatography/mass-spec (LCMS) purity and characterization methods used for the compounds set forth in the Examples include:

### i) Method A:

Acq Method: D:\method\5-95AB_1.5min_220&254_1500_Shimadzu.lcm
MS Ionization: ESI
Instrument & Column: LCMS-AO(4-220) Chromolith. Flash RP-18e 25-3 mm; and

### ii) Method B:

Acq Method: D:\method\5-95AB_1.5min_220&254_1500_Shimadzu.lcm
MS Ionization: ESI
Instrument & Column: LCMS-AH(4-220) Pursult 5 C18 20*2.0 mm,S/N 508907.

### Abbreviations

°2q is degrees 2-theta;
AcCl is acetyl chloride;
AcOH is acetic acid;
ADH-101 is alcohol dehydrogenase 101;
APCI is atmospheric pressure chemical ionization;
Atm is atmospheric pressure;
aq is aqueous;
BH₃Me₂S is (dimethyl sulphide)trihydroboron;
BINAP is 1,1′-binaphthalene-2,2′-diyl)bis(diphenylphosphine;
Bn is benzyl;
Boc is tert-butoxycarbonyl;
Boc₂O is di-tert-butyl dicarbonate;
br is broad;
tBu is tert-butyl;
tBuOH is tert-butanol;
tBuOK is potassium tert-butoxide;
tBuXPhos is 2-di-tert-butylphosphino-2′,4′,6′-triisopropylbiphenyl;
tBuXPhos-Pd Gen-3 is [(2-di-tert-butylphosphino-2′,4′,6′-triisopropyl-1,1′-biphenyl)-2-(2′-amino-1,1′-biphenyl)] palladium(II) methanesulfonate;
° C. is degrees celcius;
CDCl₃ is deutero-chloroform;
CDI is 1,1′-carbonyldiimidazole;
Cs₂CO₃ is cesium carbonate;
CuI is copper(I) iodide;
δ is chemical shift;
d is doublet;
dd is doublet of doublets;
ddd is doublet of doublet of doublets;
dt is doublet of triplets;
DCE is 1,2-dichloroethane;
DCM is dichloromethane; methylene chloride;
DIAD is diisopropyl azodicarboxylate;
(−)-DIP-Chloride™ is (−)-B-chlorodiisopinocampheylborane;
DIPEA is N-ethyldiisopropylamine, also known as N,N-diisopropylethylamine;
DMA is N,N-dimethylacetamide;
DME is 1,2-dimethoxyethane;
DMAP is 4-dimethylaminopyridine;
DMF is N,N-dimethylformamide;
DMSO is dimethyl sulfoxide;
DMSO-d₆ is deuterodimethylsulfoxide;
DPPP is 1,3-bis(diphenylphosphino)propane;
EDC is N-(3-dimethylaminopropyl)-N′-ethylcarbodiimide;
EDC·HCl is N-(3-dimethylaminopropyl)-N′-ethylcarbodiimide hydrochloride;
ESI is electrospray ionization;
Et₂O is diethyl ether;
EtOAc is ethyl acetate;
EtOH is ethanol;
Et₃N is triethylamine;
g is gram;
HATU is 1-[bis(dimethylamino)methylene]-1H-1,2,3-triazolo[4,5-b]pyridinium 3-oxid hexafluorophosphate;
HCl is hydrochloric acid;
HPLC is high pressure liquid chromatography;
HOBt is 1-hydroxybenzotriazole hydrate;
h(s) is hour(s);
IPA is isopropyl alcohol;
iPrOAc is isopropyl acetate;
Ir[dF(CF₃)ppy]₂(dtbpy)PF₆ is [4,4′-bis(1,1-dimethylethyl)-2,2′-bipyridine-N1,N1′]bis[3,5-difluoro-2-[5-(trifluoromethyl)-2-pyridinyl-N]phenyl-C]iridium(III) hexafluorophosphate;
K₂CO₃ is potassium carbonate;
KRED101 is ketoreductase 101 enzyme;
L is liter;
LCMS is liquid chromatography mass spectrometry;
m is multiplet;
M is molar;
m-CPBA is 3-chloroperbenzoic acid;
MeCN is acetonitrile;
MeMgBr is methylmagnesium bromide;
MeNHOMe HCl is N,O-dimethylhydroxylamine hydrochloride;
MeOD-d₄ is deuterated methanol;
MeOH is methanol;
2-MeTHF is 2-methyl tetrahydrofuran;
mg is milligram;
MHz is mega Hertz;
min(s) is minute(s);
mL is milliliter;
mmol is millimole;
mol is mole;
MS (m/z) is mass spectrum peak;
MsCl is mesyl chloride;
MTBE is tert-butyl methyl ether;
NADP+ is nicotinamide adenine dinucleotide phosphate;
NH₄HCO₃ is ammonium bicarbonate;
NiCl₂·glyme is nickel (II) chloride ethylene glycol dimethyl ether complex;
NMR is nuclear magnetic resonance;
ODS is octadecyl-silica;
ORTEP is Oak Ridge Thermal Ellipsoid Plot;
Pd(tBu₃P)₂ is bis(tri-tert-butylphosphine)palladium(0);
Pd/C is palladium on carbon;
Pd₂(dba)₃ is palladium tris(dibenzylideneacetone)dipalladium(0);
Pd(dppf)Cl₂ is [1,1′-bis(diphenylphophino)ferrocene]dichloropalladium(II);
Pd(PPh₃)₄ is tetrakis(triphenylphosphine)palladium(0);
Pet. ether is the petroleum fraction consisting of aliphatic hydrocarbons and boiling in the range 35-60° C.;
PMB is para-methoxybenzyl;
PMB-NH₂ is para-methoxybenzylamine;
Polycat 5 ® is bis(2-dimethylaminoethyl)(methyl)amine
PPh₃ is triphenylphosphine;
pH is power of hydrogen;
ppm is parts per million;
PSD is position sensitive detector;
psi is pounds per square inch;
Pt/C is platinum on carbon;
PXRD is powder X-ray diffraction;
q is quartet;
rt is room temperature;
RT is retention time;
s is singlet;
SEM-Cl is 2-(trimethylsilyl)ethoxymethyl chloride;
SFC is supercritical fluid chromatography;
t is triplet;
T₃P is propylphosphonic anhydride;
TBAF is tert-butyl ammonium fluoride;
TBAI is tetrabutylammonium iodide;
TBDMSCl is tert-butyldimethylsilyl chloride;
TFA is trifluoroacetic acid;
THF is tetrahydrofuran;
TLC is thin layer chromatography;
TMEDA is N,N,N′N′-tetramethylethylenediamine;
TMSCl is trimethylsilyl chloride;
TMSCN is trimethylsilyl cyanide;
TMSCHN₂ is (diazomethyl)trimethylsilane;
TsCl is p-toluenesulfonyl chloride;
Ts₂O is p-toluenesulfonic anhydride;
μL is microliter;
μmol is micromole; and
Xantphos is 4,5-bis(diphenylphosphno)-9,9-dimethylxanthene

The Schemes described below are intended to provide a general description of the methodology employed in the preparation of the compounds of the present invention. Some of the compounds of the present invention contain a single chiral center. In the following Schemes, the general methods for the preparation of the compounds are shown either in racemic or enantioenriched form. It will be apparent to one skilled in the art that all of the synthetic transformations may be conducted in a precisely similar manner whether the materials are enantioenriched or racemic. Moreover, the resolution to the desired optically active material may take place at any desired point in the sequence using well known methods such as described herein and in the chemistry literature.

Compounds of the invention may be made according to the following Schemes I-V, although alternative methodologies may also be utilized. One skilled in the art will appreciate that alternative reaction conditions to the ones illustrated in the schemes and examples may be utilized as deemed appropriate. Choices of solvents, additives such as acidic or basic catalysts, coupling agents, and indeed the reaction sequence may be changed as appropriate for a given target compound.

### General Methods:

Unless stated otherwise, the variables in Schemes I-V have the same meanings as defined herein.

In some cases, compounds of Formula (I), (Ia), (Ia(i)), (Ia(ii)), (Ia(iii)), (Ia(iv)), (Ib), (Ib(i)), (Ib(ii)), (Ib(iii)) or (Ib(iv)) may contain protecting groups, which may be appended or removed by additional steps in the synthetic sequence using conditions known in the art (March's Advanced Organic Chemistry: Reactions, Mechanisms, and Structure 8th Edition or Protecting Groups, 10 Georg Thieme Verlag, 1994). Compounds at every step may be purified by standard techniques, such as column chromatography, crystallization, or reverse phase SFC or HPLC. Variables A, L, R, X, Y, Z, m, n and p are as defined in the embodiments, schemes, examples, and claims herein.

Examples of the current invention whereby the albumin binding moiety is cholesterol or a derivative thereof may be made according to Scheme I-IV.

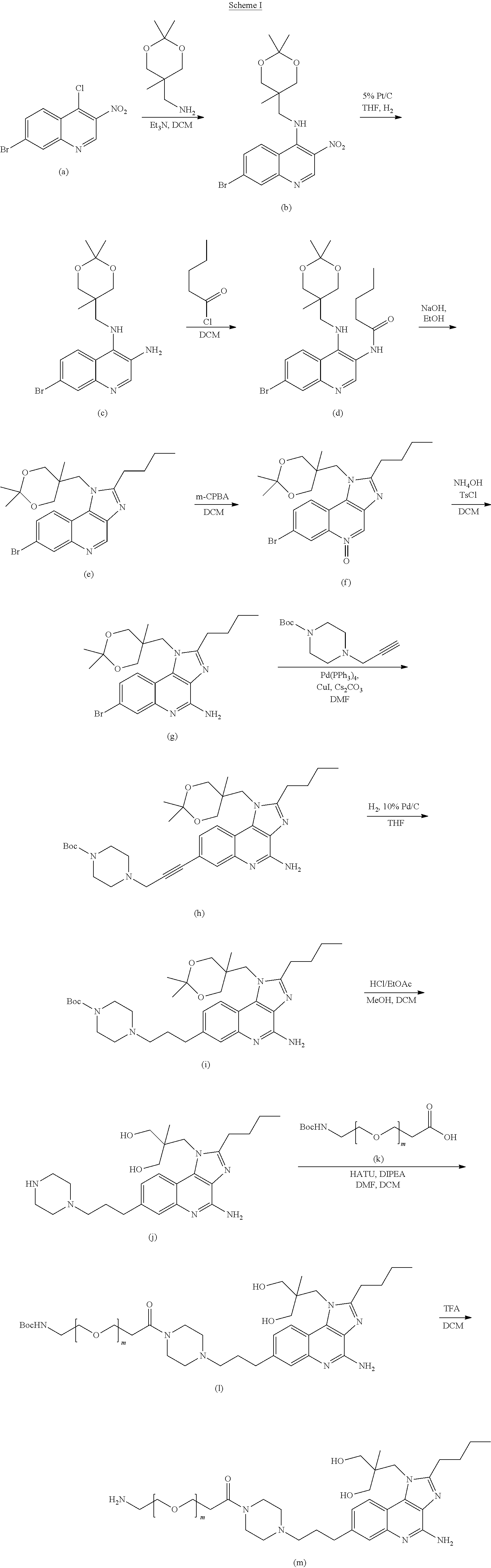

The chloride (a) may be reacted with (2,2,5-trimethyl-1,3-dioxan-5-yl)methanamine in a suitable solvent such as dichloromethane with a base such as triethylamine to afford the arylamine (b). The nitro aromatic intermediate (b) may then be reduced by for example catalytic hydrogenation over a suitable catalyst such as platinum in a suitable solvent (eg tetrahydrofuran or ethanol) to afford the aniline derivative (c). Alternative reducing conditions may also be applied such as a dissolving metal (iron in acetic acid). The aniline (c) may then be acylated (acid chloride, acid anhydride, carboxylic acid and coupling agent) to afford the amide (d), which can be subsequently cyclized under basic conditions (NaOH in ethanol) to afford the tricycle (e). Oxidation with metachloroperoxybenzoic acid affords intermediate (f) which may be treated with ammonium hydroxide and an activating agent such as p-toluene sulfonyl chloride to afford the amine (g). Coupling of compound (g) with tert-butyl 4-(prop-2-yn-1-yl)piperazine-1-carboxylate under transition metal catalysis forms the acetylene intermediate (h), which can be reduced with catalytic hydrogenation over a catalyst, such as palladium on carbon in a solvent like tetrahydrofuran, to afford the reduced intermediate (i). Removal of the protecting group under standard acidic conditions (like hydrochloric acid in ethyl acetate, methanol and dichloromethane) gives the amine (j) which may then be reacted with the selected protected PEG amino acid (k) under suitable coupling conditions, for example HATU and Hunig's base (N,N-diisopropylethylamine) in a suitable solvent, to give the protected intermediate (l). Deprotection of the terminal amine under standard acidic conditions (such as trifluoroacetic acid in dichloromethane) can then afford the desired compound (m).

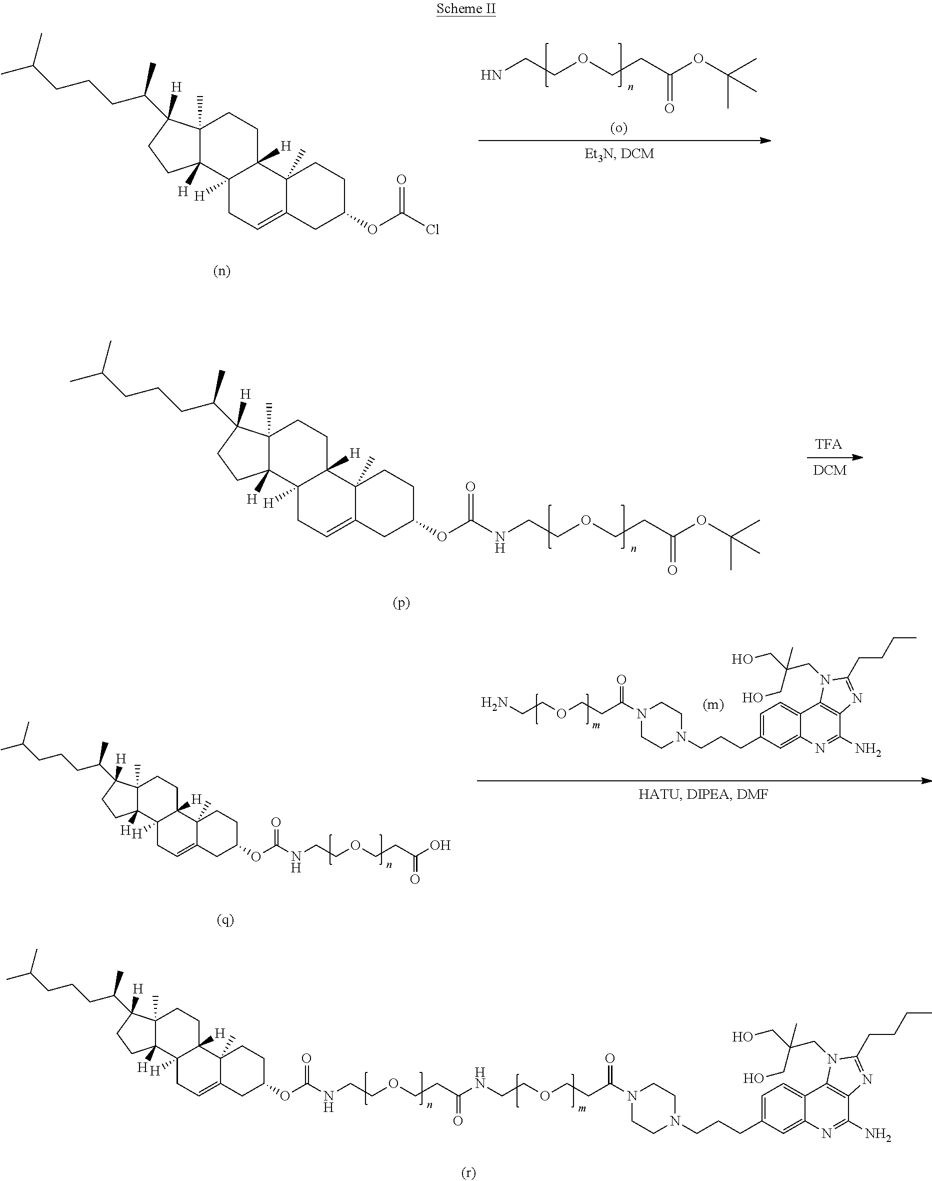

Cholesterol chloroformate (n) may be reacted with the selected protected PEG amino acid (o) with a base such as triethylamine and in a solvent like dichloromethane to give the carbamate (p) which is deprotected under standard acidic conditions (such as trifluoroacetic acid in dichloromethane) to afford the carboxylic acid (q). Subsequent reaction of the acids (q) with the amines (in) under appropriate coupling conditions, for example (HATU and Hunig's base (N,N-diisopropylethylamine) in dimethyl formamide) afford the desired products (r).

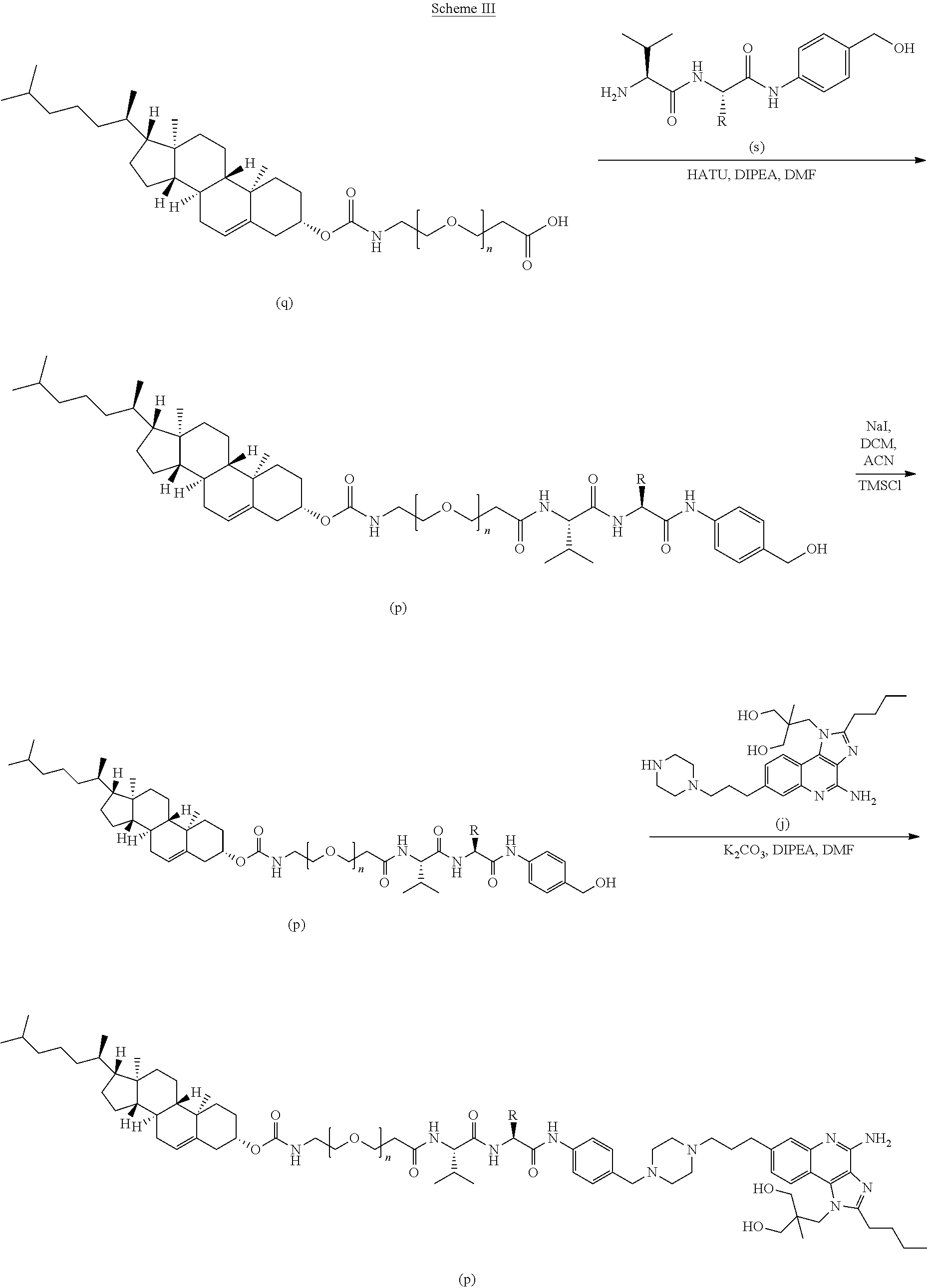

The acid (q) can react with the amine intermediate (s) under standard coupling conditions, like HATU with N,N-diisopropylethylamine in dimethyl formamide to form the benzyl alcohol intermediate (t). The benzyl alcohol intermediate (t) in solvents like dichloromethane and acetonitrile can be reacted sequentially with sodium iodide then trimethylsilyl chloride in dichloromethane to provide the iodo-intermediate (u). By reacting the iodo-intermediate (u) with the amine j) under standard SNAr conditions, like potassium carbonate with N,N-diisopropylethylamine in dimethyl formamide, can give the desired products (v).

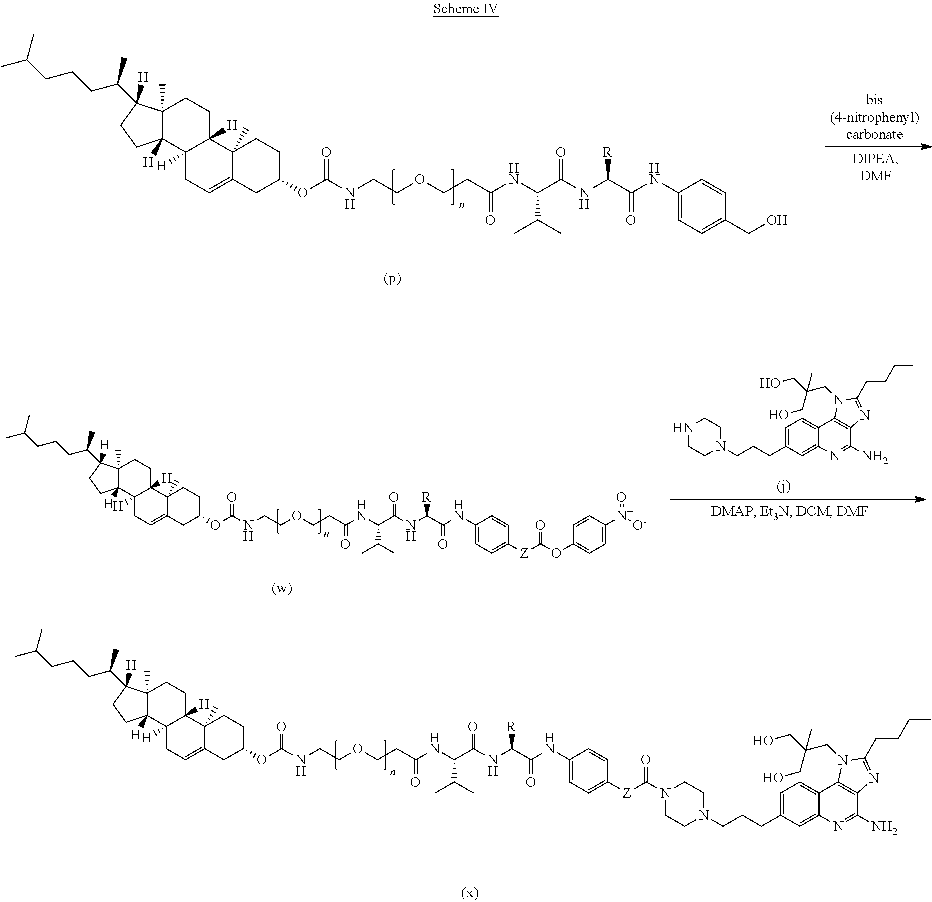

The benzyl alcohol intermediate (t) may be reacted with bis(4-nitrophenyl) carbonate under standard SNAr conditions, like N,N-diisopropylethylamine in dimethyl formamide, to form the nitro-intermediate (w). The nitro-intermediate (w) can then react with the amine j) under standard SNAr conditions, such as with triethylamine in dichloromethane and dimethyl formamide, catalyzed by 4-dimethylaminopyridine to give the desired products (x).

Examples of the current invention whereby the albumin binding moiety is tocopherol or a derivative thereof may be made according to Scheme V.

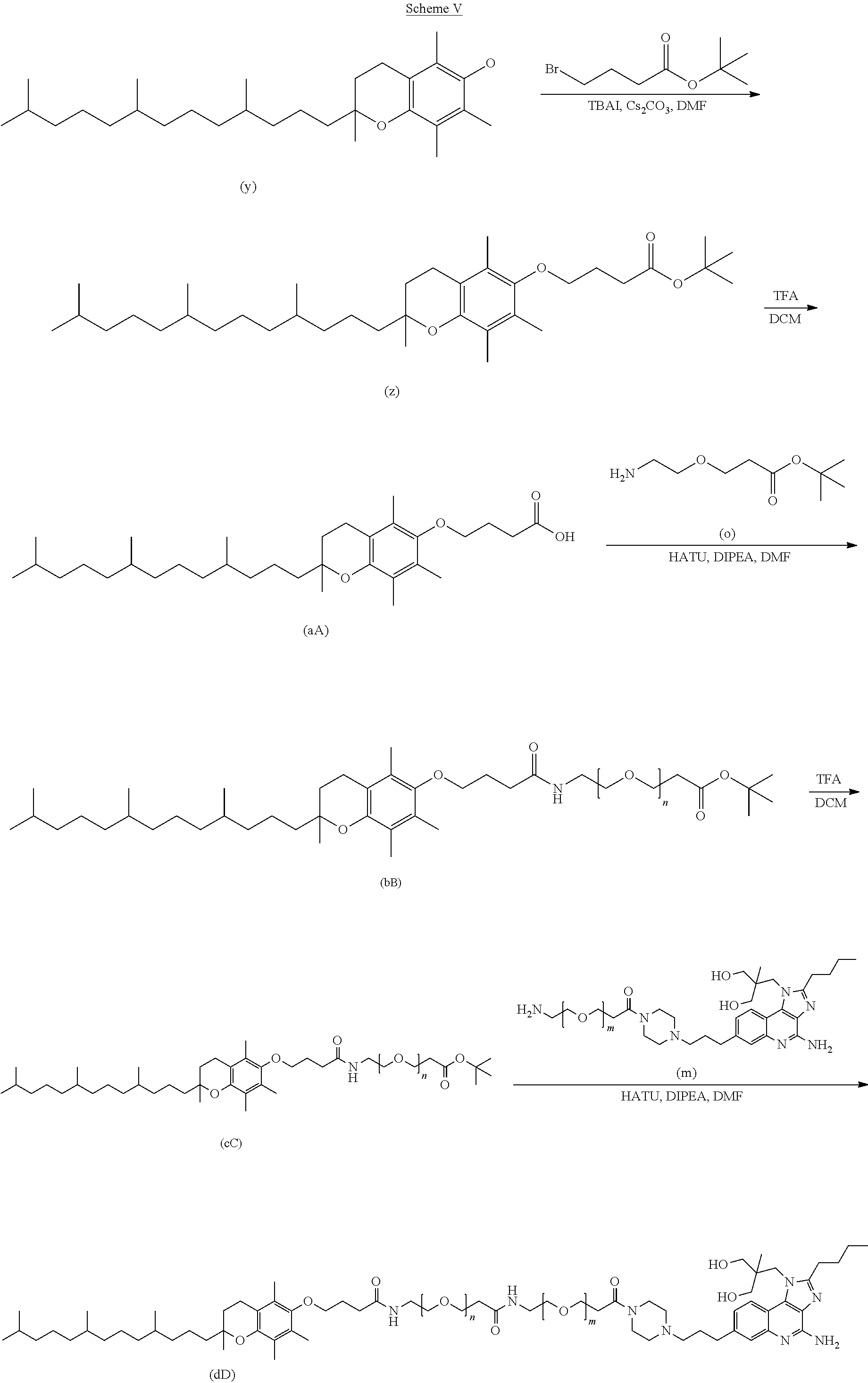

Tocopherol (y) is reacted under basic conditions with tert-butyl 4-bromobutanoate to afford the ether (z) which is subsequently deprotected under standard acidic conditions, such as trifluoroacetic acid in dichloromethane, to give the acid (aA). The acid (aA) can react with the protected PEG amino acid (o) through standard amide couple conditions, like HATU and N,N-diisopropylethylamine in dimethyl formamide, to afford the amide intermediate (bB). Deprotection of the amide intermediate (bB) can occur through acidic conditions (such as trifluoroacetic acid in dichloromethane) to provide the acid (cC). The acid (cC) can then react with the amines (m) with a coupling agent such as HATU in the presence of a base (like Hunig's base (N,N-diisopropylethylamine) to afford the target tocopherol derivatives (dD).

The synthetic intermediates having formulae a, b, c, d, e, f, g, h, i, j, k, l, m, n, o, p, q, r, s, t, u, v, w, x, y, z, aA, bB, cC and dD as defined in the above Schemes are useful for preparing compounds of the invention and are provided as further aspects of this invention.

In executing the synthesis of the compounds of the invention, one skilled in the art will monitor reactions with common methods that include thin-layer chromatography (TLC), liquid chromatography/mass spectroscopy (LCMS), and nuclear magnetic resonance (NMR).

One skilled in the art will also recognize that the compounds of the invention may be prepared as mixtures of diastereomers or geometric isomers (e.g., cis and trans substitution on a cycloalkane ring). These isomers can be separated by standard chromatographic techniques, such as normal phase chromatography on silica gel, reverse phase preparative high pressure liquid chromatography or supercritical fluid chromatography. One skilled in the art will also recognize that some compounds of the invention are chiral and thus may be prepared as racemic or scalemic mixtures of enantiomers. Several methods are available and are well known to those skilled in the art for the separation of enantiomers.

### EXAMPLES

In order that this invention may be better understood, the following examples are set forth. These examples are for purposes of illustration only and are not to be construed as limiting the scope of the invention in any manner.

The compounds and intermediates described below were named using the naming convention provided with ACD Labs Version 12 (ACD Labs, Toronto, Ontario, Canada). The naming convention provided with ACD Labs Version 12 is well known by those skilled in the art and it is believed that the naming convention provided with ACD Labs Version 12 generally comports with the IUPAC (International Union for Pure and Applied Chemistry) recommendations on Nomenclature of Organic Chemistry and the CAS Index rules. Unless noted otherwise, all reactants were obtained commercially without further purifications or were prepared using methods known in the literature.

### Example 1

(3beta)-cholest-5-en-3-yl {91-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-15,91-dioxo-3,6,9,12,19,22,25,28,31,34,37,40,43,46,49,52,55,58,61,64,67,70,73,76,79,82,85,88-octacosaoxa-16-azahennonacont-1-yl}carbamate (Compound 1)

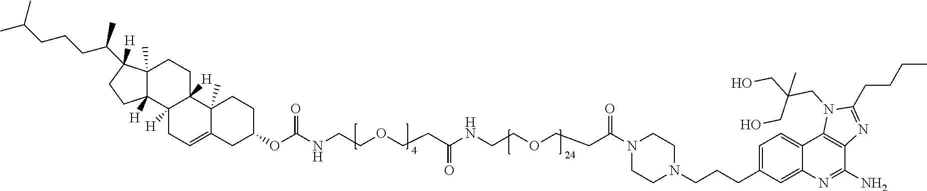

### Step (i): tert-butyl 1-[(3p)-cholest-5-en-3-yloxy]-1-oxo-5,8,11,14-tetraoxa-2-azaheptadecan-17-oate

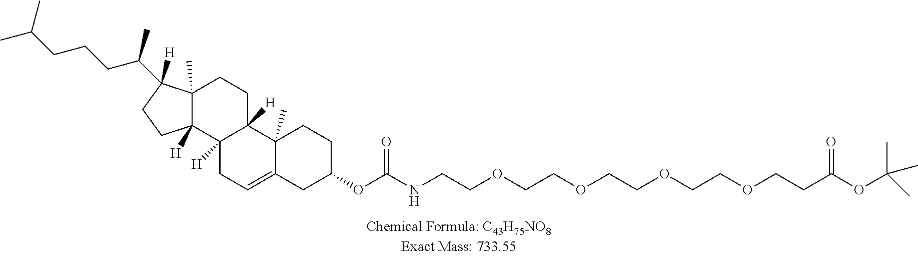 [SMILES: CC(C)CCC[C@@H](C)[C@H]1CC[C@H]2[C@@H]3CC=C4C[C@@H](OC(=O)NCCOCCOCCOCCOCCC(=O)OC(C)(C)C)CC[C@]4(C)[C@H]3CC[C@]12C]

A solution of cholesteryl chloroformate (CAS: 7144-08-3, 5 g, 11.1 mmol) in DCM was treated with triethylamine (Et₃N) (1.69 g, 16.7 mmol) at 0° C. under a nitrogen atmosphere. The reaction mixture was then treated with tert-butyl 1-amino-3,6,9,12-tetraoxapentadecan-15-oate (CAS: 581065-95-4, 3.58 g, 11.1 mmol) dropwise at 0° C. The reaction mixture was allowed to warm to rt and stirred for 16 h. The reaction mixture was diluted with DCM (50 mL), washed with brine (50 mL), dried over sodium sulfate, filtered, and concentrated in vacuo to give a colorless residue, which was purified by column chromatography (silica, DCM/MeOH, 100:0 to 9:1) to give the title compound (7.33 g, 89%) as a colorless oil.

¹H NMR (400 MHz, CDCl₃) δ 5.38 (s, 1H), 5.30 (br s, 1H), 4.50 (br s, 1H), 3.73 (t, 2H), 3.61-3.70 (m, 12H), 3.57 (t, 2H), 3.35-3.40 (m, 2H), 2.53 (t, 2H), 2.24-2.42 (m, 2H), 1.93-2.07 (m, 2H), 1.81-1.93 (m, 3H), 1.49-1.64 (m, 6H), 1.46 (s, 9H), 1.23-1.39 (m, 5H), 1.07-1.19 (m, 7H), 0.91-1.04 (m, 5H), 0.84-0.90 (m, 1H), 0.85-0.90 (m, 6H), 0.69 (s, 3H).

### Step (ii): 1-[(3p)-cholest-5-en-3-yloxy]-1-oxo-5,8,11,14-tetraoxa-2-azaheptadecan-17-oic acid

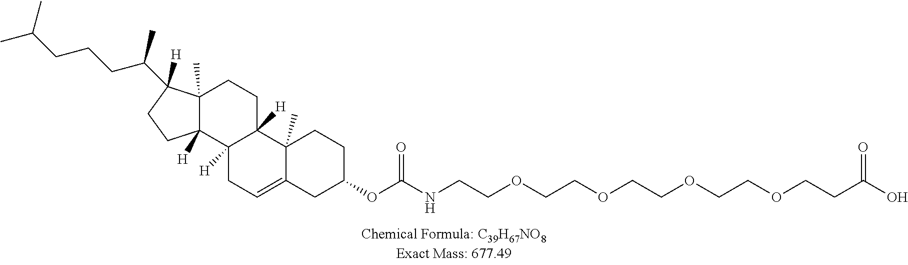 [SMILES: CC(C)CCC[C@@H](C)[C@H]1CC[C@H]2[C@@H]3CC=C4C[C@@H](OC(=O)NCCOCCOCCOCCOCCC(=O)O)CC[C@]4(C)[C@H]3CC[C@]12C]

A solution of tert-butyl 1-[(3β)-cholest-5-en-3-yloxy]-1-oxo-5,8,11,14-tetraoxa-2-azaheptadecan-17-oate (7.33 g, 9.98 mmol) in DCM (100 mL) was treated with TFA (40 mL). The reaction mixture was stirred at rt for 2 h. The reaction was then diluted with DCM (100 mL) and concentrated in vacuo to give a yellow gum. The crude product was lyophilized to give the title compound as a brown gum (7.78 g, >99%). The material was used in the next step without further purification.

¹H NMR (400 MHz, CDCl₃) δ 6.43 (br s, 1H), 5.28-5.32 (m, 1H), 4.44 (br s, 1H), 3.74 (t, 2H), 3.56-3.68 (m, 14H), 3.49-3.56 (t, 2H), 3.29-3.32 (m, 2H), 2.61 (t, 2H), 2.26 (br s, 2H), 1.67-1.99 (m, 5H), 0.82-1.58 (m, 29H), 0.75-0.82 (m, 6H), 0.60 (s, 3H).

3 extra H

### Step (iii): 7-bromo-3-nitro-N-[(2,2,5-trimethyl-1,3-dioxan-5-yl)methyl]quinolin-4-amine

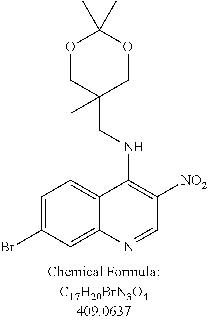 [SMILES: CC1(CNc2c([N+](=O)[O-])cnc3cc(Br)ccc23)COC(C)(C)OC1]

A suspension of 7-bromo-4-chloro-3-nitroquinoline (CAS: 723280-98-6, 28.1 g, 97.7 mmol) and triethylamine (Et₃N) (19.8 g, 195 mmol) in DCM (478 mL) was treated with a solution of (2,2,5-trimethyl-1,3-dioxan-5-yl)methanamine (CAS: 4933-20-4, 15.5 g, 97.7 mmol) in DCM (15 mL) at 10° C. The reaction mixture was stirred at rt for 3 h. The reaction mixture was diluted with DCM (300 mL), washed with brine (2×200 mL), dried over sodium sulfate, filtered and concentrated in vacuo. The resulting residue was suspended in DCM/petroleum ether (1:2, 150 mL) and stirred at rt for 20 min. The solids were collected by filtration to give the title compound (35.9 g, 89%) as a yellow solid. The material was used in the next step without further purification.

### MS(ES+): 411.9 (M+H⁺).

### Step (iv): 7-bromo-N-4-[(2,2,5-trimethyl-1,3-dioxan-5-yl)methyl]quinoline-3,4-diamine

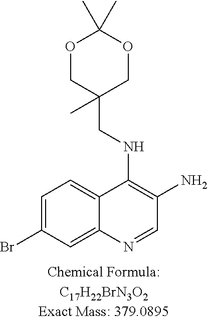 [SMILES: CC1(CNc2c(N)cnc3cc(Br)ccc23)COC(C)(C)OC1]

Reaction was performed in two batches: To a suspension of 7-bromo-3-nitro-N-[(2,2,5-trimethyl-1,3-dioxan-5-yl)methyl]quinolin-4-amine (34.5 g, 84.1 mmol) in THF (300 mL) was added 5% Pt/C (4.92 g, 1.26 mmol). The reaction mixture was stirred under a balloon of hydrogen (1 atm) at rt for 20 h. The two reaction mixtures were combined and filtered through a short pad of celite, washing with THF (1.2 L). The filtrate was concentrated in vacuo and the resulting residue was suspended in MeCN (120 mL) and stirred at rt for 15 min. The solids were collected by filtration and washed with MeCN (2×10 mL). The solids were dried under reduced pressure to give the title compound (45.2 g, 71%) as a yellow-green solid. The material was used in the next step without further purification.

### MS(ES+): 382.1 (M+H⁺).

### Step (v): N-(7-bromo-4-{[(2,2,5-trimethyl-1,3-dioxan-5-yl)methyl]amino}quinolin-3-yl)pentanamide

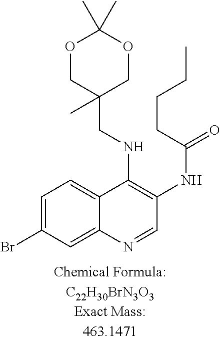 [SMILES: CCCCC(=O)Nc1cnc2cc(Br)ccc2c1NCC1(C)COC(C)(C)OC1]

A solution of 7-bromo-N-4-[(2,2,5-trimethyl-1,3-dioxan-5-yl)methyl]quinoline-3,4-diamine (50.5 g, 133 mmol) in DCM (70 mL) was treated with pentanoyl chloride (16 g, 133 mmol) dropwise at 0° C. The reaction mixture was allowed to slowly warm to rt over 2 h. The reaction mixture was poured into saturated aqueous sodium bicarbonate (1.2 L) and extracted with DCM (2×400 mL). The combined organic layers were washed with brine (500 mL), dried over sodium sulfate, filtered and concentrated under reduced pressure. The residue was filtered through a short plug of silica gel, eluting with ethyl acetate (EtOAc)/petroleum ether (1:2, 500 mL then 1:1, 500 mL) followed by ethyl acetate (EtOAc) (6 L). The filtrate was concentrated in vacuo to give the title compound (55.5 g, 90%) as an off-white solid.

### MS(ES+): 465.9 (M+H⁺).

### Step (vi): 7-bromo-2-butyl-1-[(2,2,5-trimethyl-1,3-dioxan-5-yl)methyl]-1H-imidazo[4,5-c]quinoline

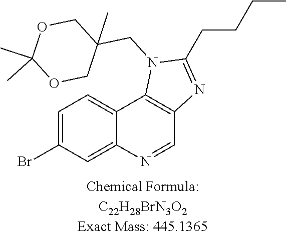 [SMILES: CCCCc1nc2cnc3cc(Br)ccc3c2n1CC1(C)COC(C)(C)OC1]

A mixture of N-(7-bromo-4-{[(2,2,5-trimethyl-1,3-dioxan-5-yl)methyl]amino}quinolin-3-yl)pentanamide (55.5 g, 119 mmol) in ethanol (EtOH) (500 mL) was treated with sodium hydroxide (59.8 g, 179 mmol). The reaction mixture was stirred at 90° C. in an oil batch for 20 h (note: internal temperature of the reaction mixture was 78° C.). The reaction mixture was concentrated in vacuo to remove most of the ethanol, diluted with water (300 mL), and stirred at rt for 10 min. The solids were collected by filtration and washed with water (3×10 mL). The filter cake was diluted with EtOAc/petroleum ether (2:1, 150 mL) and stirred at rt for 40 min. The solids were collected by filtration and washed with EtOAc (3×10 mL). The filter cake was diluted with MeCN (10 mL) and concentrated in vacuo to give the title compound (50 g, 93%) as a light yellow solid. The material was used in the next step without further purification.

### MS(ES+): 447.9 (M+H⁺).

### Step (vii): 7-bromo-2-butyl-1-[(2,2,5-trimethyl-1,3-dioxan-5-yl)methyl]-1H-imidazo[4,5-c]quinoline 5-oxide

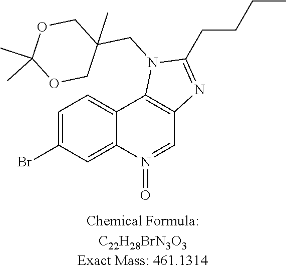 [SMILES: CCCCc1nc2c[n+]([O-])c3cc(Br)ccc3c2n1CC1(C)COC(C)(C)OC1]

In two separate round bottom flasks, a solution of 7-bromo-2-butyl-1-[(2,2,5-trimethyl-1,3-dioxan-5-yl)methyl]-1H-imidazo[4,5-c]quinoline (25 g, 56 mmol) in DCM (500 mL) was treated with m-CPBA (24.2 g, 112 mmol) and stirred at rt for 36 h. The reaction mixtures were combined, further diluted with DCM (500 mL), washed with saturated aqueous sodium bicarbonate (1.2 L) and brine (500 mL), dried over sodium sulfate and filtered. The organic solvents were removed under reduced pressure and the resulting residue was diluted with DCM/EtOAc/petroleum ether (20 mL/20 mL/100 mL) and stirred at rt for 1 h. The product was collected by filtration and the solids were washed with EtOAc/petroleum ether (1:1, 50 mL). The filter cake was then suspended in MeCN (120 mL) and stirred at rt for 20 min. The solids were collected by filtration and dried under reduced pressure to give the title compound (24 g, 46%) as a yellow solid. The material was used in the next step without further purification.

### MS(ES+): 463.9 (M+H⁺).

### Step (viii): 7-bromo-2-butyl-1-[(2,2,5-trimethyl-1,3-dioxan-5-yl)methyl]-1H-imidazo[4,5-c]quinolin-4-amine

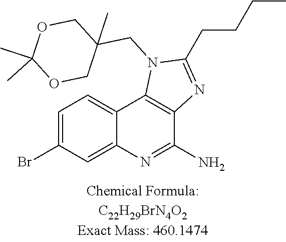 [SMILES: CCCCc1nc2c(N)nc3cc(Br)ccc3c2n1CC1(C)COC(C)(C)OC1]

A solution of 7-bromo-2-butyl-1-[(2,2,5-trimethyl-1,3-dioxan-5-yl)methyl]-1H-imidazo[4,5-c]quinoline 5-oxide (36.0 g, 77.8 mmol) in DCM (505 mL) was treated with ammonium hydroxide (101 mL) at 0° C. followed by tosyl chloride (TsCl) (17.8 g, 93.4 mmol). The reaction mixture was allowed to warm to rt and stirred for 3 h. The reaction mixture was then diluted with DCM (600 mL) and washed with saturated aqueous sodium bicarbonate (2×800 mL), dried over sodium sulfate, filtered and concentrated. The residue was diluted with EtOAc (100 mL) and stirred at rt for 20 min. The product was collected by filtration, and washed with EtOAc (2×20 mL). The filter cake was dried under reduced pressure to give the title compound (30.8 g, 86%) as a light yellow solid. The material was used in the next step without further purification.

### MS(ES+): 463.0 (M+H⁺).

### Step (ix): tert-butyl 4-(3-{4-amino-2-butyl-1-[(2,2,5-trimethyl-1,3-dioxan-5-yl)methyl]-1H-imidazo[4,5-c]quinolin-7-yl}prop-2-yn-1-yl)piperazine-1-carboxylate

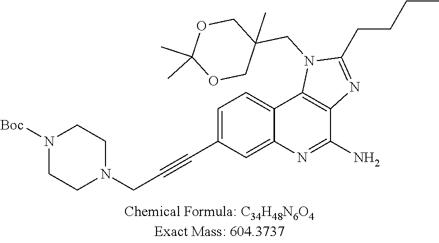 [SMILES: CCCCc1nc2c(N)nc3cc(C#CCN4CCN(C(=O)OC(C)(C)C)CC4)ccc3c2n1CC1(C)COC(C)(C)OC1]

A solution of 7-bromo-2-butyl-1-[(2,2,5-trimethyl-1,3-dioxan-5-yl)methyl]-1H-imidazo[4,5-c]quinolin-4-amine (10.3 g, 22.3 mmol) and tert-butyl 4-(prop-2-yn-1-yl)piperazine-1-carboxylate (CAS: 199538-99-3, 6.01 g, 26.8 mmol) in DMF (200 mL) was added Pd(PPh₃)₄ (2.58 g, 2.23 mmol), CuI (850 mg, 4.46 mmol), and Cs₂CO₃ (36.4 g, 112 mmol). The mixture was degassed with argon and stirred at 85° C. for 16 h. The reaction mixture was cooled to rt and treated with water (800 mL) and stirred for 15 min. The solids were collected by filtration and washed with water (2×50 mL). The aqueous filtrates were discarded and the filter cake was flushed with DCM (100 mL). The resulting filtrate was dried over sodium sulfate, filtered and concentrated to give a brown gum. The crude product was suspended in MeCN (50 mL) and stirred for 15 min. The solids were collected by filtration and washed with additional MeCN (2×5 mL) to give the title compound (8.0 g, 59%) as an off-white solid. The material was used in the next step without further purification.

### MS(ES+): 605.4 (M+H⁺).

### Step (x): tert-butyl 4-(3-{4-amino-2-butyl-1-[(2,2,5-trimethyl-1,3-dioxan-5-yl)methyl]-1H-imidazo[4,5-c]quinolin-7-yl}propyl)piperazine-1-carboxylate

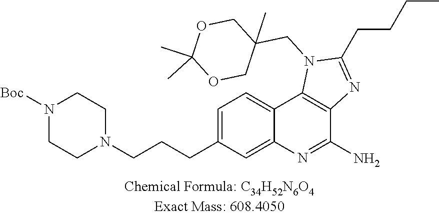 [SMILES: CCCCc1nc2c(N)nc3cc(CCCN4CCN(C(=O)OC(C)(C)C)CC4)ccc3c2n1CC1(C)COC(C)(C)OC1]

A mixture of tert-butyl 4-(3-{4-amino-2-butyl-1-[(2,2,5-trimethyl-1,3-dioxan-5-yl)methyl]-1H-imidazo[4,5-c]quinolin-7-yl}prop-2-yn-1-yl)piperazine-1-carboxylate (1.2 g, 2.0 mmol) and 10% Pd/C (400 mg) in THF (30 mL) was degassed with hydrogen three times, then stirred under a balloon of hydrogen (1 atm) for 20 h. The reaction mixture was filtered through a plug of celite, washing with MeOH (250 mL). The filtrate was concentrated and purified using column chromatography (silica, DCM/MeOH, 100:0 to 9:1) to give the title compound (1.2 g, >99%) as a brown solid.

### MS(ES+): 609.4 (M+H⁺).

### Step (xi): 2-({4-amino-2-butyl-7-[3-(piperazin-1-yl)propyl]-1H-imidazo[4,5-c]quinolin-1-yl}methyl)-2-methylpropane-1,3-diol

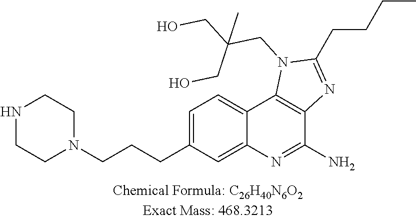 [SMILES: CCCCc1nc2c(N)nc3cc(CCCN4CCNCC4)ccc3c2n1CC(C)(CO)CO]

To a solution of tert-butyl 4-(3-{4-amino-2-butyl-1-[(2,2,5-trimethyl-1,3-dioxan-5-yl)methyl]-1H-imidazo[4,5-c]quinolin-7-yl}propyl)piperazine-1-carboxylate (9.4 g, 15.4 mmol) in DCM (50 mL) and MeOH (50 mL) was added a solution of HCl in EtOAc (4M, 100 mL). The mixture was stirred at rt for 4 h. The reaction mixture was concentrated in vacuo to give the title compound (9.7 g) as a light yellow solid. The material was used in the next step without further purification.

### MS(ES+): 469.2 (M+H⁺).

### Step (xii): tert-butyl {75-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-75-oxo-3,6,9,12,15,18,21,24,27,30,33,36,39,42,45,48,51,54,57,60,63,66,69,72-tetracosaoxapentaheptacont-1-yl}carbamate

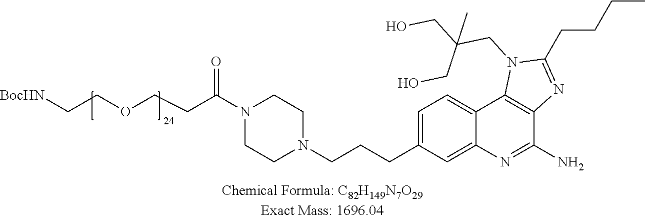

A solution of 2,2-dimethyl-4-oxo-3,8,11,14,17,20,23,26,29,32,35,38,41,44,47,50,53,56,59,62,65,68,71,74,77-pentacosaoxa-5-azaoctacontan-80-oic acid (1.5 g, 1.2 mmol), and 2-({4-amino-2-butyl-7-[3-(piperazin-1-yl)propyl]-1H-imidazo[4,5-c]quinolin-1-yl}methyl)-2-methylpropane-1,3-diol (717 mg, 1.32 mmol) in DMF (50 mL) and DCM (10 mL) was treated with HATU (503 mg, 1.32 mmol) and DIPEA (1.09 g, 8.42 mmol) at 0° C. The reaction mixture was warmed to rt and stirred for 16 h. The reaction mixture was concentrated in vacuo and dissolved in ethyl acetate (EtOAc) (30 mL). The solution was filtered through a short pad of silica gel, eluting with additional ethyl acetate (EtOAc) (200 mL) followed by DCM/MeOH (9:1, 100 mL). The filtrates were discarded. The plug of silica gel was flushed with DCM/MeOH/ammonium hydroxide (10:1:0.3), which caused the desired product to elute from the silica gel. The filtrate was concentrated in vacuo to give the title compound (2.6 g, >99%) as a yellow oil.

### MS(ES+): 799.4 (M-Boc/2+H⁺).

### Step (xiii): 1-amino-75-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-3,6,9,12,15,18,21,24,27,30,33,36,39,42,45,48,51,54,57,60,63,66,69,72-tetracosaoxapentaheptacontan-75-one

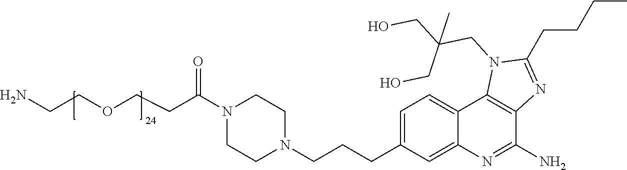

A solution of tert-butyl {75-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-75-oxo-3,6,9,12,15,18,21,24,27,30,33,36,39,42,45,48,51,54,57,60,63,66,69,72-tetracosaoxapentaheptacont-1-yl}carbamate (2.6 g, 1.3 mmol) in DCM (35 mL) was treated with TFA (15 mL). The reaction mixture was stirred at rt for 1 h and then concentrated in vacuo. The crude product was lyophilized for 72 h to give a crude yellow oil (3 g). The material was used in the next step without further purification.

MS(ES+): 533.1 (M/3+H⁺), 799.4 (M/2+H⁺).

### Step (xiv): (3β)-cholest-5-en-3-yl {91-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-15,91-dioxo-3,6,9,12,19,22,25,28,31,34,37,40,43,46,49,52,55,58,61,64,67,70,73,76,79,82,85,88-octacosaoxa-16-azahennonacont-1-yl}carbamate (Compound 1)

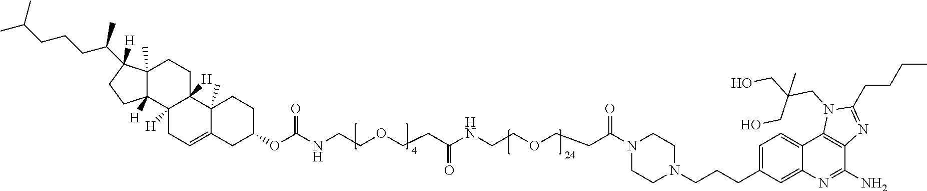

A solution of 1-[(3β)-cholest-5-en-3-yloxy]-1-oxo-5,8,11,14-tetraoxa-2-azaheptadecan-17-oic acid (from Example 1, step ii)(1.15 g, 1.40 mmol) and 1-amino-75-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-3,6,9,12,15,18,21,24,27,30,33,36,39,42,45,48,51,54,57,60,63,66,69,72-tetracosaoxapentaheptacontan-75-one (3.0 g, 1.55 mmol) in DMF (15 mL) was added HATU (577 mg, 1.52 mmol) at 0° C. followed by DIPEA (1.87 g, 14.5 mmol). The reaction mixture was allowed to warm to rt and stirred for 16 h. The reaction mixture was concentrated in vacuo and the residue was purified using prep HPLC (Boston Prime C18 150*30 mm*5 uM, eluting with A:water (10 mM ammonium hydroxide)/B:(MeCN/THF, 1:1), A/B: 53/47 to 33/77 over 8 min, 25 mL/min, 26 injections). The desired product was repurified using prep HPLC (Phenomenex Gemini NX C18 150*30 mm*5 um, eluting with A: water (10 mM NH₄HCO₃)/B: (MeCN/THF (1:1)), A/B: 60/40 to 5/95, 30 mg per injection) to give the title compound (340 mg, 10%) as a white solid.

MS(ES+): 753.4 (M/3+H⁺), 1129.3 (M/2+H⁺).

¹H NMR (400 MHz, DMSO-d₆) δ 8.42 (d, 1H), 7.89 (t, 1H), 7.40 (s, 1H), 6.96-7.16 (m, 2H), 6.38 (br s, 2H), 5.33 (br s, 1H), 4.97 (br s, 2H), 4.75 (br s, 1H), 4.44 (br s, 1H), 4.25-4.36 (m, 1H), 3.47-3.64 (m, 112H), 3.42-3.46 (m, 6H), 3.32-3.42 (m, 16H), 3.11 (q, 2H), 3.20 (q, 3H), 2.96 (br s, 1H), 2.70 (t, 2H), 2.55 (t, 2H), 2.15-2.39 (m, 10H), 1.70-2.00 (m, 9H), 0.87-1.58 (m, 34H), 0.82-0.90 (m, 6H), 0.65 (s, 3H), 0.55 (s, 3H).

### Example 2

### (3 beta)-cholest-5-en-3-yl ((6S,9S)-1-amino-6-((4-((4-(3-(4-amino-2-butyl-1-(3-hydroxy-2-(hydroxymethyl)-2-methylpropyl)-1H-imidazo[4,5-c]quinolin-7-yl)propyl)piperazin-1-yl)methyl)phenyl)carbamoyl)-9-isopropyl-1,8,11-trioxo-13,16-dioxa-2,7,10-triazaoctadecan-18-yl)carbamate

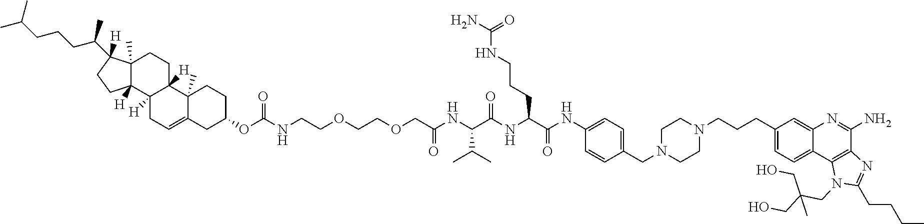 [SMILES: CCCCc1nc2c(N)nc3cc(CCCN4CCN(Cc5ccc(NC(=O)[C@H](CCCNC(N)=O)NC(=O)[C@@H](NC(=O)COCCOCCNC(=O)O[C@H]6CC[C@@]7(C)C(=CC[C@H]8[C@@H]9CC[C@H]([C@H](C)CCCC(C)C)[C@@]9(C)CC[C@@H]87)C6)C(C)C)cc5)CC4)ccc3c2n1CC(C)(CO)CO]

### Step (i): tert-butyl 2-(2-(2-[(3beta)-cholest-5-en-3-yloxy]carbonyl)amino)ethoxy)ethoxy)acetate

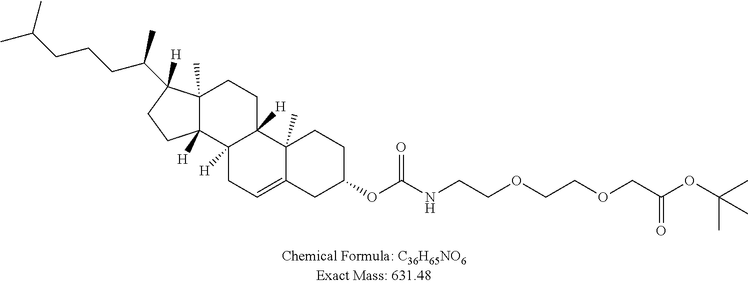 [SMILES: CC(C)CCC[C@@H](C)[C@H]1CC[C@H]2[C@@H]3CC=C4C[C@@H](OC(=O)NCCOCCOCC(=O)OC(C)(C)C)CC[C@]4(C)[C@H]3CC[C@]12C]

To a solution of tert-butyl 2-(2-(2-aminoethoxy)ethoxy)acetate (CAS: 1122484-77-8, 3.00 g, 13.7 mmol) in DCM (90 mL) and Et₃N (2.77 g, 27.4 mmol) at 0-5° C. under a nitrogen atmosphere, was added cholesteryl chloroformate (CAS: 7144-08-3, 6.14 g, 13.7 mmol). The reaction was warmed to rt and stirred for 16 h. The reaction mixture was diluted with brine (100 mL) then extracted with DCM. The organic layer was concentrated in vacuo to give a white gum residue which was purified by column chromatography (silica, DCM/MeOH, 100:0 to 9:1) to give the title compound (8.52 g, 98%) as a white gum.

¹H NMR (400 MHz, CDCl₃) δ 5.32-5.39 (m, 1H), 5.14-5.20 (m, 1H), 4.43-4.55 (m, 1H), 3.98-4.03 (m, 2H), 3.67-3.71 (m, 2H), 3.62-3.66 (m, 2H), 3.56 (t, 2H), 3.37 (q, 2H), 2.21-2.41 (m, 2H), 1.74-2.02 (m, 6H), 1.64 (s, 1H), 1.51-1.58 (m, 3H), 1.46-1.49 (m, 9H), 1.21-1.45 (m, 5H), 0.92-1.20 (m, 14H), 0.91 (d, 3H), 0.86 (dd, 6H), 0.67 (s, 3H).

### Step (ii): 2-(2-(2-[(3beta)-cholest-5-en-3-yloxy]carbonyl)amino)ethoxy)ethoxy)acetic acid

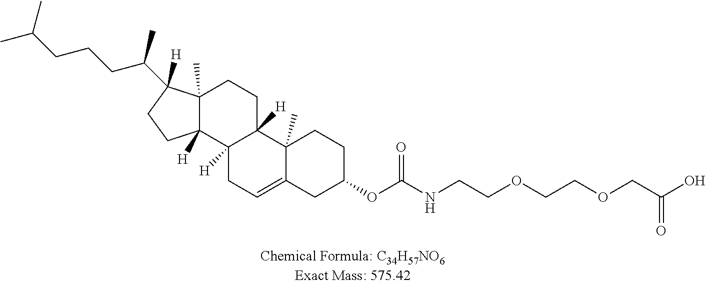 [SMILES: CC(C)CCC[C@@H](C)[C@H]1CC[C@H]2[C@@H]3CC=C4C[C@@H](OC(=O)NCCOCCOCC(=O)O)CC[C@]4(C)[C@H]3CC[C@]12C]

A solution of tert-butyl 2-(2-(2-[(3beta)-cholest-5-en-3-yloxy]carbonyl)amino)ethoxy)ethoxy)acetate (8.52 g, 13.5 mmol) in DCM (100 mL) was treated with TFA (50 mL) at rt under nitrogen atmosphere. The reaction mixture was stirred at rt for 3 h then concentrated in vacuo. The dark green gum was then diluted with water (100 mL) then stirred for 20 min. The green gum became a yellow solid which was filtered then the filter cake was lyophilized to give the title compound as a light yellow solid (7.32 g, 94%). The material was used in the next step without further purification.

¹H NMR (400 MHz, CDCl₃) δ 5.33-5.45 (m, 1H), 5.02-5.12 (m, 1H), 4.43-4.58 (m, 2H), 4.17 (s, 2H), 3.74-3.77 (m, 2H), 3.64-3.68 (m, 2H), 3.59 (t, 2H), 3.32-3.42 (m, 2H), 2.21-2.49 (m, 2H), 1.76-2.05 (m, 5H), 0.94-1.62 (m, 24H), 0.91 (d, 3H), 0.86 (dd, 6H), 0.67 (s, 3H).

### Step (iii): [(3beta)-cholest-5-en-3-yloxy]((6S,9S)-1-amino-6-((4-(hydroxymethyl)phenyl)carbamoyl)-9-isopropyl-1,8,11-trioxo-13,16-dioxa-2,7,10-triazaoctadecan-18-yl)carbamate

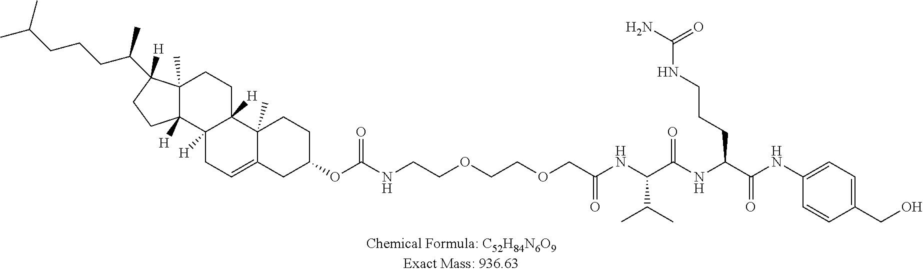 [SMILES: CC(C)CCC[C@@H](C)[C@H]1CC[C@H]2[C@@H]3CC=C4C[C@@H](OC(=O)NCCOCCOCC(=O)N[C@H](C(=O)N[C@@H](CCCNC(N)=O)C(=O)Nc5ccc(CO)cc5)C(C)C)CC[C@]4(C)[C@H]3CC[C@]12C]

To a solution of (S)-2-((S)-2-amino-3-methylbutanamido)-N-(4-(hydroxymethyl)phenyl)-5-ureidopentanamide (CAS: 2247291-44-5, 105 mg, 0.278 mmol) and 2-(2-(2-[(3beta)-cholest-5-en-3-yloxy]carbonyl)amino)ethoxy)ethoxy)acetic acid (0.200 g, 0.278 mmol) in DMF (5 mL) was added HATU (0.106 g, 0.278 mmol) then DIPEA (215 mg, 1.67 mmol) at 0° C. under nitrogen atmosphere. The reaction was warmed to rt then stirred for 16 h. The mixture was concentrated in vacuo. The residue was added dropwise to ice water (10 mL) which caused solid to form. The solid was filtered then washed with water (5 mL) and concentrated in vacuo to give a white solid residue. The residue was purified by column chromatography (silica, DCM/MeOH, 100:0 to 4:1) to give the title compound (0.130 g, 50%) as a white solid.

### MS(ES+): 959.6 [(M+1)+23]

### Step (iv): [(3beta)-cholest-5-en-3-yloxy]((6S,9S)-1-amino-6-((4-(iodomethyl)phenyl)carbamoyl)-9-isopropyl-1,8,11-trioxo-13,16-dioxa-2,7,10-triazaoctadecan-18-yl)carbamate

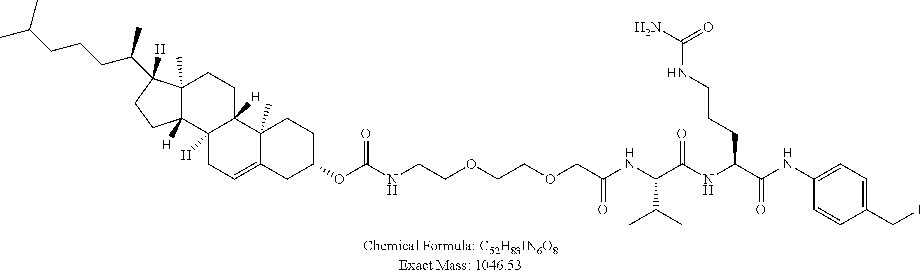 [SMILES: CC(C)CCC[C@@H](C)[C@H]1CC[C@H]2[C@@H]3CC=C4C[C@@H](OC(=O)NCCOCCOCC(=O)N[C@H](C(=O)N[C@@H](CCCNC(N)=O)C(=O)Nc5ccc(CI)cc5)C(C)C)CC[C@]4(C)[C@H]3CC[C@]12C]

At 0° C., to a solution of [(3beta)-cholest-5-en-3-yloxy]((6S,9S)-1-amino-6-((4-(hydroxymethyl)phenyl)carbamoyl)-9-isopropyl-1,8,11-trioxo-13,16-dioxa-2,7,10-triazaoctadecan-18-yl)carbamate (0.130 g, 0.139 mmol) in DCM (4 mL) was added sodium iodide (62.4 mg, 0.416 mmol) followed by the slow addition of a solution of TMSCl (45.2 mg, 0.416 mmol) in DCM (0.5 mL). The reaction was warmed to rt then stirred for 2 h. The mixture was diluted with water (10 mL) and extracted with DCM (10 mL×2). The combined organic layer was concentrated in vacuo to a yellow solid which was dissolved in MeCN (4 mL) and stirred for 20 min. The mixture was filtered then washed with MeCN to give the title compound (0.120 g, 83%) as a yellow solid. The material was used in the next step without further purification.

### Step (v): (3beta)-cholest-5-en-3-yl ((6S,9S)-1-amino-6-((4-((4-(3-(4-amino-2-butyl-1-(3-hydroxy-2-(hydroxymethyl)-2-methylpropyl)-1H-imidazo[4,5-c]quinolin-7-yl)propyl)piperazin-1-yl)methyl)phenyl)carbamoyl)-9-isopropyl-1,8,11-trioxo-13,16-dioxa-2,7,10-triazaoctadecan-18-yl)carbamate

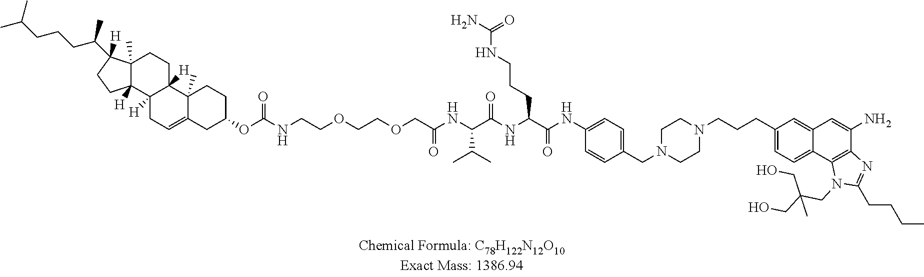 [SMILES: CCCCc1nc2c(N)cc3cc(CCCN4CCN(Cc5ccc(NC(=O)[C@H](CCCNC(N)=O)NC(=O)[C@@H](NC(=O)COCCOCCNC(=O)O[C@H]6CC[C@@]7(C)C(=CC[C@H]8[C@@H]9CC[C@H]([C@H](C)CCCC(C)C)[C@@]9(C)CC[C@@H]87)C6)C(C)C)cc5)CC4)ccc3c2n1CC(C)(CO)CO]

The reaction was conducted in two batches then combined for purification. To a mixture of 2-({4-amino-2-butyl-7-[3-(piperazin-1-yl)propyl]-1H-imidazo[4,5-c]quinolin-1-yl}methyl)-2-methylpropane-1,3-diol (from Example 1, step xi) (0.030 g, 0.052 mmol), [(3beta)-cholest-5-en-3-yloxy]((6S,9S)-1-amino-6-((4-(iodomethyl)phenyl)carbamoyl)-9-isopropyl-1,8,11-trioxo-13,16-dioxa-2,7,10-triazaoctadecan-18-yl)carbamate (120 mg, 0.069 mmol) in DMF (2 mL) was added K₂CO₃ (28.7 mg, 0.208 mmol) and DIPEA (26.8 mg, 0.208 mmol). The reaction was heated to 80° C. and stirred for 5 h. Ice water (5 mL) was added to the reaction which caused solid to form. The solid residue was filtered then washed with water and concentrated in vacuo to provide a white solid. A second batch of the same reaction was conducted with 2-({4-amino-2-butyl-7-[3-(piperazin-1-yl)propyl]-1H-imidazo[4,5-c]quinolin-1-yl}methyl)-2-methylpropane-1,3-diol (from Example 1, step xi) (25 mg, 0.043 mmol). The batches were combined then concentrated in vacuo to form a yellow solid that was purified by prep HPLC (C18, 150*30 mm*5 um, eluting with A: water (10 mM NH₄HCO₃), B: MeCN/THF (1:1); A %/B %: 60/40 to 35/65 over 16 min, held at 100% B for 7 min, Flow 30 mL/min, 8 injections) and lyophilized to give the title compound (61.7 mg, 47%) as a white solid.

### MS(ES+): 1388.1 (M+H⁺).

¹H NMR (400 MHz, MeOD-d₄) δ 8.53 (d, 1H), 7.86 (t, 1H), 7.48-7.56 (m, 3H), 7.17-7.32 (m, 3H), 5.29-5.37 (m, 1H), 4.90-4.97 (m, 2H), 4.49-4.67 (m, 4H), 4.27-4.38 (m, 2H), 4.04 (s, 2H), 3.88-3.94 (m, 1H), 3.52-3.77 (m, 9H), 3.49 (s, 2H), 3.37-3.46 (m, 1H), 3.01-3.25 (m, 4H), 2.76 (t, 2H), 2.37-2.61 (m, 7H), 2.19-2.30 (m, 2H), 2.06-2.17 (m, 1H), 1.66-2.04 (m, 12H), 1.29-1.63 (m, 14H), 0.85-1.26 (m, 31H), 0.60-0.72 (m, 6H).

### Example 3

### 4-((12S,15S)-1-(((3beta)-cholest-5-en-3-yl)oxy)-12-isopropyl-1,10,13-trioxo-15-(3-ureidopropyl)-5,8-dioxa-2,11,14-triazahexadecan-16-amido)benzyl 4-(3-(4-amino-2-butyl-1-(3-hydroxy-2-(hydroxymethyl)-2-methylpropyl)-1H-imidazo[4,5-c]quinolin-7-yl)propyl)piperazine-1-carboxylate

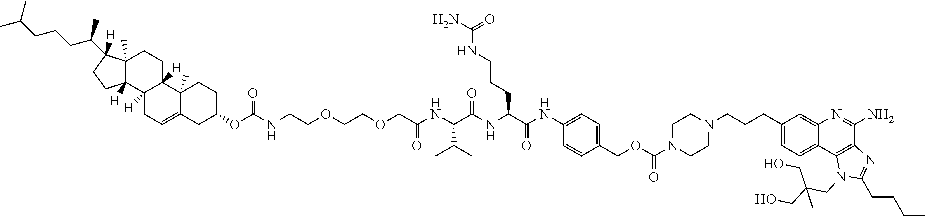 [SMILES: CCCCc1nc2c(N)nc3cc(CCCN4CCN(C(=O)OCc5ccc(NC(=O)[C@H](CCCNC(N)=O)NC(=O)[C@@H](NC(=O)COCCOCCNC(=O)O[C@H]6CC[C@@]7(C)C(=CC[C@H]8[C@@H]9CC[C@H]([C@H](C)CCCC(C)C)[C@@]9(C)CC[C@@H]87)C6)C(C)C)cc5)CC4)ccc3c2n1CC(C)(CO)CO]

### Step (i). [(3beta)-cholest-5-en-3-yloxy]((6S,9S)-1-amino-9-isopropyl-6-((4-((((4-nitrophenoxy)carbonyl)oxy)methyl)phenyl)carbamoyl)-1,8,11-trioxo-13,16-dioxa-2,7,10-triazaoctadecan-18-yl)carbamate

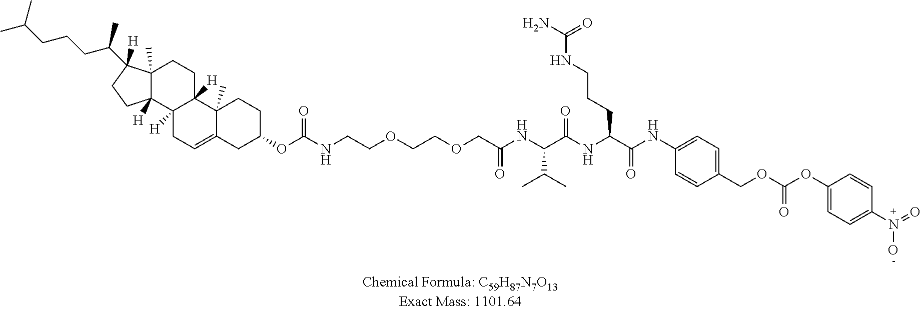

At 0-5° C., to a stirred solution of [(3beta)-cholest-5-en-3-yloxy]((6S,9S)-1-amino-6-((4-(hydroxymethyl)phenyl)carbamoyl)-9-isopropyl-1,8,11-trioxo-13,16-dioxa-2,7,10-triazaoctadecan-18-yl)carbamate (from Example 2, step iii) (0.850 g, 0.907 mmol) in DMF (20 mL) was added bis(4-nitrophenyl)carbonate (CAS: 5070-13-3, 0.828 g, 2.72 mmol) followed by DIPEA (0.293 g, 2.27 mmol) under nitrogen atmosphere. The yellow clear reaction was stirred at rt for 16 h. The mixture was poured into the ice water then extracted with DCM (20 mL×2). The combined organic layer was washed with brine then the organic layer was concentrated in vacuo to give a yellow gum residue. The residue was purified by column chromatography (silica, DCM/MeOH, 100:0 to 4:1) to give the title compound (0.670 g, 67%) as a white solid.

### MS(ES+): 1102.6 (M+H⁺).

¹H NMR (400 MHz, DMSO-d₆) δ 10.12 (s, 1H), 8.37 (d, 1H), 8.30 (dt, 2H), 7.64 (d, 2H), 7.56 (dt, 2H), 7.45 (d, 1H), 7.40 (d, 2H), 7.03 (t, 1H), 5.99 (t, 1H), 5.42 (br s, 2H), 5.28-5.32 (m, 1H), 5.23 (s, 2H), 4.21-4.45 (m, 3H), 3.94 (s, 2H), 3.50-3.65 (m, 4H), 3.41 (t, 2H), 3.12 (q, 2H), 2.87-3.08 (m, 2H), 2.12-2.39 (m, 2H), 1.83-2.07 (m, 3H), 1.57-1.80 (m, 5H), 1.40-1.53 (m, 6H), 1.26-1.39 (m, 6H), 1.15-1.25 (m, 1H), 0.94-1.14 (m, 9H), 0.90-0.94 (m, 3H), 0.85-0.89 (m, 6H), 0.79-0.84 (m, 10H), 0.62 (s, 3H).

### Step (ii). 4-((12S,15S)-1-(((3beta)-cholest-5-en-3-yl)oxy)-12-isopropyl-1,10,13-trioxo-15-(3-ureidopropyl)-5,8-dioxa-2,11,14-triazahexadecan-16-amido)benzyl 4-(3-(4-amino-2-butyl-1-(3-hydroxy-2-(hydroxymethyl)-2-methylpropyl)-1H-imidazo[4,5-c]quinolin-7-yl)propyl)piperazine-1-carboxylate

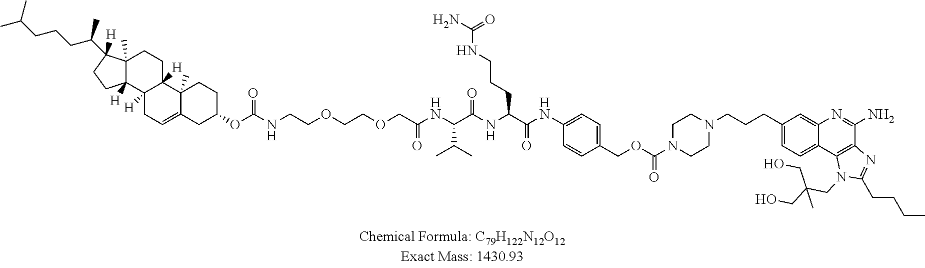 [SMILES: CCCCc1nc2c(N)nc3cc(CCCN4CCN(C(=O)OCc5ccc(NC(=O)[C@H](CCCNC(N)=O)NC(=O)[C@@H](NC(=O)COCCOCCNC(=O)O[C@H]6CC[C@@]7(C)C(=CC[C@H]8[C@@H]9CC[C@H]([C@H](C)CCCC(C)C)[C@@]9(C)CC[C@@H]87)C6)C(C)C)cc5)CC4)ccc3c2n1CC(C)(CO)CO]

At 0° C., to a solution of [(3beta)-cholest-5-en-3-yloxy]((6S,9S)-1-amino-9-isopropyl-6-((4-((((4-nitrophenoxy)carbonyl)oxy)methyl)phenyl)carbamoyl)-1,8,11-trioxo-13,16-dioxa-2,7,10-triazaoctadecan-18-yl)carbamate (0.15 g, 0.14 mmol) in DCM (2 mL) and DMF (2 mL) was added DMAP (33.8 mg, 0.277 mmol) followed by Et₃N (0.119 g, 1.18 mmol) under nitrogen atmosphere. To the reaction mixture was added 2-({4-amino-2-butyl-7-[3-(piperazin-1-yl)propyl]-1H-imidazo[4,5-c]quinolin-1-yl}methyl)-2-methylpropane-1,3-diol (from Example 1, step xi) (0.080 g, 0.14 mmol) at 0° C. The reaction was warmed to rt and stirred for 16 h. The mixture was quenched with water (10 mL) then extracted with the DCM (10 mL×2). The combined organic layer was concentrated in vacuo and the residue was purified twice by prep HPLC. The first purification was by prep HPLC (C18, 150*30 mm*5 um, eluting with A:water (10 mM NH₄HCO₃), B: MeCN/THF (1:1); A %/B %: 55/45 to 30/70 over 16 min, 100% B hold for 8 min, flow rate 30 mL/min, 10 injections) and lyophilized to give the title compound (160 mg, 81%) as a white solid. The second purification was by prep HPLC (Boston Prime C18, 150*30 mm*5 um, eluting with A:water (10 mM NH₄HCO₃), B: MeCN/THF (1:1); A %/B %: 55/45 to 30/70 over 8 min, 100% B hold for 3 min, flow rate 30 mL/min, 8 injections) to give the title compound (90 mg, 49%) as a white solid. The solid was purified again by SFC (DAICEL CHIRALPAK ID, 250*30 mm*10 um, eluting with A: MeOH, B: MeCN; A %/B %: 60/40. Flow 80 mL/min, 100 injections) to give the title compound (45 mg, 23%) as a white solid.

### MS(ES+): 1431.8 (M+H⁺).

¹H NMR (400 MHz, DMSO-d₆) δ 10.04 (s, 1H), 8.37-8.42 (m, 1H), 8.31-8.37 (m, 1H), 7.55-7.60 (m, 2H), 7.43-7.47 (m, 1H), 7.36-7.39 (m, 1H), 7.26-7.31 (m, 2H), 6.99-7.07 (m, 2H), 6.32 (s, 2H), 5.94-6.01 (m, 1H), 5.41 (s, 2H), 5.27-5.32 (m, 1H), 4.92-5.03 (m, 4H), 4.70-4.80 (m, 1H), 4.23-4.49 (m, 3H), 3.94 (s, 2H), 3.50-3.64 (m, 4H), 3.38-3.43 (m, 2H), 3.07-3.15 (m, 3H), 2.88-3.08 (m, 5H), 2.63-2.73 (m, 2H), 2.26-2.36 (m, 6H), 1.83-2.27 (m, 6H), 1.56-1.82 (m, 10H), 1.16-1.52 (m, 17H), 0.78-1.14 (m, 34H), 0.63 (s, 3H), 0.54 (s, 3H).

### Example 4

### N-{75-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-75-oxo-3,6,9,12,15,18,21,24,27,30,33,36,39,42,45,48,51,54,57,60,63,66,69,72-tetracosaoxapentaheptacont-1-yl}-17-oxo-20-({(2R)-2,5,7,8-tetramethyl-2-[(4R,8R)-4,8,12-trimethyltridecyl]-3,4-dihydro-2H-chromen-6-yl}oxy)-4,7,10,13-tetraoxa-16-azaicosan-1-amide (Compound 2)

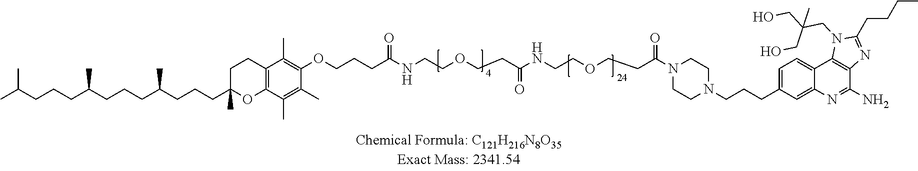

### Step (i): tert-butyl 4-({(2R)-2,5,7,8-tetramethyl-2-[(4R,8R)-4,8,12-trimethyltridecyl]-3,4-dihydro-2H-chromen-6-yl}oxy)butanoate

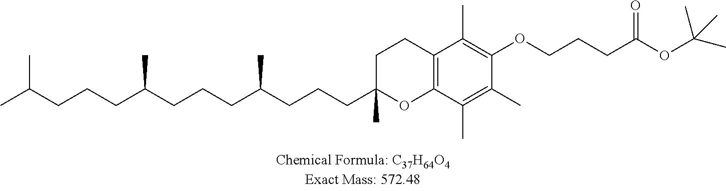 [SMILES: Cc1c(C)c2c(c(C)c1OCCCC(=O)OC(C)(C)C)CC[C@@](C)(CCC[C@H](C)CCC[C@H](C)CCCC(C)C)O2]

A mixture of (2R)-2,5,7,8-tetramethyl-2-[(4R,8R)-4,8,12-trimethyltridecyl]-3,4-dihydro-2H-chromen-6-ol (CAS: 59-02-9, 4.0 g, 9.3 mmol), Cs₂CO₃ (9.08 g, 27.9 mmol), TBAI (343 mg, 0.929 mmol) in DMF (80 mL) was treated with tert-butyl 4-bromobutanoate (CAS: 110661-91-1, 4.2 g, 18.8 mmol). The reaction mixture was heated at 85° C. for 4 h. The reaction mixture was poured into water (240 mL) then extracted with ethyl acetate (EtOAc) (3×50 mL). The combined organic layer was washed with 1N HCl (3×30 mL) then saturated aqueous sodium bicarbonate (NaHCO₃) (30 mL) and brine (40 mL). The organic layer was dried over sodium sulfate, filtered and concentrated in vacuo. The residue was purified using column chromatography (silica, petroleum ether/EtOAc, 100:0 to 95:5) to give the title compound (5.4 g, >99%) as a yellow gum.

¹H NMR (400 MHz, CDCl₃) δ 3.66 (t, 2H), 2.58 (t, 2H), 2.51 (t, 2H), 2.16 (s, 3H), 2.12 (s, 3H), 2.04-2.10 (m, 5H), 1.70-1.88 (m, 2H), 1.49-1.59 (m, 4H), 1.47 (s, 9H), 1.34-1.44 (m, 5H), 1.25-1.34 (m, 5H), 1.24 (s, 3H), 1.01-1.22 (m, 8H), 0.89 (s, 3H), 0.84-0.89 (m, 4H), 0.83-0.86 (m, 4H).

### Step (ii): 4-({(2R)-2,5,7,8-tetramethyl-2-[(4R,8R)-4,8,12-trimethyltridecyl]-3,4-dihydro-2H-chromen-6-yl}oxy)butanoic acid

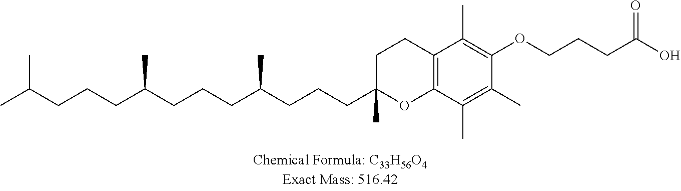 [SMILES: Cc1c(C)c2c(c(C)c1OCCCC(=O)O)CC[C@@](C)(CCC[C@H](C)CCC[C@H](C)CCCC(C)C)O2]

A solution of tert-butyl 4-({(2R)-2,5,7,8-tetramethyl-2-[(4R,8R)-4,8,12-trimethyltridecyl]-3,4-dihydro-2H-chromen-6-yl}oxy)butanoate (5.4 g, 9.42 mmol) in DCM (10 mL) was treated with TFA (50 mL) and stirred at rt for 1.5 h. The reaction mixture was concentrated in vacuo and lyophilized to give the title compound (4.4 g, 90%) as an off-white solid. The material was used in the next step without further purification.

¹H NMR (400 MHz, CDCl₃) δ 3.70 (t, 2H), 2.69 (t, 2H), 2.54-2.62 (m, 2H), 2.07-2.18 (m, 11H), 1.71-1.87 (m, 2H), 1.49-1.61 (m, 3H), 1.25-1.49 (m, 10H), 1.21-1.26 (m, 4H), 1.00-1.22 (m, 8H), 0.84-0.90 (m, 12H).

### Step (iii): tert-butyl 17-oxo-20-({(2R)-2,5,7,8-tetramethyl-2-[(4R,8R)-4,8,12-trimethyltridecyl]-3,4-dihydro-2H-chromen-6-yl}oxy)-4,7,10,13-tetraoxa-16-azaicosan-1-oate

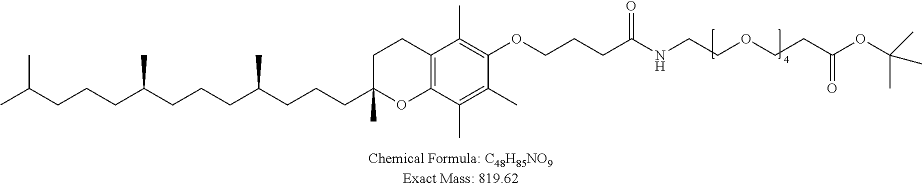

A mixture of tert-butyl 1-amino-3,6,9,12-tetraoxapentadecan-15-oate (4.4 g, 8.1 mmol) and HATU (3.1 g, 8.1 mmol) in DMF (35 mL) was added Hunig's base (DIPEA) (3.1 g, 24 mmol) followed by 4-({(2R)-2,5,7,8-tetramethyl-2-[(4R,8R)-4,8,12-trimethyltridecyl]-3,4-dihydro-2H-chromen-6-yl}oxy)butanoic acid (2.6 g, 8.1 mmol) as a solution in DMF (10 mL). The reaction mixture was stirred at rt for 16 h and poured into water (15 mL). The product was extracted with ethyl acetate (EtOAc) (3×50 mL). The combined organic layer was washed with 1N HCl (2×20 mL) then saturated sodium carbonate (2×20 mL) and brine (2×20 mL). The organic layer was dried over sodium sulfate, filtered, and the residue was purified using column chromatography (silica, DCM/MeOH, 100:0 to 90:10) to give the title compound (6.04 g, 91%) as a yellow gum.

¹H NMR (400 MHz, CDCl₃) δ 6.35 (br s, 1H), 3.63-3.73 (m, 16H), 3.58 (br t, 2H), 3.48 (q, 2H), 2.57 (t, 2H), 2.46-2.53 (m, 4H), 2.03-2.20 (m, 13H), 1.71-1.85 (m, 2H), 1.50-1.59 (m, 3H), 1.45 (s, 9H), 1.33-1.43 (m, 5H), 1.24-1.32 (m, 6H), 1.23 (s, 3H), 1.01-1.19 (m, 8H), 0.82-0.90 (m, 13H).

### Step (iv): 17-oxo-20-({(2R)-2,5,7,8-tetramethyl-2-[(4R,8R)-4,8,12-trimethyltridecyl]-3,4-dihydro-2H-chromen-6-yl}oxy)-4,7,10,13-tetraoxa-16-azaicosan-1-oic acid

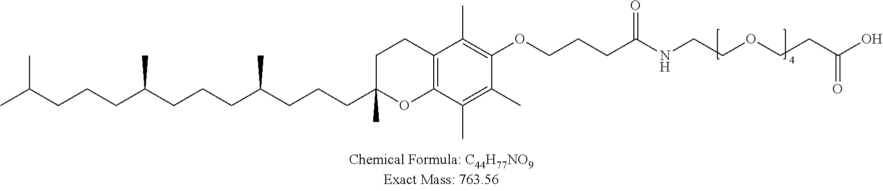

A solution of tert-butyl 17-oxo-20-({(2R)-2,5,7,8-tetramethyl-2-[(4R,8R)-4,8,12-trimethyltridecyl]-3,4-dihydro-2H-chromen-6-yl}oxy)-4,7,10,13-tetraoxa-16-azaicosan-1-oate (6.0 g, 7.4 mmol) in DCM (10 mL) was treated with TFA (35 mL) and stirred at rt for 1.5 h. The reaction mixture was concentrated in vacuo and lyophilized. The crude material was dissolved in ethyl acetate (EtOAc) (40 mL) then washed with water (4×20 mL). The organic layer was dried over sodium sulfate, filtered and concentrated in vacuo to give the title compound (5.9 g, >99%) as a yellow gum. The material was used in the next step without further purification.

¹H NMR (400 MHz, DMSO-d₆) δ 7.94 (t, 1H), 3.50-3.62 (m, 5H), 3.46-3.50 (m, 11H), 3.36-3.43 (m, 2H), 3.20 (q, 2H), 2.43 (t, 2H), 2.29 (t, 2H), 2.05 (s, 3H), 2.03 (s, 3H), 1.97 (s, 3H), 1.87-1.95 (m, 2H), 1.65-1.75 (m, 2H), 1.44-1.56 (m, 3H), 1.31-1.41 (m, 4H), 1.17-1.29 (m, 7H), 1.16 (s, 3H), 0.97-1.14 (m, 7H), 0.77-0.88 (m, 12H).

### Step (v): N-{75-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-75-oxo-3,6,9,12,15,18,21,24,27,30,33,36,39,42,45,48,51,54,57,60,63,66,69,72-tetracosaoxapentaheptacont-1-yl}-17-oxo-20-({(2R)-2,5,7,8-tetramethyl-2-[(4R,8R)-4,8,12-trimethyltridecyl]-3,4-dihydro-2H-chromen-6-yl}oxy)-4,7,10,13-tetraoxa-16-azaicosan-1-amide (Compound 2)

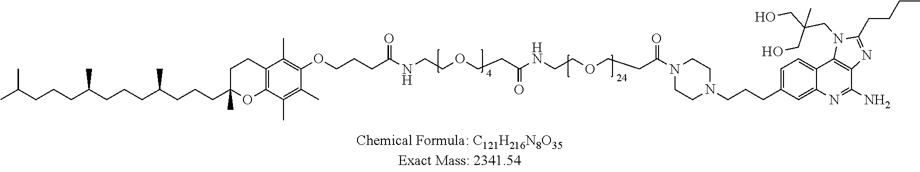

The reaction was conducted in two batches then combined for purification. In a round bottom flask, a solution of 17-oxo-20-({(2R)-2,5,7,8-tetramethyl-2-[(4R,8R)-4,8,12-trimethyltridecyl]-3,4-dihydro-2H-chromen-6-yl}oxy)-4,7,10,13-tetraoxa-16-azaicosan-1-oic acid (1.0 g, 1.3 mmol) in DMF (25 mL) was cooled to 0° C. and treated with HATU (498 mg, 1.31 mmol) followed by 1-amino-75-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-3,6,9,12,15,18,21,24,27,30,33,36,39,42,45,48,51,54,57,60,63,66,69,72-tetracosaoxapentaheptacontan-75-one (from Example 1, step xiii) (5.08 g, 1.31 mmol), DIPEA (3.72 g, 28.8 mmol) and additional DMF (25 mL). The reaction mixture was stirred at rt for 16 h. A second batch of the reaction was conducted with 1-amino-75-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-3,6,9,12,15,18,21,24,27,30,33,36,39,42,45,48,51,54,57,60,63,66,69,72-tetracosaoxapentaheptacontan-75-one (from Example 1, step xiii) (1.02 g, 0.262 mmol). The combined reaction was poured into water (200 mL) then extracted with DCM/MeOH (10:1, 3×300 mL). The combined organic layer was concentrated in vacuo and the residue was purified by prep HPLC (Boston Prime 150*30 mm*5 um, eluting with A: water (10 mM NH₄HCO₃), B: MeCN/THF (1:1); A %/B %: 53/47 to 70/30 over 21 min. Flow 30 mL/min, 17 injections) to give the title compound (1.2 g, 39%) as a white solid.

MS(ES+): 586.7 (M/4+H⁺), 781.9 (M/3+H⁺), 1172.3 (M/2+H⁺).

¹H NMR (400 MHz, DMSO-d₆) δ 8.41 (d, 1H), 7.92 (td, 2H), 7.39 (s, 1H), 7.05 (d, 1H), 6.35 (br s, 2H), 4.97 (t, 2H), 4.74 (br s, 1H), 4.43 (br s, 1H), 3.54-3.70 (m, 5H), 3.46-3.53 (m, 112H), 3.37-3.41 (m, 5H), 3.15-3.22 (m 5H), 2.87-3.10 (m, 2H), 2.68 (t, 2H), 2.52-2.58 (m, 2H), 2.25-2.35 (m, 10H), 2.06 (s, 3H), 2.03 (s, 3H), 1.97 (s, 3H), 1.88-1.94 (m, 2H), 1.66-1.85 (m, 6H), 1.30-1.55 (m, 9H), 1.17-1.29 (m, 7H), 1.16 (s, 3H), 0.97-1.14 (m, 7H), 0.93 (t, 3H), 0.78-0.87 (m, 12H), 0.54 (s, 3H).

The compounds in Table 1 were prepared using general methods or according/analogous to the methods of Schemes I-V and Examples 1-4, including modification, as appropriate.

TABLE 1

| Com- | | |
|---|---|---|
| pound | | Method/ |
| Num- | | Characterizing |
| ber | Structure/Compound Name | Data |
| 3 | 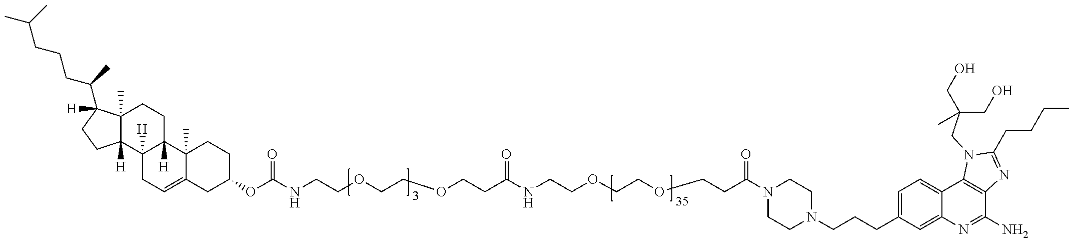 | Method as per Example 1 LC/MS Method A Retention time 1.24 min Observed mass 1393.6 (M/2 + 1) |
| | (3beta)-cholest-5-en-3-yl{127-[4-(3-{4-amino-2-butyl-1-[3-hydroxy-2- | |
| | (hydroxymethyl)-2-methylpropyl]-1H-imidazo[4,5-c]quinolin-7- | |
| | yl}propyl)piperazin-1-yl]-15,127-dioxo- | |
| | 3,6,9,12,19,22,25,28,31,34,37,40,43,46,49,52,55,58,61,64,67,70,73,76, | |
| | 79,82,85,88,91,94,97,100,103,106,109,112,115,118,121,124- | |
| | tetracontaoxa-16-azaheptacosahect-1-yl}carbamate | |
| 4 | 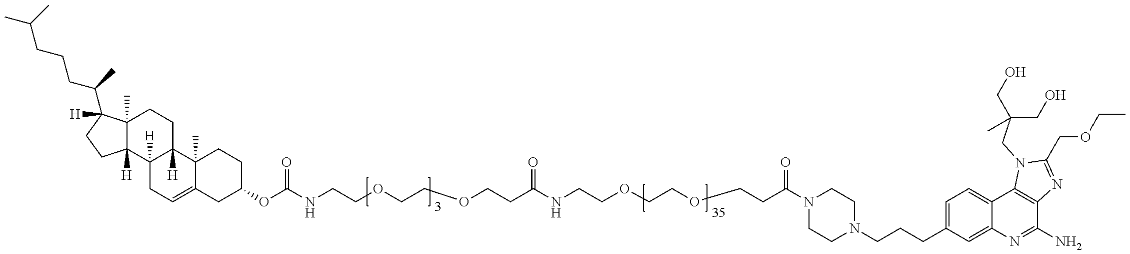 | Method as per Example 1 substitute 2- ethoxyacetyl chloride for pentanoyl chloride in Step (v) LC/MS Method A Retention time 1.24 min Observed mass 1394.1 (M/2 + 1) |
| | (3beta)-cholest-5-en-3-yl{127-[4-(3-{4-amino-2-(ethoxymethyl)-1-[3- | |
| | hydroxy-2-(hydroxymethyl)-2-methylpropyl]-1H-imidazo[4,5- | |
| | c]quinolin-7-yl}propyl)piperazin-1-yl]-15,127-dioxo- | |
| | 3,6,9,12,19,22,25,28,31,34,37,40,43,46,49,52,55,58,61,64,67,70,73,76, | |
| | 79,82,85,88,91,94,97,100,103,106,109,112,115,118,121,124- | |
| | tetracontaoxa-16-azaheptacosahect-1-yl}carbamate | |
| 5 | 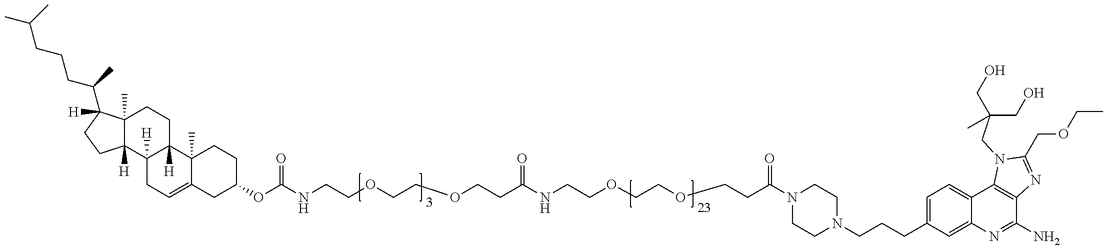 | Method as per Example 1 substitute 2- ethoxyacetyl chloride for pentanoyl chloride in Step (v) LC/MS Method A Retention time 1.23 min Observed Mass 1130.8 (M/2 + |
| | (3beta)-cholest-5-en-3-yl{91-[4-(3-{4-amino-2-(ethoxymethyl)-1-[3- | 1) |
| | hydroxy-2-(hydroxymethyl)-2-methylpropyl]-1H-imidazo[4,5- | |
| | c]quinolin-7-yl}propyl)piperazin-1-yl]-15,91-dioxo- | |
| | 3,6,9,12,19,22,25,28,31,34,37,40,43,46,49,52,55,58,61,64,67,70,73,76, | |
| | 79,82,85,88-octacosaoxa-16-azahennonacont-1-yl}carbamate | |
| 6 | 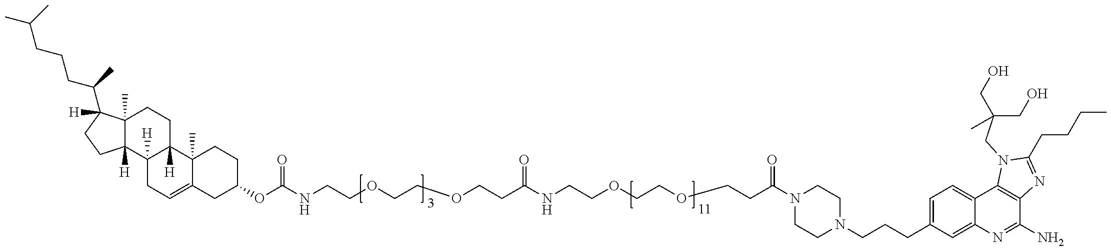 | Method as per Example 1 LC/MS Method A Retention time 1.23 min Observed mass 865.2 (M/2 + 1) |
| | (3beta,20R)-cholest-5-en-3-yl{5-[4-(3-{4-amino-2-butyl-1-[3- | |
| | hydroxy-2-(hydroxymethyl)-2-methylpropyl]-1H-imidazo[4,5- | |
| | c]quinolin-7-yl}propyl)piperazin-1-yl]-15,55-dioxo- | |
| | 3,6,9,12,19,22,25,28,31,34,37,40,43,46,49,52-hexadecaoxa-16- | |
| | azapentapentacont-1-yl}carbamate | |
| 7 | 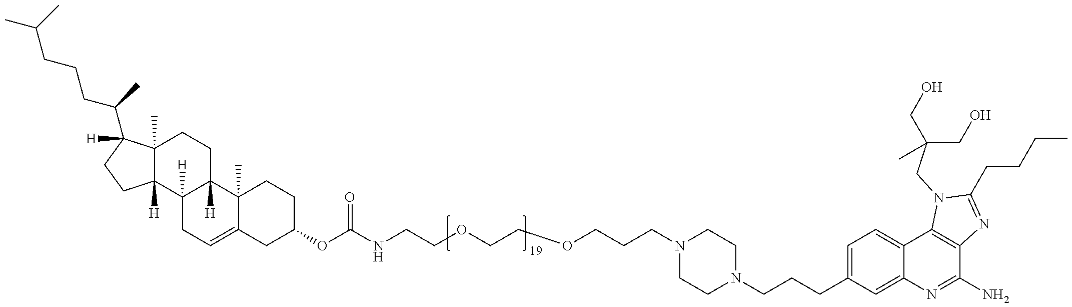 | See method described hereinbelow LC/MS Method A Retention time 1.13 min Observed mass 910,.7 (M/2 + 1) |
| | (3beta)-cholest-5-en-3-yl{63-[4-(3-{4-amino-2-butyl-1-[3-hydroxy-2- | |
| | (hydroxymethyl)-2-methylpropyl]-1H-imidazo[4,5-c]quinolin-7- | |
| | yl}propyl)piperazin-1-yl]- | |
| | 3,6,9,12,15,18,21,24,27,30,33,36,39,42,45,48,51,54,57,60- | |
| | icosaoxatrihexacont-1-yl}carbamate | |
| 8 | 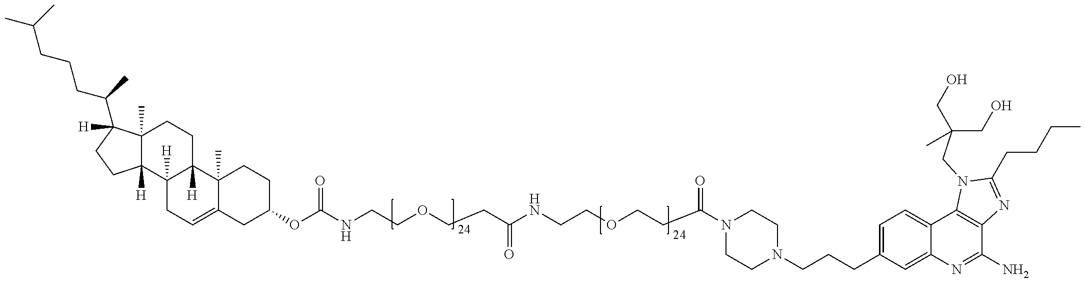 | Method as per Example 1 LC/MS Method B Retention time 1.11 min Observed mass 1046.9 (M/3 + 1) |
| | (3beta,20R)-cholest-5-en-3-yl{151-[4-(3-{4-amino-2-butyl-1-[3- | |
| | hydroxy-2-(hydroxymethyl)-2-methylpropyl]-1H-imidazo[4,5- | |
| | c]quinolin-7-yl}propyl)piperazin-1-yl]-75,151-dioxo- | |
| | 3,6,9,12,15,18,21,24,27,30,33,36,39,42,45,48,51,54,57,60,63,66,69.72, | |
| | 79,82,85,88,91,94,97,100,103,106,109,112,115,118,121,124,127,130, | |
| | 133,136,139,142,145,148-octatetracontaoxa-76- | |
| | azahenpentacontahect-1-yl}carbamate | |
| 9 | 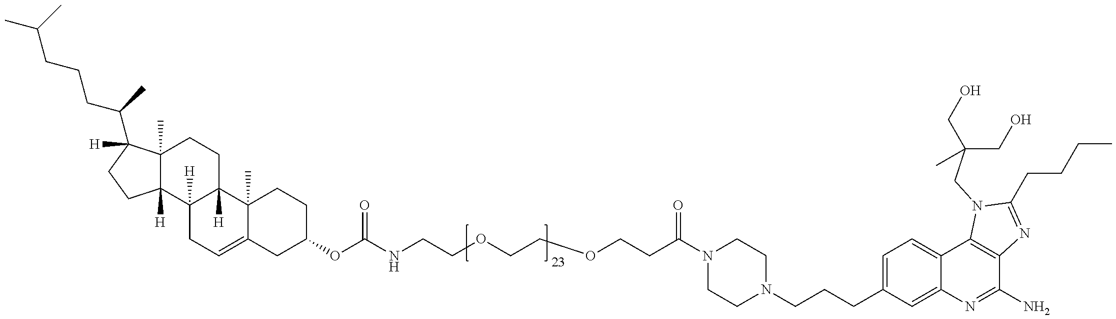 | Method as per Example 1 LC/MS Method A Retention time 1.1 min Observed mass 1005.6 (M/2 + 1) |
| | (3beta)-cholest-5-en-3-yl{75-[4-(3-{4-amino-2-butyl-1-[3-hydroxy-2- | |
| | (hydroxymethyl)-2-methylpropyl]-1H-imidazo[4,5-c]quinolin-7- | |
| | yl}propyl)piperazin-1-yl]-75-oxo- | |
| | 3,6,9,12,15,18,21,24,27,30,33,36,39,42, | |
| | 45,48,51,54,57,60,63,66,69,72-tetracosaoxapentaheptacont-1- | |
| | yl}carbamate | |
| 10 | 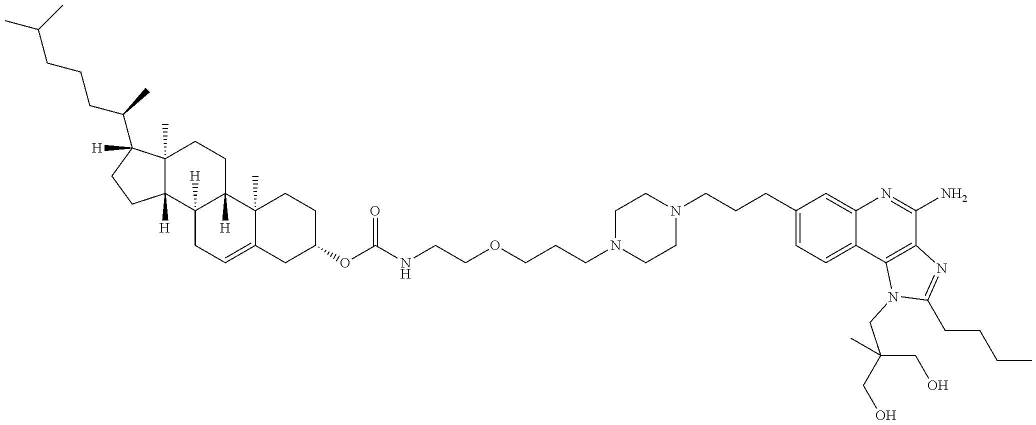 | Method as per Example 5 LC/MS Method A Retention time 1.1 min Observed mass 982.7 (M + 1) |
| | (3beta,20R)-cholest-5-en-3-yl(2-{3-[4-(3-{4-amino-2-butyl-1-[3- | |
| | hydroxy-2-(hydroxymethyl)-2-methylpropyl]-1H-imidazo[4,5- | |
| | c]quinolin-7-yl}propyl)piperazin-1-yl]propoxy}ethyl)carbamate | |
| 11 | 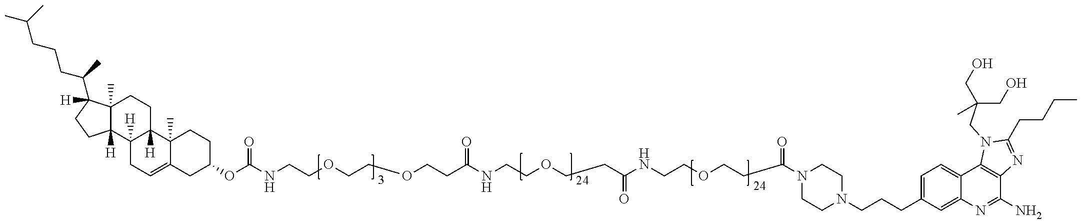 | Method as per Example 1 LC/MS Method B Retention time 1.11 min Observed mass 1129.6 (M/3 + 1) |
| | (3beta)-cholest-5-en-3-yl{167-[4-(3-{4-amino-2-butyl-1-[3-hydroxy-2- | |
| | (hydroxymethyl)-2-methylpropyl]-1H-imidazo[4,5-c]quinolin-7- | |
| | yl}propyl)piperazin-1-yl]-15,91,167-trioxo- | |
| | 3,6,9,12,19,22,25,28,31,34,37, | |
| | 40,43,46,49,52,55,58,61,64,67,70,73,76,79,82,85,88,95,98,101,104 | |
| | 107,110,113,116,119,122,125,128,131,134,137,140,143,146,149,152, | |
| | 155,158,161,164-dopentacontaoxa-16,92- | |
| | diazaheptahexacontahect-1-yl}carbamate | |
| 12 | 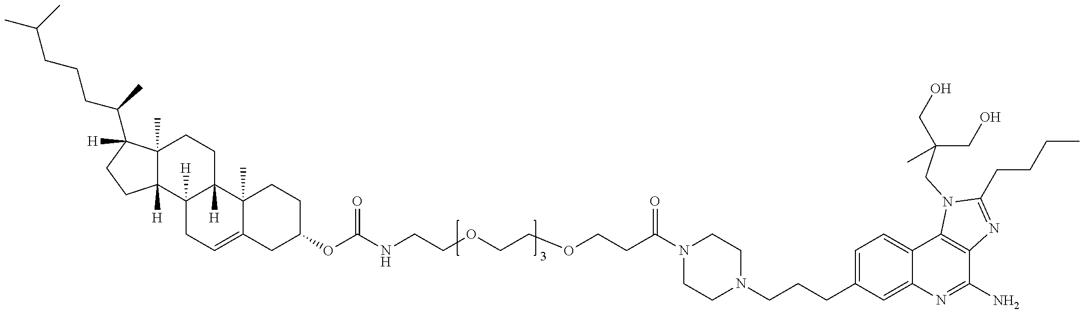 | Method as per Example 1 LC/MS Method A Retention time 1.12 min Observed mass 1128.9 (M + 1) |
| | (3beta,20R)-cholest-5-en-3-yl{15-[4-(3-{4-amino-2-butyl-1-[3- | |
| | hydroxy-2-(hydroxymethyl)-2-methylpropyl]-1H-imidazo[4,5- | |
| | c]quinolin-7-yl}propyl)piperazin-1-yl]-15-oxo-3,6,9,12- | |
| | tetraoxapentadec-1-yl}carba- | |
| | mate | |
| 13 | 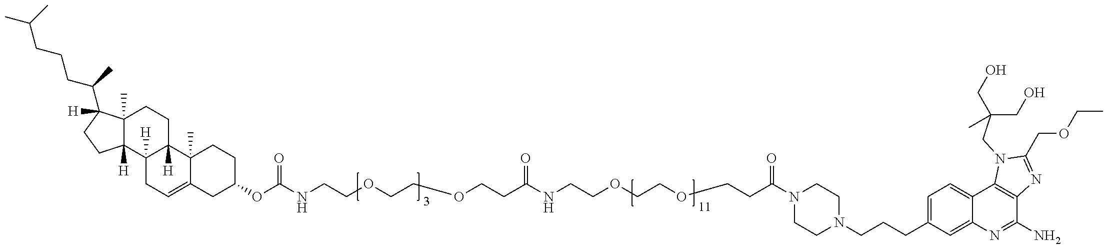 | Method as per Example 1 substitute 2- ethoxyacetyl chloride for pentanoyl chloride in Step (v) LC/MS Method A Retention time 1.24 min Observed mass 865.9 (M /2 + |
| | (3beta,20R)-cholest-5-en-3-yl{55-[4-(3-{4-amino-2-(ethoxymethyl)-1- | 1) |
| | [3-hydroxy-2-(hydroxymethyl)-2-methylpropyl]-1H-imidazo[4,5- | |
| | c]quinolin-7-yl}propyl)piperazin-1-yl]-15,55-dioxo- | |
| | 3,6,9,12,19,22,25,28, | |
| | 31,34,37,40,43,46,49,52-hexadecaoxa-16-azapentapentacont-1- | |
| | yl}carbamate | |
| 14 | 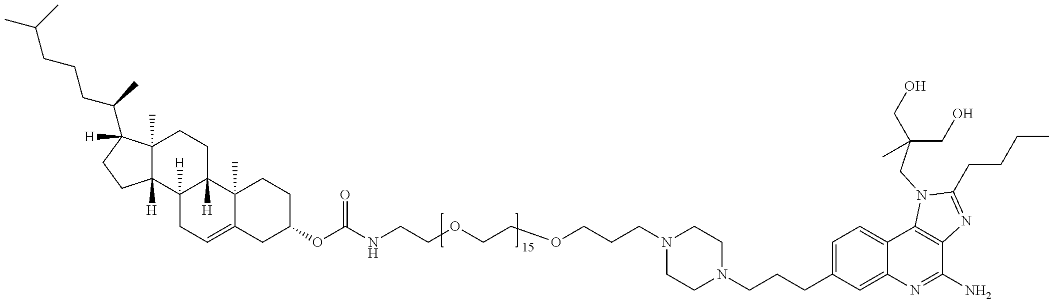 | Method as per Example 5 LC/MS Method A Retention time 1.14 min Observed mass 822.6 (M/2 + 1) |
| | (3beta,20R)-cholest-5-en-3-yl{51-[4-(3-{4-amino-2-butyl-1-[3- | |
| | hydroxy-2-(hydroxymethyl)-2-methylpropyl]-1H-imidazo[4,5- | |
| | c]quinolin-7-yl}propyl)piperazin-1-yl]- | |
| | 3,6,9,12,15,18,21,24,27,30,33,36,39,42,45, | |
| | 48-hexadecaoxahenpentacont-1-yl}carbamate | |
| 15 | 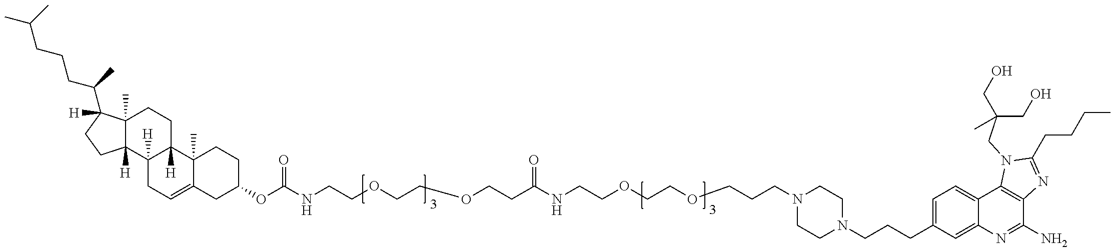 | Method as per Example 5 LC/MS Method A Retention time 1.11 min Observed mass 681.8 (M/2 + 1) |
| | (20R)-cholest-5-en-3beta-yl{31-[4-(3-{4-amino-2-butyl-1-[3-hydroxy- | |
| | 2-(hydroxymethyl)-2-methylpropyl]-1H-imidazo[4,5-c]quinolin-7- | |
| | yl}propyl)piperazin-1-yl]-15-oxo-3,6,9,12,19,22,25,28-octaoxa-16- | |
| | azahentriacontan-1-yl}carbamate | |
| 16 | 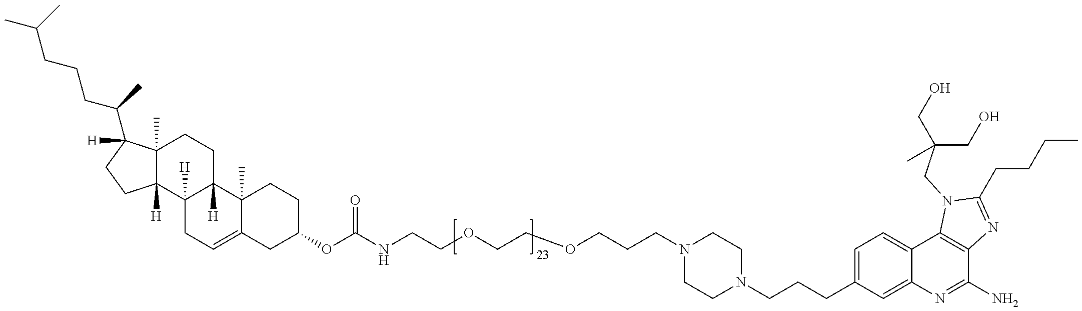 | Method as per Example 5 LC/MS Method A Retention time 1.16 min Observed mass 998.7 (M/2 + 1) |
| | (20R)-cholest-5-en-3beta-yl{75-[4-(3-{4-amino-2-butyl-1-[3-hydroxy- | |
| | 2-(hydroxymethyl)-2-methylpropyl]-1H-imidazo[4,5-c]quinolin-7- | |
| | yl}propyl)piperazin-1-yl]- | |
| | 3,6,9,12,15,18,21,24,27,30,33,36,39,42,45,48 | |
| | 51,54,57,60,63,66,69,72-tetracosaoxapentaheptacontan-1- | |
| | yl}carbamate | |
| 17 | 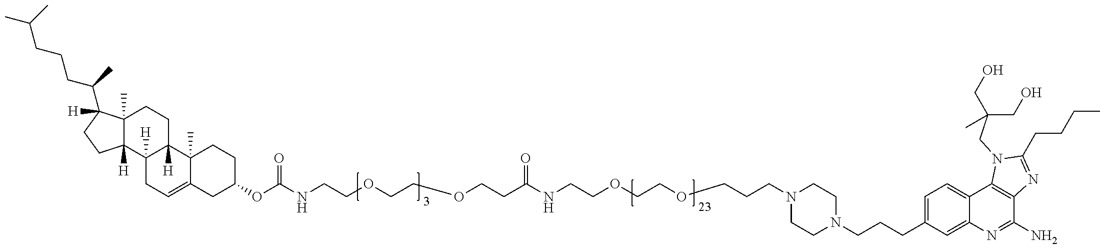 | Method as per Example 5 LC/MS Method A Retention time 1.09 min Observed mass 1122.2 (M/2 + 1) |
| | (3beta)-cholest-5-en-3-yl{91-[4-(3-{4-amino-2-butyl-1-[3-hydroxy-2- | |
| | (hydroxymethyl)-2-methylpropyl]-1H-imidazo[4,5-c]quinolin-7- | |
| | yl}propyl)piperazin-1-yl]-15-oxo- | |
| | 3,6,9,12,19,22,25,28,31,34,37,40,43,46, | |
| | 49,52,55,58,61,64,67,70,73,76,79,82,85,88-octacosaoxa-16- | |
| | azahennonacont-1-yl}carbamate | |
| 18 | 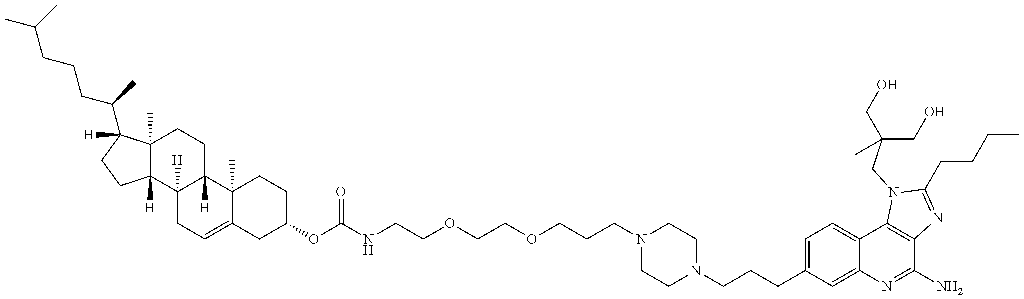 | Method as per Example 5 LC/MS Method A Retention time 1.16 min Observed mass 1026.8 (M + 1) |
| | (20R)-cholest-5-en-3beta-yl[2-(2-{3-[4-(3-{4-amino-2-butyl-1-[3- | |
| | hydroxy-2-(hydroxymethyl)-2-methylpropyl]-1H-imidazo[4,5- | |
| | c]quinolin-7-yl}propyl)piperazin-1-yl]propoxy}ethoxy)ethyl]carbamate | |
| 19 | 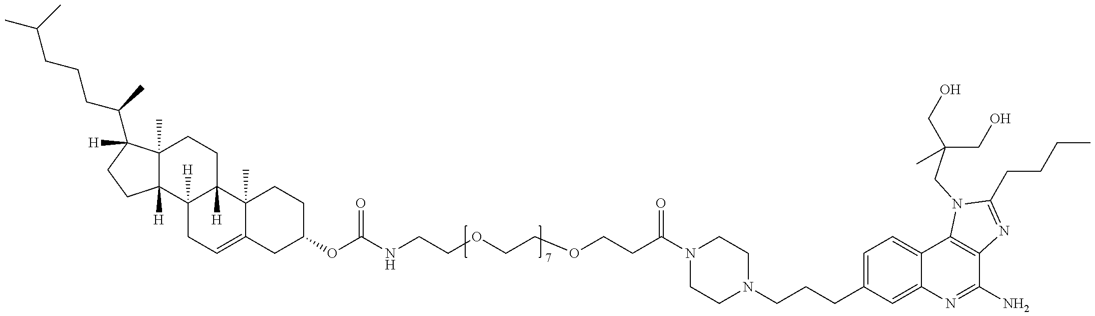 | Method as per Example 1 LC/MS Method A Retention time 1.11 min Observed mass 1304.8 (M + 10 |
| | (20R)-cholest-5-en-3beta-yl{27-[4-(3-{4-amino-2-butyl-1-[3-hydroxy- | |
| | 2-(hydroxymethyl)-2-methylpropyl]-1H-imidazo[4,5-c]quinolin-7- | |
| | yl}propyl)piperazin-1-yl]-27-oxo-3,6,9,12,14,18,21,24- | |
| | octaoxaheptacosan-1-yl}carbamate | |
| 20 | 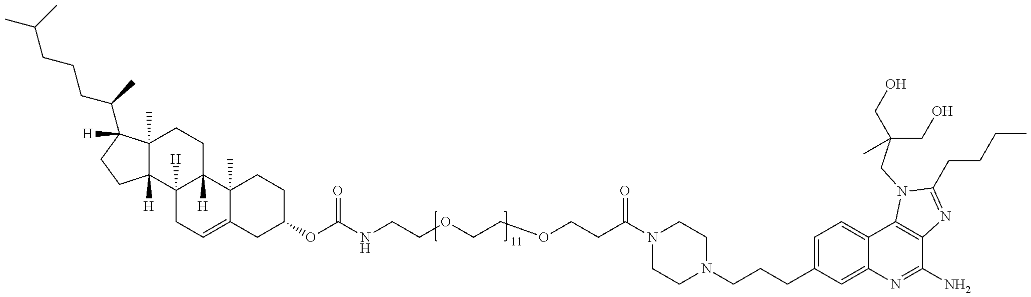 | Method as per Example 1 LC/MS Method B Retention time 1.10 min Observed mass 741.6 (M/2 + 1) |
| | (20R)-cholest-5-en-3beta-yl{39-[4-(3-{4-amino-2-butyl-1-[3-hydroxy- | |
| | 2-(hydroxymethyl)-2-methylpropyl]-1H-imidazo[4,5-c]quinolin-7- | |
| | yl}propyl)piperazin-1-yl]-39-oxo-3,6,9,12,15,18,21,24,27,30,33,36- | |
| | dode- | |
| | caoxanonatriacontan-1-yl}carbamate | |
| 21 | 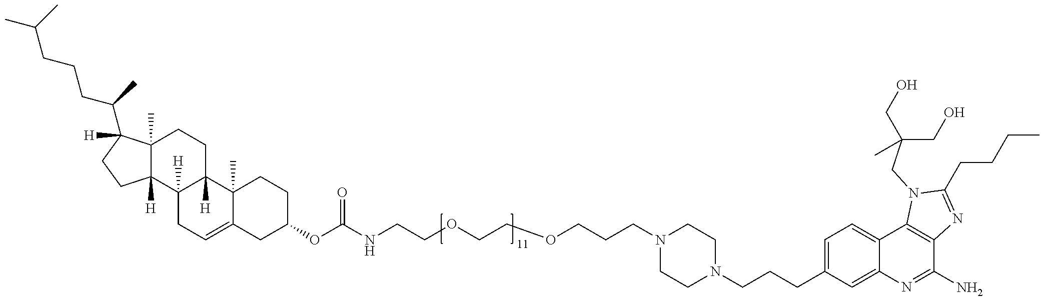 | Method as per Example 5 LC/MS Method A Retention time 1.10 min Observed mass 146..9 (M + 1) |
| | (20R)-cholest-5-en-3beta-yl{39-[4-(3-{4-amino-2-butyl-1-[3-hydroxy- | |
| | 2-(hydroymethyl)-2-methylpropyl]-1H-imidazo[4,5-c]quinolin-7- | |
| | yl}propyl)piperazin-1-yl]-3,6,9,12,15,18,21,24,27,30,33,36- | |
| | dodecaoxano- | |
| | natriacontan-1-yl}carbamate | |
| 22 | 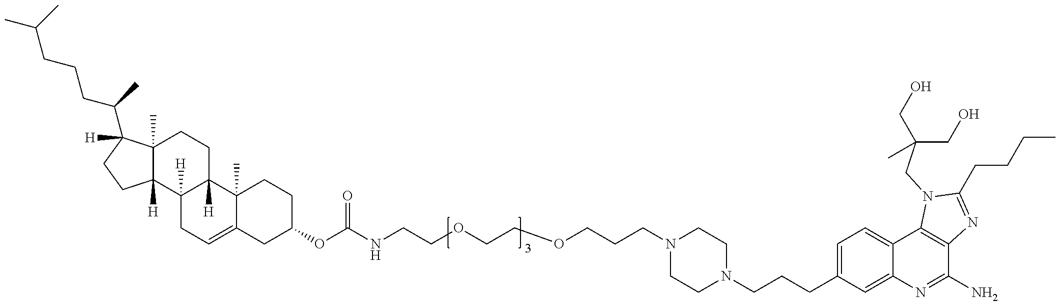 | Method as per Example 5 LC/MS Method B Retention time 1.13 min Observed mass 1115.0 (M + 1) |
| | (20R)-cholest-5-en-3beta-yl{15-[4-(3-{4-amino-2-butyl-1-[3-hydroxy- | |
| | 2-(hydroxymethyl)-2-methylpropyl]-1H-imidazo[4,5-c]quinolin-7- | |
| | yl}propyl)piperazin-1-yl]-3,6,9,12-tetraoxapentadecan-1-yl}carbamate | |
| 23 | 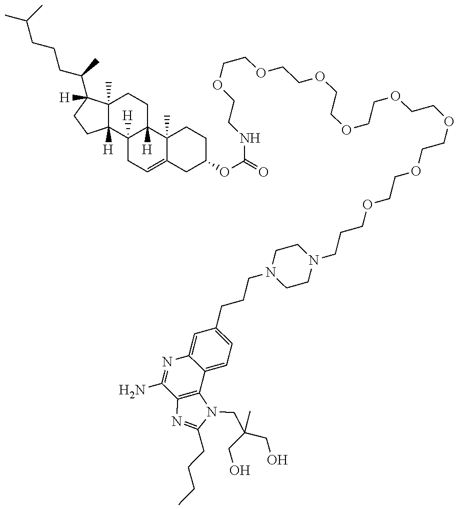 | Method as per Example 5 LC/MS Method B Retention time 1.13 min Observed mass 646.4 (M/2 + 1) |
| | (20R)-cholest-5-en-3beta-yl{27-[4-(3-{4-amino-2-butyl-1-[3-hydroxy- | |
| | 2-(hydroxymethyl)-2-methylpropyl]-1H-imidazo[4,5-c]quinolin-7- | |
| | yl}propyl)piperazin-1-yl]-3,69,12,15,18,21,24-octaoxaheptacosan-1- | |
| | yl}carbamate | |
| 24 | 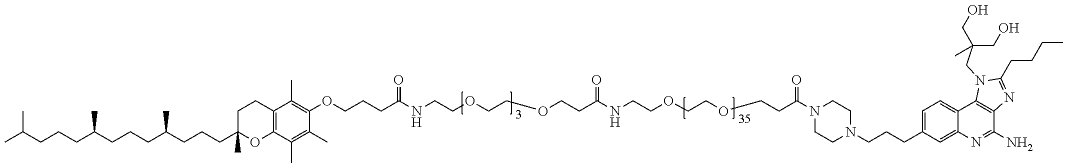 | Method as per Example 4 LC/MS Method B Retention time 1.13 min Observed mass 1459.5 [(M + 1) + 23] |
| | N-{111-[4-(3-{4-amino-2-butyl-1-[3-hydroxy-2-(hydroxymethyl)-2- | |
| | methylpropyl]-1H-imidazo[4,5-c]quinolin-7-yl}propyl)piperazin-1-yl]- | |
| | 111-oxo- | |
| | 3,6,9,12,15,18,21,24,27,30,33,36,39,42,45,48,51,54,57,60,63,66, | |
| | 69,72,75,78,81,84,87,90,93,96,99,102,105,108- | |
| | hexatriacontaoxahendecahectan-1-yl}-17-oxo-20-({(2R)-2,5,7,8- | |
| | tetramethyl-2-[(4R,8R)-4,8,12-trimethyltridecyl]-3,4-dihydro-2H-1- | |
| | benzopyran-6-yl}oxy)-4,7,1, | |
| | 0,13-tetraoxa-16-azaicosan-1-amide | |
| 25 | 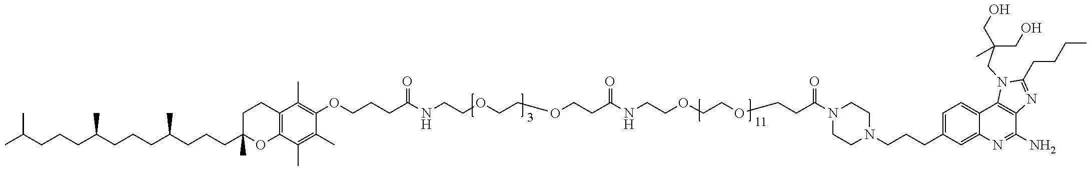 | Method as per Example 4 LC/MS Method A Retention time 1.17 min Observed mass 908.6 (M/2 + 1) |
| | N-{39-[4-(3-{4-amino-2-butyl-1-[3-hydroxy-2-(hydroxymethyl)-2- | |
| | methylpropyl]-1H-imidazo[4,5-c]quinolin-7-yl}propyl)piperazin-1-yl]- | |
| | 39-oxo-3,6,9,12,15,18,21,24,27,30,33,36-dodecaoxanonatriacontan- | |
| | 1-yl}- | |
| | 17-oxo-20-({(2R)-2,5,7,8-tetramethyl-2-[(4R,8R)-4,8,12- | |
| | trimethyltridecyl]-3,4-dihydro-2H-1-benzopyran-6-yl}oxy)-4,,10,13- | |
| | tetraoxa-16-azaicosan-1-amide | |
| 26 | 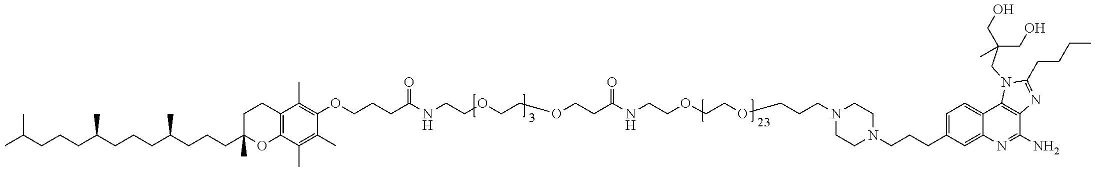 | Method as per Example 4 LC/MS Method A Retention time 1.17 min Observed mass 1165.2 (M/2 + 1) |
| | N-{75-[4-(3-{4-amino-2-butyl-1-[3-hydroxy-2-(hydroxymethyl)-2- | |
| | methylpropyl]-1H-imidazo[4,5-c]quinolin-7-yl}propyl)piperazin-1-yl]- | |
| | 3,6,9,12,15,18,21,24,27,30,33,36,39,42,45,48,51,54,57,60,63,66,69,72- | |
| | tetracosaoxapentaheptacontan-1-yl}-17-oxo-20-({(2R)-2,5,7,8- | |
| | tetramethyl-2-[(4R,8R)-4,8,12-trimethyltridecyl]-3,4-dihydro-2H-1- | |
| | benzopyran-6-yl}oxy)-4,7,10,13-tetraoxa-16-azaicosan-1-amide | |
| 27 | 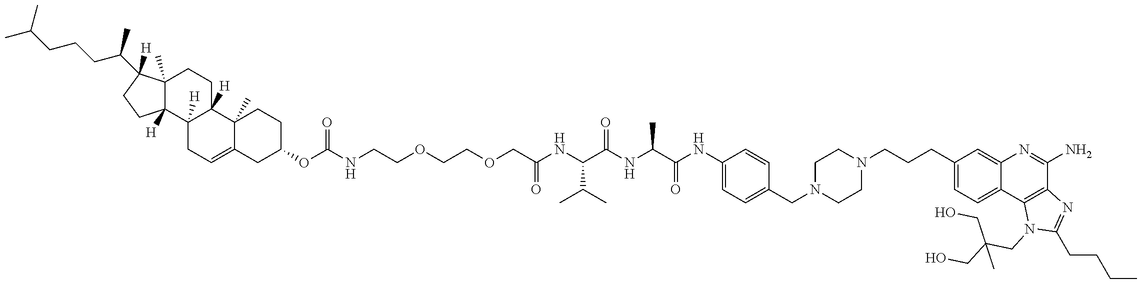 | Method as per Example 2 LC/MS Method A Retention time 1.125 min Observed mass 1302.0 (M + 1) |
| | (3beta)-cholest-5-en-3-yl{(2S,5S)-1-((4-((4-(3-(4-amino-2-butyl-1-(3- | |
| | hydroxy-2-(hydroxymethyl)-2-methylpropyl)-1H-imidazo[4,5- | |
| | c]quinolin-7-yl)propyl)piperazin-1-yl)methyl)phenyl)amino)-5- | |
| | isopropyl-2-methyl-1,4,7-trioxo-9,12-dioxa-3,6-diazatetradecan-14- | |
| | yl}carbamate | |
| 28 | 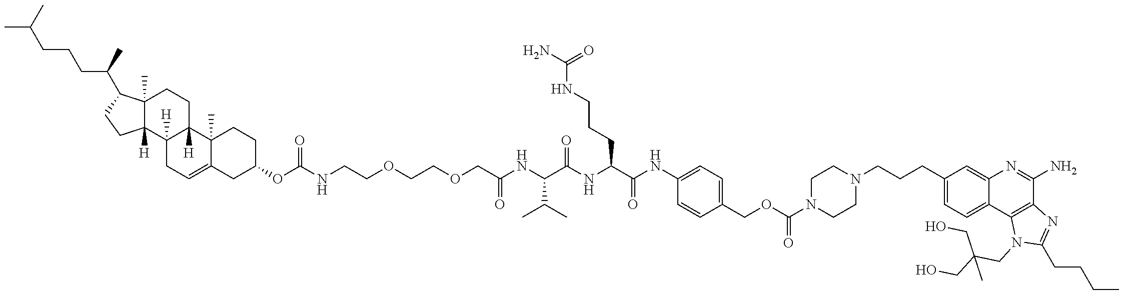 | Method as per Example 3 LC/MS Method A Retention time 1.100 min Observed mass 1432.6 (M + 1) |
| | 4-((12S,15S)-1-(((3beta)-cholest-5-en-3-yl)oxy)-12-isopropyl-1,10,13- | |
| | trioxo-15-(3-ureidopropyl)-5,8-dioxa-2,11,14-triazahexadecan-16- | |
| | amido)benzyl 4-(3-(4-amino-2-butyl-1-(3-hydroxy-2-(hydroxymethyl)- | |
| | 2-methylpropyl)-1H-imidazo[4,5-c]quinolin-7-yl)propyl)piperazine-1- | |
| | carboxylate | |
| 29 | 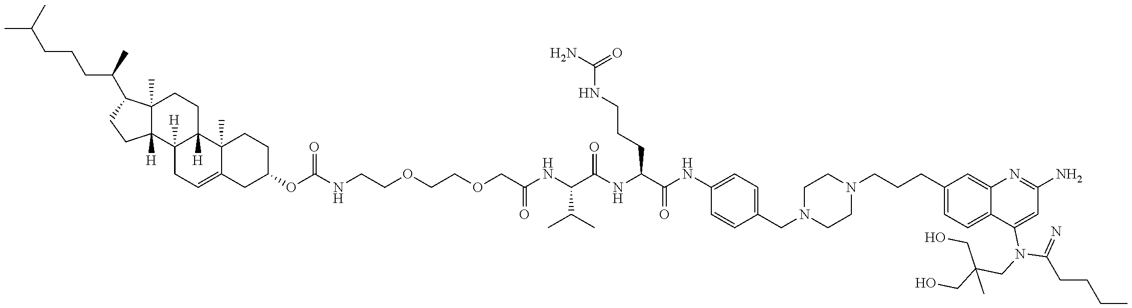 | Method as per Example 2 LC/MS Method A Retention time 1.100 min Observed mass 1388.1 (M + 1) |
| | (3beta)-cholest-5-en-3-yl((6S,9S)-1-amino-6-((4-((4-(3-(4-amino-2- | |
| | butyl-1-(3-hydroxy-2-(hydroxymethyl)-2-methylpropyl)-1H- | |
| | imidazo[4,5-c]quinolin-7-yl)propyl)piperazin-1- | |
| | yl)methyl)phenyl)carbamoyl)-9-isopropyl-1,8,11-trioxo-13,16-dioxa- | |
| | 2,7,10-triazaoctadecan-18-yl)carbamate | |

### Example 5

### Preparation of a piperazine derivative through reductive amination (3beta)-cholest-5-en-3-yl {63-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-3,6,9,12,15,18,21,24,27,30,33,36,39,42,45,48,5 1,54,57,60-icosaoxatrihexacont-1-yl}carbamate (Compound 7)

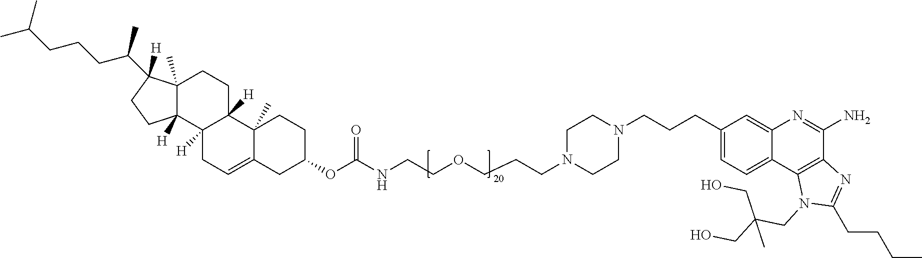

### Step (i): (3β)-cholest-5-en-3-yl (63-oxo-3,6,9,12,15,18,21,24,27,30,33,36,39,42,45,48,51,54,57,60-icosaoxatrihexacont-1-yl)carbamate

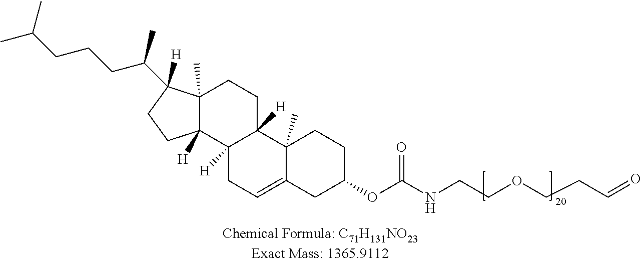

A solution of (3p)-cholest-5-en-3-yl (63-hydroxy-3,6,9,12,15,18,21,24,27,30,33,36,39,42,45,48,51,54,57,60-icosaoxatrihexacont-1-yl)carbamate (210 mg, 0.153 mmol) (made similarly to Example 1, Step ii) and Dess-Martin Periodinane (84.6 mg, 0.199 mmol) in DCM (5 mL) was stirred at rt for 2 h. The reaction mixture was filtered through paper and concentrated in vacuo. The crude product was purified using column chromatography eluting with DCM/MeOH (100:0 to 85:15) to give the title compound (180 mg, 86%) as a white gum.

¹H NMR (400 MHz, CDCl₃) δ 9.81 (s, 1H), 5.35-5.39 (m, 1H), 5.16-5.21 (m, 1H), 4.45-4.55 (m, 1H), 3.83 (t, 2H), 3.58-3.72 (m, 78H), 3.53-3.57 (m, 2H), 3.49 (s, 1H), 3.32-3.40 (m, 2H), 2.69 (td, 2H), 2.32-2.40 (m, 1H), 2.21-2.32 (m, 1H), 1.67-2.06 (m, 8H), 1.02-1.63 (m, 21H), 0.95-1.03 (m, 6H), 0.92 (d, 3H), 0.86 (d, 6H), 0.68 (s, 3H).

### Step (ii):(3β)-cholest-5-en-3-yl {63-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-3,6,9,12,15,18,21,24,27,30,33,36,39,42,45,48,51,54,57,60-icosaoxatrihexacont-1-yl}carbamate (Compound 7)

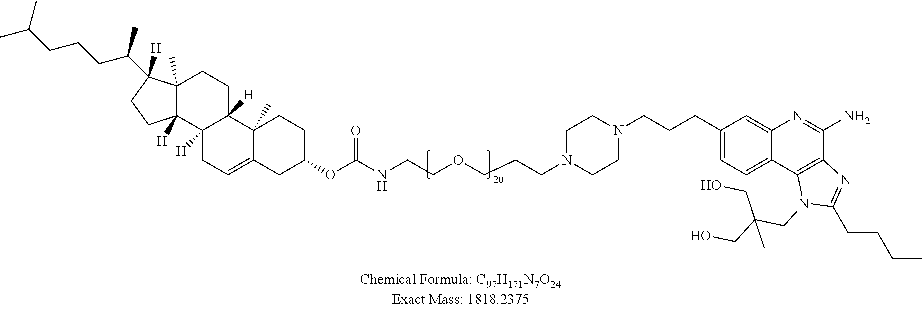

A mixture of 2-({4-amino-2-butyl-7-[3-(piperazin-1-yl)propyl]-1H-imidazo[4,5-c]quinolin-1-yl}methyl)-2-methylpropane-1,3-diol (60.9 mg, 0.105 mmol) and potassium acetate (31.0 mg, 0.316 mmol) in methanol (MeOH) (4 mL) was stirred at rt for 30 min. The reaction mixture was then treated with (3p)-cholest-5-en-3-yl (63-oxo-3,6,9,12,15,18,21,24,27,30,33,36,39,42,45,48,51,54,57,60-icosaoxatrihexacont-1-yl)carbamate (180 mg, 0.11 mmol) and sodium cyanoborohydride (26.5 mg, 0.421 mmol) and the resulting mixture was stirred at rt for 72 h. The reaction mixture was quenched with cold (0° C.) water (1 mL) and concentrated in vacuo. The crude product was purified using prep HPLC (Boston Prime C18 150*30 mm*5 μm, eluting with A: water (10 mM NH₄HCO₃)/B: acetonitrile (MeCN)/THF (1:1), A/B: 52:48 to 27:73 over 8 min, 30 mL/min and lyophilized to give the title compound (83.6 mg, 44%) as a yellow gum.

### MS(ES+): 910.7 (M/2+H⁺) (Method A)

¹H NMR (400 MHz, CD₃OD) δ 8.54 (d, 1H), 7.52 (s, 1H), 7.21 (d, 1H), 5.36 (br. s., 1H), 4.54-4.66 (m, 1H), 4.33-4.43 (m, 1H), 3.59-3.69 (m, 68H), 3.55-3.59 (m, 4H), 3.48-3.52 (m, 6H), 3.39-3.48 (m, 2H), 3.30-3.33 (m, 2H), 3.28 (t, 2H), 3.00-3.23 (m, 2H), 2.78 (t, 2H), 2.22-2.68 (m, 12H), 1.73-2.05 (m, 11H), 1.32-1.63 (m, 12H), 0.97-1.30 (m, 16H), 0.94 (d, 3H), 0.88 (d, 6H), 0.70 (s, 3H), 0.66 (s, 3H).

### Biological Testing

### Example 6: hTLR7 and hTLR8 In-Vitro Functional Assays

To determine the ability of test compounds to activate the human toll like receptor 7 (hTLR7) or human toll like receptor 8 (hTLR8), cell-based reporter systems were utilized. HEK293 cells stably overexpressing either hTLR7 or hTLR8 along with a reporter gene containing an optimized secreted embryonic alkaline phosphatase gene (SEAP), under the control of the IFN-b minimal promoter fused to five NF-kB and AP-1-binding sites, were obtained from Invivogen (HEK-Blue™ hTLR7, cat #Hkb-htlr7; HEK-Blue™ hTLR8, cat #Hkb-htlr8). Stimulation of hTLR7 or hTLR8 in these cells activates NF-kB and AP-1 and induces the production of SEAP which can be quantified using an alkaline phosphatase detection reagent.

Cells were maintained in DMEM growth media containing heat inactivated (10%), Glutamax (2 mM), Penicillin/Streptomycin, Blasticidin (10 ug/ml), Zeocin (100 ug/ml) and Normocin (100 ug/ml) according to the manufacturer suggestion. On day one of the assay, compounds were prepared using 11-point half-log serial dilutions from a 10 mM DMSO stock solution and 50 nL was spotted into 384-well plates (PerkinElmer, cat #6007480). Positive and negative controls were also spotted within the assay plate and were used to determine percent effect during the analysis process. After resuspension in DMEM assay media containing FBS heat inactivated (10%), Glutamax (2 mM) and Penicillin/Streptomycin, 10,000 cells/20 ul/well were added to previously prepared compound plates. Plates were incubated overnight (16-20 hrs) at 37° C. in a 5% CO₂ environment. Prewetted Microclime lids (Labcyte, LLS-0310) were used to prevent evaporation. On day two of the assay, QUANTI-Blue™ detection reagent was prepared by reconstituting QUANTI-Blue™ powder (InvivoGen, Rep-qb1) with 100 ml of sterile water and allowed to equilibrate to 37° C. for 15 minutes. 20 ul of QUANTI-Blue™ detection reagent was added to each well and plates were incubated at room temperature for 180 min. At the end of the incubation, plates were read on an Envision (Perkin Elmer) plate reader capturing absorbance at 650 nm.

Using Positive (tool compound) and Negative (DMSO) controls, the percent (%) effect was calculated for each sample using the following equation:

$$\% effect = 100 - 100 * ( ( Sample - Positive ) / ( Negative - Positive ) )$$

The % effect at each concentration of compound was calculated utilizing the ABase software suite (IBDS) and was relative to the amount of SEAP produced in the positive and negative control wells contained within each assay plate. The concentrations and % effect values for test compounds were fit using a 4-parameter logistic model in ABase and the concentration of compound that produced 50% response (EC₅₀) was calculated.

TABLE 2

| Compound | hTLR7 HEK Blue | hTLR8 HEK Blue |
|---|---|---|
| Number | EC50 (uM) | EC50 (uM) |
| 1 | 0.122 | 1.363 |
| 2 | 0.355 | 5.033 |
| 3 | 0.053 | 0.339 |
| 4 | 0.060 | 1.745 |
| 5 | 0.072 | 2.304 |
| 6 | 0.078 | 5.075 |
| 7 | 0.140 | 14.395 |
| 8 | 0.150 | 0.731 |
| 9 | 0.181 | 15.970 |
| 10 | 0.186 | 9.907 |
| 11 | 0.191 | 0.583 |
| 12 | 0.210 | 19.631 |
| 13 | 0.223 | 3.326 |
| 14 | 0.236 | 22.579 |
| 15 | 0.276 | 17.570 |
| 16 | 0.278 | >25.000 |
| 17 | 0.282 | 2.613 |
| 18 | 0.288 | 9.896 |
| 19 | 0.362 | 20.243 |
| 20 | 0.443 | 21.772 |
| 21 | 0.533 | >25.000 |
| 22 | 1.155 | 11.711 |
| 23 | 1.676 | 14.924 |
| 24 | 0.134 | 2.377 |
| 25 | 0.312 | 8.280 |
| 26 | 0.404 | >25.000 |
| 27 | 3.095 | 2.971 |
| 28 | 1.887 | 4.483 |
| 29 | 0.527 | 5.026 |

### Example 7: Lymph Node Localization of AMP Adjuvants in Female BALB/c Mice

Test compounds were prepared at a concentration of 40 uM in phosphate-buffered saline (pH 7.4)+0.1% polysorbate 80, and 50 uL (2 nmol) of each was administered by intramuscular injection into the right hind leg muscle (gastrocnemius) of female BALB/c mice. Lymph nodes (popliteal, inguinal) were collected from 5 mice per group at 4 h and 48 h post injection, snap frozen in liquid nitrogen, and stored at −80C until analysis. Blood was also collected via cardiac puncture at 4 h and 48 h post injection and centrifuged in EDTA-coated tubes to yield plasma which was stored at −80C until analysis. AMP adjuvant concentrations in each sample were quantified by liquid chromatography-mass spectrometry (LC-MS).

A standard curve containing the test compound was prepared in control mouse plasma before extraction with protein precipitation. Muscle samples were weighed and diluted in 4× 60:40 Isopropanol (IPA):Water. Lymph node samples were assumed to be 1 mg and were diluted 50× in 49 mL 60:40 IPA:Water. Zirconium beads were added to tissue samples, followed by shaking in a bead beater to yield tissue homogenate. For plasma analysis, 20 ml of test sample or standard was added followed by 120 ml of acetonitrile containing internal standard (Simvastatin at 300 ng/ml). Tissue homogenate was prepared for analysis using a mixed matrix approach where test tissue samples (20 ml) were matrix matched with control plasma, and a plasma standard curve was matrixed matched with control mouse tissue. Internal standard was added as described above. Samples were vortexed for 1 min, then centrifuged at 3000 rpm for 5 min. 50 ml of supernatant was removed and transferred to a clean 96 well block, 150 ml of 0.1% formic acid in water was added, block was vortexed, centrifuged and 15 ml was injected on the LC/MS for analysis.

Separation was achieved with a Waters Acquity UPLC HSS Column (2.1*50 mm, 1.8 mm), and a gradient of 0.1% formic acid in water (Mobile Phase A) and 0.1% formic acid in acetonitrile (Mobile Phase B) at a flow rate of 0.6 mL/min. An initial mobile phase composition of 5% B was ramped to 95% in 2.0 minutes, held at 95% for 0.3 minutes, and then returned to initial 5% B for 0.3 minutes re-equilibration. The total analysis time for each sample was 3 minutes.

Data was collected on an AB Sciex API6500 (QTRAP) mass spectrometer using positive Turbo IonSpray™ electrospray ionization (ESI) and multiple reaction monitoring (MRM) mode. Typical source conditions, heated capillary temperature, gas1, gas2, and curtain gas were set at 600C, 50, 50, and 10 respectively. Transitions for Compound 1, Compound 2, and internal standard Simvastatin were m/z 1129® 923 for Compound 1, m/z 1172® 956 for Compound 2 and m/z 419® 285 for internal standard Simvastatin respectively. Data acquisition and processing was carried out with Analyst software version 1.7.

### Results:

### Compound 1

The concentration of Compound 1 in the popliteal lymph node was 45700±30700 ng/g and 27600±12600 ng/g at 4 h and 48 h post injection, respectively. The concentration of Compound 1 in the inguinal lymph node was 18300±3160 ng/g and 17300±7400 ng/g at 4 h and 48 h post injection, respectively. The concentration of Compound 1 in plasma was 268±59.6 ng/mL and 12.5±3.82 ng/mL at 4 h and 48 h post injection, respectively.

### Compound 2

The concentration of Compound 2 in the popliteal lymph node was 35300±16800 ng/g and 23200±7820 ng/g at 4 h and 48 h post injection, respectively. The concentration of Compound 2 in the inguinal lymph node was 16600±6920 ng/g and 17000±3320 ng/g at 4 h and 48 h post injection, respectively. The concentration of Compound 2 in plasma was 1100±305 ng/mL and BLQ (<25 ng/mL) at 4 h and 48 h post injection, respectively.

### In-vivo Immunogenicity

### Example 8

In two independent in vivo immunogenicity studies, female BALB/c mice were immunized intramuscularly twice three weeks apart (days 0 and 21) with 0.5 μg of RSV antigen alone or mixed with 2 nmol of Example 1 adjuvant (Compound 1). Sera were collected at day 35 to determine RSV neutralization antibody titers (nAb). The RSV antigen tested was bivalent comprising a RSV F Protein of Subtype A comprising the sequence of SEQ ID NO: 1 and SEQ ID NO: 2, as well as a RSV F Protein of Subtype B comprising the sequence of SEQ ID NO: 3 and SEQ ID NO: 4.

TABLE 3

| Study 1 | RSV Antigen alone | RSV Antigen/ Compound 1 |
|---|---|---|
| GMT | 466 | 7502 |
| Study 2 | RSV Antigen alone | RSV Antigen/ Compound 1 |
| GMT | 1962 | 5362 |

50% neutralizing antibody titers (nAb) to RSV (geometric mean titer, GMT) are shown in Table 3. 50% nAb responses against RSV A strain (M37) were measured by an RSV microneutralization assay at 2 weeks post dose 2.

### Example 9

In this study, the immunogenicity of adjuvanted Streptococcus pneumoniae (S. pneumoniae) serotype 3 was evaluated in a mouse model. As shown in Table 4 below, the adjuvants evaluated included aluminum phosphate (AlPO₄), the adjuvant of Example 1 (Compound 1), the adjuvant of Example 4 (Compound 2), and a free TLR 7/8 agonist molecule (a molecule comprising the TLR 7/8 agonist moiety of Compounds 1 and 2 but without an attached cholesterol or tocopherol group). The antigen tested was a Streptococcus pneumoniae serotype 3 polysaccharide conjugated to a CRM₁₉₇ carrier.

TABLE 4
Study Design of Example 9

| | | | | | Vaccine | Assay |
|---|---|---|---|---|---|---|
| | | | | | time | time |
| | Antigen | | Adjuvant | | points | point |
| Antigen | dose | Adjuvant | dose | | (weeks) | (weeks) |
| S. pneumoniae | 0.1 μg | AIPO₄ | 100 | μg | 0, 3 | 5 |
| serotype 3 | | | | | | |
| S. pneumoniae | 0.1 μg | Free TLR7/8 | 2 | nmol | 0, 3 | 5 |
| serotype 3 | | | | | | |
| S. pneumoniae | 0.1 μg | Compound 1 | 2 | nmol | 0, 3 | 5 |
| serotype 3 | | | | | | |
| S. pneumoniae | 0.1 μg | Compound 2 | 2 | nmol | 0, 3 | 5 |

serotype 3

The mouse studies shown in Table 4 were completed as follows. Briefly, 15 female Swiss-Webster mice (Taconic Bioscines™) per group, aged 6-8 weeks, were immunized intramuscularly (IM) at week 0 and at 3 weeks. The mice received 50 μl of 0.1 μg of the S. pneumoniae serotype 3 antigen with one of the 4 adjuvants listed in Table 4. At 5 weeks post prime vaccination, all mice were terminally bled while under anesthesia and the sera was collected to evaluate in a S. pneumoniae serotype 3 specific opsonophagocytic activity (OPA) assay.

The OPA assays were designed to measure functional S. pneumoniae serotype 3 specific antibodies present in the collected serum. Briefly, heat-inactivated mouse sera were serially diluted 2.5-fold in Hank's balanced saline solution (Gibco™) supplemented with 0.1% gelatin and calcium/magnesium. Target bacteria were added to assay plates and incubated on a shaker for 30 min at 25° C. or 37° C. During this incubation, bacteria were coated with antibodies. Baby rabbit complement (Pel-Freez® Biologicals), at 3 to 4 weeks old and a pre-determined final concentration of 12%, 9%, or 6%, as well as differentiated HL-60 cells (ATCC®) were then added to each well and the assay plates were incubated with shaking for 45 minutes at 37° C.+/−CO2. During the second incubation, effector cells phagocytosed and killed opsonized bacteria. At the conclusion of the assay, aliquots of the assay reaction mixture were plated onto microcolony filter plates, incubated at 37° C. and 5% CO2 overnight. The resulting colonies were then stained with Coomassie® Brilliant Blue. Colonies were imaged and enumerated on a Cellular Technology Limited (CTL) ImmunoSpot® Analyzer.

The interpolated OPA antibody titer is the reciprocal of the dilution that yields a 50% reduction in the number of bacterial colonies when compared to the control wells that did not contain serum. The OPA geometric mean titers (GMT) obtained from this study are shown in Table 5 below.

TABLE 5
OPA geometric mean titers (GMT)

| Animal | | Antigen | | Adjuvant | | | Lower | Upper |
|---|---|---|---|---|---|---|---|---|
| model | Antigen | dose | Adjuvant | dose | | GMT | 95% CI | 95% CI |
| Mouse | S. pneumoniae | 0.1 μg | AlPO₄ | 100 | μg | 178 | 49 | 640 |
| | serotype 3 | | | | | | | |
| | S. pneumoniae | 0.1 μg | Free TLR7/8 | 2 | nmol | 126 | 38 | 423 |
| | serotype 3 | | | | | | | |
| | S. pneumoniae | 0.1 μg | Compound 1 | 2 | nmol | 833 | 322 | 2150 |
| | serotype 3 | | | | | | | |
| | S. pneumoniae | 0.1 μg | Compound 2 | 2 | nmol | 610 | 198 | 1881 |
| | serotype 3 | | | | | | | |

In this study, the ability of Compounds 1 and 2 to adjuvant S. pneumoniae serotype 3 was compared to aluminum phosphate and a free TLR 7/8 agonist molecule (a TLR 7/8 agonist molecule without a cholesterol or tocopherol group). The results shown in Table 5 demonstrate that both Compound 1 and Compound 2 elicited higher average OPA geometric mean titers than aluminum phosphate, as well as the free TLR 7/8 agonist molecule. Accordingly, it was concluded that Compound 1 and 2 are effective in adjuvanting the immune response to S. pneumoniae serotype 3 in a mouse model.

It will be apparent to those skilled in the art that various modifications and variations may be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All references cited herein, including patents, patent applications, papers, textbooks, and the like, and the references cited therein, to the extent that they are not already, are hereby incorporated by reference in their entireties. In the event that one or more of the incorporated literature and similar materials differs from or contradicts this application, including but not limited to defined terms, term usage, described techniques, or the like, this application controls.

1. A compound of Formula (I) 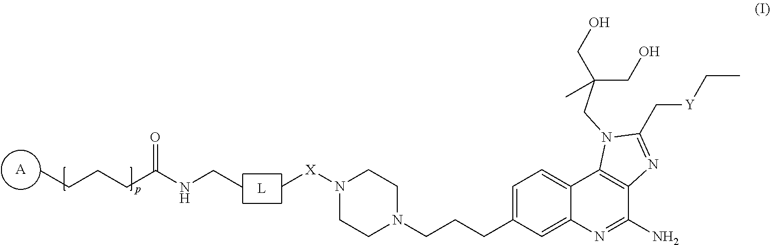
or a pharmaceutically acceptable salt, tautomer, or stereoisomer thereof, wherein
A is an albumin-binding moiety;
L is a linker;
X is C═O or CH₂;
Y is O or CH₂; and
p is an integer from 0 to 1.

2. The compound of claim 1, wherein
A is cholesterol or tocopherol, and
further wherein
if A is cholesterol, p is 0, and
if A is tocopherol, p is 1.

3. The compound of claim 2, wherein the tocopherol is selected from the group consisting of:
(i) alpha-tocopherol 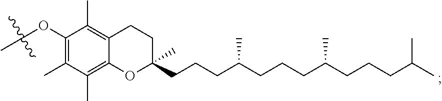 [SMILES: *Oc1c(C)c(C)c2c(c1C)CC[C@@](C)(CCC[C@H](C)CCC[C@H](C)CCCC(C)C)O2]
(ii) beta-tocopherol 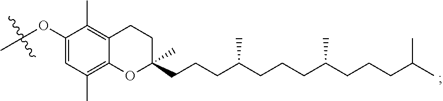 [SMILES: *Oc1cc(C)c2c(c1C)CC[C@@](C)(CCC[C@H](C)CCC[C@H](C)CCCC(C)C)O2]
(iii) gamma-tocopherol 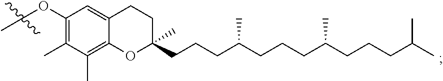 [SMILES: *Oc1cc2c(c(C)c1C)O[C@](C)(CCC[C@H](C)CCC[C@H](C)CCCC(C)C)CC2]
and
(iv) delta-tocopherol

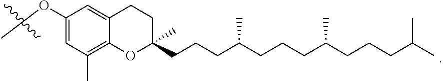 [SMILES: *Oc1cc(C)c2c(c1)CC[C@@](C)(CCC[C@H](C)CCC[C@H](C)CCCC(C)C)O2]

4. The compound of claim 1 having Formula (Ia) 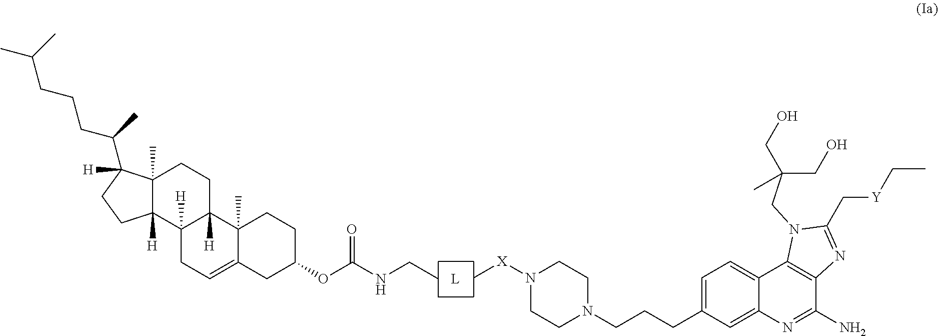
or a pharmaceutically acceptable salt, tautomer, or stereoisomer thereof.

5. The compound of claim 4, wherein L has the following structure: 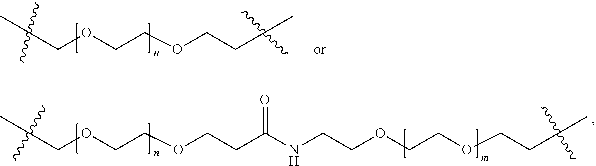
wherein
m is an integer from 1 to 35, and
n is an integer from 0 to 30.

6. The compound of claim 5 having Formula (Ia(i)) 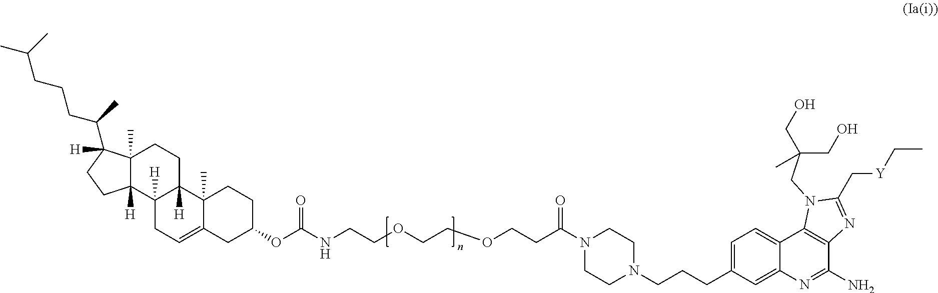
or a pharmaceutically acceptable salt, tautomer, or stereoisomer thereof.

7. The compound of claim 5 having Formula (Ia(ii)) 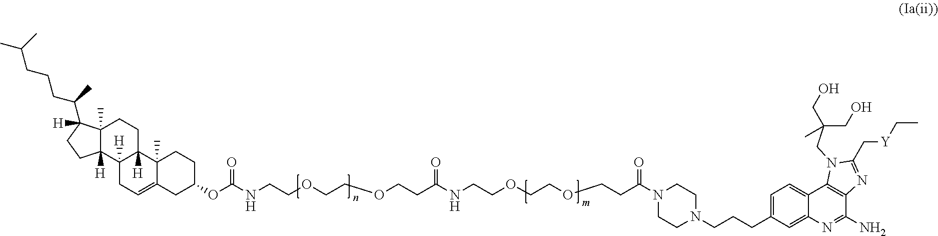
or a pharmaceutically acceptable salt, tautomer, or stereoisomer thereof.

8. The compound of claim 5 having Formula (Ia(iii)) 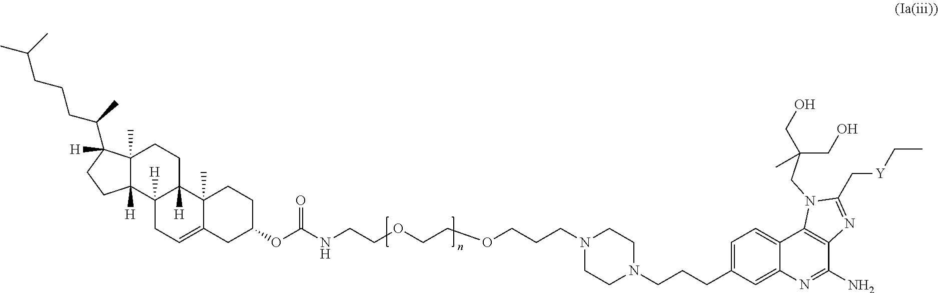
or a pharmaceutically acceptable salt, tautomer, or stereoisomer thereof.

9. The compound of claim 5 having Formula (Ia(iv)) 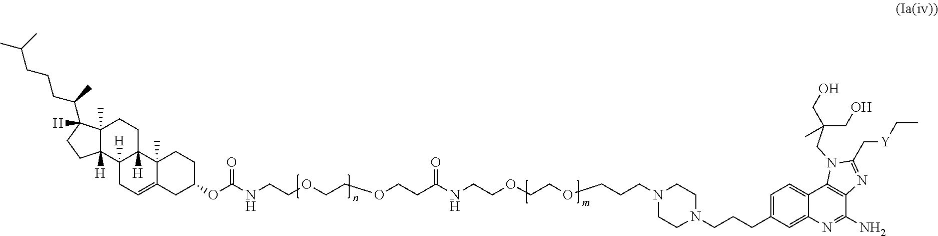
or a pharmaceutically acceptable salt, tautomer, or stereoisomer thereof.

10. The compound of claim 2 having Formula (Ib) 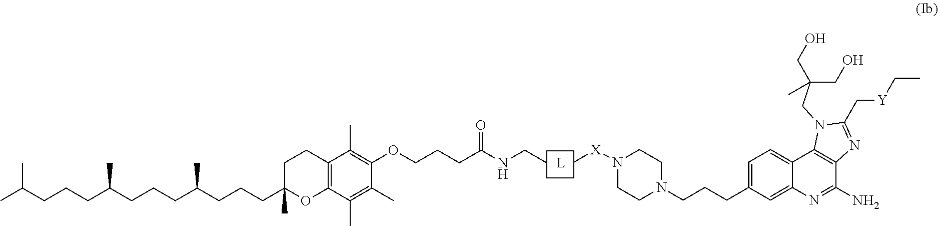
or a pharmaceutically acceptable salt, tautomer, or stereoisomer thereof.

11. The compound of claim 10, wherein L has the following structure: 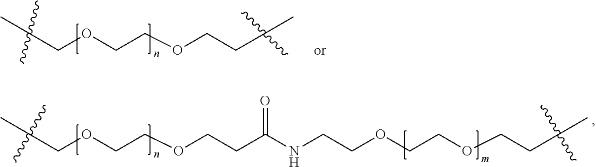
wherein
m is an integer from 1 to 35, and
n is an integer from 0 to 30.

12. The compound of claim 11:
having a Formula selected from the group consisting of Formula (Ib(i)), Formula (Ib(ii)), Formula (Ib(iii)), and Formula (Ib(iv)) 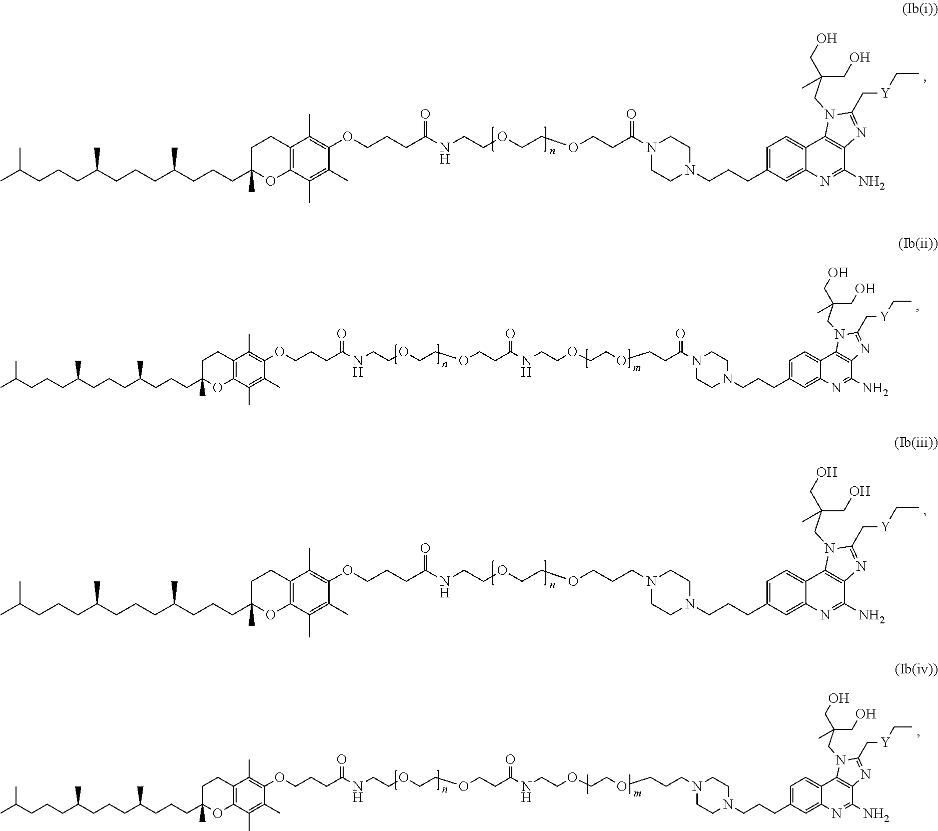
or a pharmaceutically acceptable salt, tautomer, or stereoisomer thereof.

13. (canceled)

14. (canceled)

15. (canceled)

16. (canceled)

17. The compound of claim 1, wherein L is a cleavage linker with an amide bond.

18. The compound of claim 1, wherein L has the following structure: 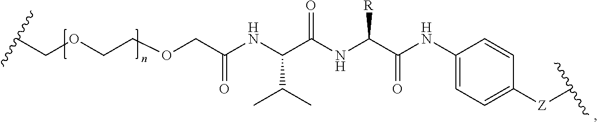
wherein
R is CH₃ or 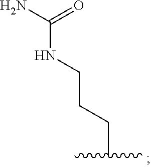 [SMILES: *CCCNC(N)=O]
and
Z is CH₂O; or
L has the following structure: 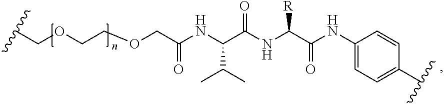
wherein
R is CH₃ or

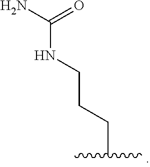 [SMILES: *CCCNC(N)=O]

19. (canceled)

20. The compound of claim 1, wherein the compound is selected from the group consisting of:
(3beta)-cholest-5-en-3-yl {91-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-15,91-dioxo-3,6,9,12,19,22,25,28,31,34,37,40,43,46,49,52,55,58,61,64,67,70,73,76,79,82,85,88-octacosaoxa-16-azahennonacont-1-yl}carbamate;
N-{75-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-75-oxo-3,6,9,12,15,18,21,24,27,30,33,36,39,42,45,48,51,54,57,60,63,66,69,72-tetracosaoxapentaheptacont-1-yl}-17-oxo-20-({(2R)-2,5,7,8-tetramethyl-2-[(4R,8R)-4,8,12-trimethyltridecyl]-3,4-dihydro-2H-chromen-6-yl}oxy)-4,7,10,13-tetraoxa-16-azaicosan-1-amide;
(3beta)-cholest-5-en-3-yl {127-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-15,127-dioxo-3,6,9,12,19,22,25,28,31,34,37,40,43,46,49,52,55,58,61,64,67,70,73,76,79,82,85,88,91,94,9 7,100,103,106,109,112,115,118,121,124-tetracontaoxa-16-azaheptacosahect-1-yl}carbamate;
(3beta)-cholest-5-en-3-yl {127-[4-(3-{4-amino-2-(ethoxymethyl)-1-[3-hydroxy-2-(hydroxymethyl)-2-methylpropyl]-1H-imidazo[4,5-c]quinolin-7-yl}propyl)piperazin-1-yl]-15,127-dioxo-3,6,9,12,19,22,25,28,31,34,37,40,43,46,49,52,55,58,61,64,67,70,73,76,79,82,85,88,91,94,9 7,100,103,106,109,112,115,118,121,124-tetracontaoxa-16-azaheptacosahect-1-yl}carbamate;
(3beta)-cholest-5-en-3-yl {91-[4-(3-{4-amino-2-(ethoxymethyl)-1-[3-hydroxy-2-(hydroxymethyl)-2-methylpropyl]-1H-imidazo[4,5-c]quinolin-7-yl}propyl)piperazin-1-yl]-15,91-dioxo-3,6,9,12,19,22,25,28,31,34,37,40,43,46,49,52,55,58,61,64,67,70,73,76,79,82,85,88-octacosaoxa-16-azahennonacont-1-yl}carbamate;
(3beta,20R)-cholest-5-en-3-yl {55-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-15,55-dioxo-3,6,9,12,19,22,25,28,31,34,37,40,43,46,49,52-hexadecaoxa-16-azapentapentacont-1-yl}carbamate;
(3beta)-cholest-5-en-3-yl {63-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-3,6,9,12,15,18,21,24,27,30,33,36,39,42,45,48,51,54,57,60-icosaoxatrihexacont-1-yl}carbamate;
(3beta,20R)-cholest-5-en-3-yl {151-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-75,151-dioxo-3,6,9,12,15,18,21,24,27,30,33,36,39,42,45,48,51,54,57,60,63,66,69,72,79,82,85,88,91,94,9 7,100,103,106,109,112,115,118,121,124,127,130,133,136,139,142,145,148-octatetracontaoxa-76-azahenpentacontahect-1-yl}carbamate;
(3beta)-cholest-5-en-3-yl {75-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-75-oxo-3,6,9,12,15,18,21,24,27,30,33,36,39,42,45,48,51,54,57,60,63,66,69,72-tetracosaoxapentaheptacont-1-yl}carbamate;
(3beta,20R)-cholest-5-en-3-yl (2-{3-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]propoxy}ethyl)carbamate;
(3beta)-cholest-5-en-3-yl {167-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-15,91,167-trioxo-3,6,9,12,19,22,25,28,31,34,37,40,43,46,49,52,55,58,61,64,67,70,73,76,79,82,85,88,95,98,1 01,104,107,110,113,116,119,122,125,128,131,134,137,140,143,146,149,152,155,158,161,1 64-dopentacontaoxa-16,92-diazaheptahexacontahect-1-yl}carbamate;
(3beta,20R)-cholest-5-en-3-yl {15-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-15-oxo-3,6,9,12-tetraoxapentadec-1-yl}carbamate;
(3beta,20R)-cholest-5-en-3-yl {55-[4-(3-{4-amino-2-(ethoxymethyl)-1-[3-hydroxy-2-(hydroxymethyl)-2-methylpropyl]-1H-imidazo[4,5-c]quinolin-7-yl}propyl)piperazin-1-yl]-15,55-dioxo-3,6,9,12,19,22,25,28,31,34,37,40,43,46,49,52-hexadecaoxa-16-azapentapentacont-1-yl}carbamate;
(3beta,20R)-cholest-5-en-3-yl {51-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-3,6,9,12,15,18,21,24,27,30,33,36,39,42,45,48-hexadecaoxahenpentacont-1-yl}carbamate;
(20R)-cholest-5-en-3beta-yl {31-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-15-oxo-3,6,9,12,19,22,25,28-octaoxa-16-azahentriacontan-1-yl}carbamate;
(20R)-cholest-5-en-3beta-yl {75-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-3,6,9,12,15,18,21,24,27,30,33,36,39,42,45,48,51,54,57,60,63,66,69,72-tetracosaoxapentaheptacontan-1-yl}carbamate;
(3beta)-cholest-5-en-3-yl {91-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-15-oxo-3,6,9,12,19,22,25,28,31,34,37,40,43,46,49,52,55,58,61,64,67,70,73,76,79,82,85,88-octacosaoxa-16-azahennonacont-1-yl}carbamate;
(20R)-cholest-5-en-3beta-yl [2-(2-{3-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]propoxy}ethoxy)ethyl]carbamate;
(20R)-cholest-5-en-3beta-yl {27-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-27-oxo-3,6,9,12,15,18,21,24-octaoxaheptacosan-1-yl}carbamate;
(20R)-cholest-5-en-3beta-yl {39-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-39-oxo-3,6,9,12,15,18,21,24,27,30,33,36-dodecaoxanonatriacontan-1-yl}carbamate;
(20R)-cholest-5-en-3beta-yl {39-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-3,6,9,12,15,18,21,24,27,30,33,36-dodecaoxanonatriacontan-1-yl}carbamate;
(20R)-cholest-5-en-3beta-yl {15-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-3,6,9,12-tetraoxapentadecan-1-yl}carbamate;
(20R)-cholest-5-en-3beta-yl {27-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-3,6,9,12,15,18,21,24-octaoxaheptacosan-1-yl}carbamate;
N-{111-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-111-oxo-3,6,9,12,15,18,21,24,27,30,33,36,39,42,45,48,51,54,57,60,63,66,69,72,75,78,81,84,87,90,9 3,96,99,102,105,108-hexatriacontaoxahendecahectan-1-yl}-17-oxo-20-({(2R)-2,5,7,8-tetramethyl-2-[(4R,8R)-4,8,12-trimethyltridecyl]-3,4-dihydro-2H-1-benzopyran-6-yl}oxy)-4,7,10,13-tetraoxa-16-azaicosan-1-amide;
N-{39-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-39-oxo-3,6,9,12,15,18,21,24,27,30,33,36-dodecaoxanonatriacontan-1-yl}-17-oxo-20-({(2R)-2,5,7,8-tetramethyl-2-[(4R,8R)-4,8,12-trimethyltridecyl]-3,4-dihydro-2H-1-benzopyran-6-yl}oxy)-4,7,10,13-tetraoxa-16-azaicosan-1-amide;
N-{75-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-3,6,9,12,15,18,21,24,27,30,33,36,39,42,45,48,51,54,57,60,63,66,69,72-tetracosaoxapentaheptacontan-1-yl}-17-oxo-20-({(2R)-2,5,7,8-tetramethyl-2-[(4R,8R)-4,8,12-trimethyltridecyl]-3,4-dihydro-2H-1-benzopyran-6-yl}oxy)-4,7,10,13-tetraoxa-16-azaicosan-1-amide;
(3beta)-cholest-5-en-3-yl {(2S,5S)-1-((4-((4-(3-(4-amino-2-butyl-1-(3-hydroxy-2-(hydroxymethyl)-2-methylpropyl)-1H-imidazo[4,5-c]quinolin-7-yl)propyl)piperazin-1-yl)methyl)phenyl)amino)-5-isopropyl-2-methyl-1,4,7-trioxo-9,12-dioxa-3,6-diazatetradecan-14-yl}carbamate;
4-((12S,15S)-1-(((3beta)-cholest-5-en-3-yl)oxy)-12-isopropyl-1,10,13-trioxo-15-(3-ureidopropyl)-5,8-dioxa-2,11,14-triazahexadecan-16-amido)benzyl 4-(3-(4-amino-2-butyl-1-(3-hydroxy-2-(hydroxymethyl)-2-methylpropyl)-1H-imidazo[4,5-c]quinolin-7-yl)propyl)piperazine-1-carboxylate; and
(3beta)-cholest-5-en-3-yl ((6S,9S)-1-amino-6-((4-((4-(3-(4-amino-2-butyl-1-(3-hydroxy-2-(hydroxymethyl)-2-methylpropyl)-1H-imidazo[4,5-c]12uinoline-7-yl)propyl)piperazin-1-yl)methyl)phenyl)carbamoyl)-9-isopropyl-1,8,11-trioxo-13,16-dioxa-2,7,10-triazaoctadecan-18-yl)carbamate;
or a pharmaceutically acceptable salt thereof.

21. A compound, which is (3beta)-cholest-5-en-3-yl {91-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-15,91-dioxo-3,6,9,12,19,22,25,28,31,34,37,40,43,46,49,52,55,58,61,64,67,70,73,76,79,82,85,88-octacosaoxa-16-azahennonacont-1-yl}carbamate, or a pharmaceutically acceptable salt thereof.

22. A compound, which is N-{75-[4-(3-{4-amino-2-butyl-1-[3-hydroxy-2-(hydroxymethyl)-2-methylpropyl]-1H-imidazo[4,5-c]quinolin-7-yl}propyl)piperazin-1-yl]-75-oxo-3,6,9,12,15,18,21,24,27,30,33,36,39,42,45,48,51,54,57,60,63,66,69,72-tetracosaoxapentaheptacont-1-yl}-17-oxo-20-({(2R)-2,5,7,8-tetramethyl-2-[(4R,8R)-4,8,12-trimethyltridecyl]-3,4-dihydro-2H-chromen-6-yl}oxy)-4,7,10,13-tetraoxa-16-azaicosan-1-amide, or a pharmaceutically acceptable salt thereof.

23. A pharmaceutical composition comprising the compound according to claim 1, or a pharmaceutically acceptable salt thereof, and at least one pharmaceutically acceptable excipient.

24. A method of inducing an immune response to an antigen of interest in a subject, comprising administering to the subject the pharmaceutical composition of claim 23, wherein the composition further comprises the antigen of interest; or
a method for immunizing a subject against a disease or disorder caused by or associated with an antigen of interest, comprising administering to the subject the pharmaceutical composition of claim 23, wherein the composition further comprises the antigen of interest; or
a method for preventing a disease or disorder caused by or associated with an antigen of interest in a subject, comprising administering to the subject the pharmaceutical composition of claim 23, wherein the composition further comprises the antigen of interest; or
a method for treating a disease or disorder caused by or associated with an antigen of interest in a subject, comprising administering to the subject the pharmaceutical composition of claim 23, wherein the composition further comprises the antigen of interest; or
a method for increasing an immune response to an antigen of interest in a subject, comprising administering to the subject the pharmaceutical composition of claim 23, wherein the composition further comprises the antigen of interest.

25-58. (canceled)

59. A crystal comprising the compound according to claim 1 or a pharmaceutically acceptable salt thereof.


SEQUENCE LISTING (4 sequences)

SEQ ID NO: 1 - protein, 84 aa, synthetic construct
  source 1..84: note = synthetic polypeptide
QNITEEFYQSTCSAVSKGYLSALRTGWYTSVITIELSNIKENKCNGTDAKVKLIKQELDKYKNAVTELQLLMQSTPACNSRARR

SEQ ID NO: 2 - protein, 377 aa, synthetic construct
  source 1..377: note = synthetic polypeptide
FLGFLLGVGSACASGIAVSKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSSNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSLAFIRKSDELL

SEQ ID NO: 3 - protein, 84 aa, synthetic construct
  source 1..84: note = synthetic polypeptide
QNITEEFYQSTCSAVSRGYFSALRTGWYTSVITIELSNIKETKCNGTDTKVKLIKQELDKYKNAVTELQLLMQNTPACNNRARR

SEQ ID NO: 4 - protein, 377 aa, synthetic construct
  source 1..377: note = synthetic polypeptide
FLGFLLGVGSACASGIAVSKVLHLEGEVNKIKNALLSTNKAVVSLSNGVSVLTIKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSSEFDASISQVNEKINQSLAFIRRSDELL